(19) United States
(12) Patent Application Publication
(10) Pub. No.: US 2019/0175690 A1
(43) Pub. Date: Jun. 13, 2019

(54) COMPOSITIONS AND METHODS FOR CARDIAC REPAIR

(71) Applicant: CHILDREN'S MEDICAL CENTER CORPORATION, BOSTON, MA (US)

(72) Inventors: WILLIAM PU, BOSTON, MA (US); ZHIQIANG LIN, BOSTON, MA (US)

(73) Assignee: CHILDREN'S MEDICAL CENTER CORPORATION, BOSTON, MA (US)

(21) Appl. No.: 16/326,108

(22) PCT Filed: Aug. 16, 2017
(86) PCT No.: PCT/US17/47200

§ 371 Date: Feb. 15, 2019

Related U.S. Application Data
(60) Provisional application No. 62/375,799, filed on Aug. 16, 2016.

(51) Int. Cl. A61K 38/17; A61K 31/336; A61K 9/00; A61P 9/00

(52) U.S. Cl. CPC A61K 38/1709; A61P 9/00; A61K 9/0019; A61K 31/336

(57) ABSTRACT

The present invention features compositions comprising a modified RNA encoding a Yes-associated protein (YAP) polypeptide and methods of using such compositions for cardiac repair. In particular embodiments, a modified RNA encoding a YAP polypeptide is administered in combination with an agent that reduces YAP degradation, such as a small molecule E64d.

### CROSS-REFERENCE TO RELATED APPLICATIONS

This application claims the benefit of and priority to U.S. Provisional Patent Application Ser. No. 62/375,799 filed Aug. 16, 2016 which is incorporated herein by reference in its entirety.

### STATEMENT OF RIGHTS TO INVENTIONS MADE UNDER FEDERALLY SPONSORED RESEARCH

This invention was made with government support under Grant Nos: NIH HL116461, HL100401, and U01HL198166 awarded by the National Institutes of Health. The government has certain rights in the invention.

### BACKGROUND OF THE INVENTION

Proper control of organ growth is fundamental to animal development and organ homeostasis. Unrestrained activity of growth promoting pathways leads to cancer, whereas targeted activation of these pathways may be a strategy for organ regeneration. One area with great need for advances in regenerative medicine is heart disease. Heart failure is the world's leading cause of death, and the prevalence of heart failure is expected to further increase as the population ages. Cardiomyocyte (CM) loss is a central pathogenic mechanism in heart failure, but limited endogenous regenerative capacity in the adult heart has precluded development of therapeutic approaches to efficiently replace these lost CMs. Unlike adult CMs, fetal CMs robustly proliferate to match the rapid growth of the embryo. Methods for inducing growth and/or regeneration in adult cardiomyocytes would provide therapies for patients that would otherwise suffer and die from heart failure and related cardiac disorders.

### SUMMARY OF THE INVENTION

As described below, the present invention features compositions comprising a modified RNA encoding a Yes-associated protein (YAP) polypeptide and methods of using the compositions for transient expression of a YAP polypeptide to promote cardiac repair in a subject in need thereof. In particular embodiments, a modified RNA encoding a YAP polypeptide is administered in combination with an agent that reduces YAP degradation, such as the small molecule E64d.

In one aspect, the invention provides a method of inducing regeneration and/or reducing cardiomyocyte loss or cell death in a cardiac tissue of a subject. The method involves transiently increasing the level, expression, or activity of Yap in a cell or progenitor thereof in the subject, thereby inducing regeneration and/or reducing cell death (e.g., by apoptosis or necrosis) in the cardiac tissue.

A method of increasing cardiac function or reducing cardiac hypertrophy in a subject following ischemic reperfusion injury, the method involving administering to the subject a YAP polypeptide or polynucleotide encoding the polypeptide, thereby increasing cardiac function or reducing cardiac hypertrophy in the subject. In another aspect, the invention provides a method for expressing a YAP protein in a cell involving contacting the cell with a synthetic, modified RNA molecule encoding a YAP polypeptide or a composition containing a synthetic, modified RNA molecule encoding a YAP polypeptide.

In another aspect, the invention provides a method of treating myocardial infarction or a symptom thereof involving transiently administering to a subject an agent that inhibits cathepsin B (e.g., E64d) after myocardial infarction, thereby treating myocardial infarction or a symptom thereof.

In various embodiments of any aspect delineated herein, a modified RNA molecule encoding a YAP polypeptide is used to transiently increase the level, expression, or activity of YAP in a cell or progenitor thereof. In various embodiments of any aspect delineated herein, the synthetic, modified RNA molecule encoding a YAP polypeptide comprises at least two modified nucleosides. In various embodiments, the at least two modified nucleosides include one or more of 5-methylcytidine (5mC), N6-methyladenosine (m6A), 3,2′-O-dimethyluridine (m4U), 2-thiouridine (s2U), 2′ fluorouridine, pseudouridine, N1-methyl-pseudouridine, 2′-O-methyluridine (Um), 2′ deoxy uridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m-2,7G), N2,N2,7-trimethylguanosine (m-2,2,7G), and inosine (I).

In various embodiments of any aspect delineated herein, the modified RNA molecule encoding a YAP polypeptide further has a 5′ cap or a 5′ cap analog and/or does not have a 5′ triphosphate. In various embodiments of any aspect delineated herein, the synthetic, modified RNA molecule further has a poly(A) tail, a Kozak sequence, a 3′ untranslated region, a 5′ untranslated region, or any combination thereof, where the poly(A) tail, Kozak sequence, 3′ untranslated region, 5′ untranslated region can optionally have one or modified nucleosides selected from 5-methylcytidine (5mC), N6-methyladenosine (m6A), 3,2′-O-dimethyluridine (m4U), 2-thiouridine (s2U), 2′ fluorouridine, pseudouridine, N1-methyl-pseudouridine, 2′-O-methyluridine (Um), 2′ deoxy uridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m-2,7G), N2,N2,7-trimethylguanosine (m-2,2,7G), and inosine (I). In various embodiments of any aspect delineated herein, the method further involves administering E64d to the subject. In various embodiments of any aspect delineated herein, E64d is used to transiently increase the level, expression, or activity of Yap in a cell or progenitor thereof.

In various embodiments of any aspect delineated herein, the cell is in vitro or in vivo. In various embodiments of any aspect delineated herein, the cell is present in a tissue. In various embodiments of any aspect delineated herein, the cell is derived from heart tissue, cardiac tissue, or muscle tissue.

In various embodiments of any aspect delineated herein, the synthetic, modified RNA molecule encoding a YAP polypeptide is not expressed in a vector or is naked synthetic, modified RNA molecule.

In various embodiments of any aspect delineated herein, the composition comprising the synthetic, modified RNA molecule encoding a YAP polypeptide is present in a lipid complex. In various embodiments of any aspect delineated herein, the composition contains a concentration of synthetic, modified RNA molecule of greater than 100 ng/μl. In various embodiments of any aspect delineated herein, the composition contains a concentration of synthetic, modified RNA molecule of between 1-25 μg/μl.

In various embodiments of any aspect delineated herein, the modified RNA or composition is administered to the tissue by direct injection, contacting the tissue with an implantable device containing, or coated with the synthetic, modified RNA molecule, and/or delivering the synthetic, modified RNA molecule via a catheter or an endoscope. In some embodiments, the catheter is a Balloon Catheter. In various embodiments, the implantable device is a stent or implantable delivery pump.

In various embodiments of any aspect delineated herein, the subject has or is at risk for developing a myocardial infarction, congestive heart failure, cardiomyopathy, myocardial infarction, tissue ischemia, cardiac ischemia, tissue repair, and trauma injury. In various embodiments of any aspect delineated herein, the subject has had or is planning to have cardiac surgery, has an ischemia condition, or is in need of a stent placement.

In various embodiments of any aspect above or delineated herein, the agent is a epoxysuccinyl, vinyl sulfone or nitrile based compound (e.g., E64d, E64c, JPM-OEt, CA-030, CA-074, NS134, NS-629, LNC-NS-629, PK1, and ASM7).

In various embodiments of any aspect delineated herein, the method further involves administering to the subject an agent that inhibits cathepsin B. In various embodiments of any aspect delineated herein, the YAP polypeptide comprises one or more activating mutations (e.g., S61A, S109A, S127A, S164A, S381A, S127A mutation, W1, W2, and W1W2).

In various embodiments of any aspect delineated herein, the level, expression, or activity of YAP in a cell is increased for about 3 mos., 2 mos., 1 mo., 4 weeks, 3 weeks, 2 weeks, 7 days, 6 days, 5 days, 4 days, 3 days, 2 days, 1 day, 24 hr., 23 hr., 22 hr., 21 hr., 20 hr., 19 hr., 18 hr., 17 hr., 16 hr., 15 hr., 14 hr., 13 hr., 12 hr., 11 hr., 10 hr., 9 hr., 8 hr., 7 hr., 6 hr., 5 hr., 4 hr., 3 hr., 2 hr., 1 hr., 60 min., 45 min., 30 min., 15 min., 10 min., 9 min., 8 min., 7 min., 6 min., 5 min., 4 min., 3 min., 2 min., 1 min., or less after injury or damage to the heart (e.g., myocardial infarction, congestive heart failure, cardiomyopathy, myocardial infarction, tissue ischemia, cardiac ischemia, planned cardiac surgery, stent placement). In various embodiments, the increase in the level, expression, or activity of YAP in a cell is not constitutive.

In various embodiments, the agent is administered within about 3 mos., 2 mos., 1 mo., 4 weeks, 3 weeks, 2 weeks, 7 days, 6 days, 5 days, 4 days, 3 days, 2 days, 1 day, 24 hr., 23 hr., 22 hr., 21 hr., 20 hr., 19 hr., 18 hr., 17 hr., 16 hr., 15 hr., 14 hr., 13 hr., 12 hr., 11 hr., 10 hr., 9 hr., 8 hr., 7 hr., 6 hr., 5 hr., 4 hr., 3 hr., 2 hr., 1 hr., 60 min., 45 min., 30 min., 15 min., 10 min., 9 min., 8 min., 7 min., 6 min., 5 min., 4 min., 3 min., 2 min., 1 min., immediately after, or at the time of injury or damage to the heart (e.g., myocardial infarction, congestive heart failure, cardiomyopathy, myocardial infarction, tissue ischemia, cardiac ischemia).

In various embodiments, the agent is administered about 3 mos., 2 mos., 1 mo., 4 weeks, 3 weeks, 2 weeks, 7 days, 6 days, 5 days, 4 days, 3 days, 2 days, 1 day, 24 hr., 23 hr., 22 hr., 21 hr., 20 hr., 19 hr., 18 hr., 17 hr., 16 hr., 15 hr., 14 hr., 13 hr., 12 hr., 11 hr., 10 hr., 9 hr., 8 hr., 7 hr., 6 hr., 5 hr., 4 hr., 3 hr., 2 hr., 1 hr., 60 min., 45 min., 30 min., 15 min., 10 min., 9 min., 8 min., 7 min., 6 min., 5 min., 4 min., 3 min., 2 min., 1 min., immediately prior to or at the time of cardiac surgery (e.g., planned cardiac surgery, stent placement, etc.).

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “YAP polypeptide is meant a protein having about 85% or greater amino acid sequence identity to NCBI Accession No. P46937, or a fragment thereof, and having chromatin binding or transcriptional regulatory activity.

The sequence of an exemplary YAP protein is provided below:

>sp|P46937|YAP1_HUMAN Transcriptional coactivator
YAP1 OS = Homo sapiens GN = YAP1 PE = 1 SV = 2
MDPGQQPPPQPAPQGQGQPPSQPPQGQGPPSGPGQPAPAATQAAPQAPPA
GHQIVHVRGDSETDLEALFNAVMNPKTANVPQTVPMRLRKLPDSFFKPPE
PKSHSRQASTDAGTAGALTPQHVRAHSSPASLQLGAVSPGTLTPTGVVSG
PAATPTAQHLRQSSFEIPDDVPLPAGWEMAKTSSGQRYFLNHIDQTTTWQ
DPRKAMLSQMNVTAPTSPPVQQNMMNSASGPLPDGWEQAMTQDGETYYIN
HKNKTTSWLDPRLDPRFAMNQRISQSAPVKQPPPLAPQSPQGGVMGGSNS
NQQQQMRLQQLQMEKERLRLKQQELLRQAMRNINPSTANSPKCQELALRS
QLPTLEQDGGTQNPVSSPGMSQELRTMTTNSSDPFLNSGTYHSRDESTDS
GLSMSSYSVPRTPDDFLNSVDEMDTGDTINQSTLPSQQNRFPDYLEAIPG
TNVDLGTLEGDGMNIEGEELMPSLQEALSSDILNDMESVLAATKLDKESF
LTWL

In one embodiment, the YAP polypeptide comprises a mutation. In another embodiment, the mutation is an activating S127A mutation (aYAP). In other embodiments, the activating mutation is S61A, S109A, S127A, S164A, S381A, S127A mutation, W1, W2, and W1W2, or combinations thereof. Such mutations are known in the art and described, for example, by Zhao et al., Cancer Res. 2009, 69:1089-1098; and Zhao et al., Genes Dev. 2011 25:51-63, each of which is incorporated herein in its entirety. Activating YAP mutations increase protein activity, for example, by enhancing nuclear localization or reducing protein degradation.

By “YAP polynucleotide” is meant a nucleic acid molecule encoding a YAP polypeptide or fragment thereof. The sequence of an exemplary YAP polynucleotide is provided below.
LOCUS NM_006106 5234 bp mRNA linear PRI 22-NOV-2015 DEFINITION Homo sapiens Yes associated protein 1 (YAP1), transcript variant 2 mRNA.gene=“YAP1” gene_synonym=“COB1; YAP; YAP2; YAP65; YKI” ORIGIN

| | |
|---|---|
| 1 | gccgccgcca gggaaaagaa agggaggaag gaaggaacaa gaaaaggaaa taaagagaaa |
| 61 | ggggaggcgg ggaaaggcaa cgagctgtcc ggcctccgtc aagggagttg gagggaaaaa |
| 121 | gttctcaggc gccgcaggtc cgagtgcctc gcagcccctc ccgaggcgca gccgccagac |
| 181 | cagtggagcc ggggcgcagg gcgggggcgg aggcgccggg gcgggggatg cggggccgcg |
| 241 | gcgcagcccc ccggccctga gagcgaggac agcgccgccc ggcccgcagc cgtcgccgct |
| 301 | tctccacctc ggcccgtgga gccggggcgt ccgggcgtag ccctcgctcg cctgggtcag |
| 361 | ggggtgcgcg tcgggggagg cagaagccat ggatcccggg cagcagccgc cgcctcaacc |
| 421 | ggccccccag ggccaagggc agccgccttc gcagcccccg caggggcagg gcccgccgtc |
| 481 | cggacccggg caaccggcac ccgcggcgac ccaggcggcg ccgcaggcac cccccgccgg |
| 541 | gcatcagatc gtgcacgtcc gcggggactc ggagaccgac ctggaggcgc tcttcaacgc |
| 601 | cgtcatgaac cccaagacgg ccaacgtgcc ccagaccgtg cccatgaggc tccggaagct |
| 661 | gcccgactcc ttcttcaagc cgccggagcc caaatcccac tcccgacagg ccagtactga |
| 721 | tgcaggcact gcaggagccc tgactccaca gcatgttcga gctcattcct ctccagcttc |
| 781 | tctgcagttg ggagctgttt ctcctgggac actgaccccc actggagtag tctctggccc |
| 841 | agcagctaca cccacagctc agcatcttcg acagtcttct tttgagatac ctgatgatgt |
| 901 | acctctgcca gcaggttggg agatggcaaa gacatcttct ggtcagagat acttcttaaa |
| 961 | tcacatcgat cagacaacaa catggcagga ccccaggaag gccatgctgt cccagatgaa |
| 1021 | cgtcacagcc cccaccagtc caccagtgca gcagaatatg atgaactcgg cttcagccat |
| 1081 | gaaccagaga atcagtcaga gtgctccagt gaaacagcca ccacccctgg ctccccagag |
| 1141 | cccacaggga ggcgtcatgg gtggcagcaa ctccaaccag cagcaacaga tgcgactgca |
| 1201 | gcaactgcag atggagaagg agaggctgcg gctgaaacag caagaactgc ttcggcagga |
| 1261 | gttagccctg cgtagccagt taccaacact ggagcaggat ggtgggactc aaaatccagt |
| 1321 | gtcttctccc gggatgtctc aggaattgag aacaatgacg accaatagct cagatccttt |
| 1381 | ccttaacagt ggcacctatc actctcgaga tgagagtaca gacagtggac taagcatgag |
| 1441 | cagctacagt gtccctcgaa ccccagatga cttcctgaac agtgtggatg agatggatac |
| 1501 | aggtgatact atcaaccaaa gcaccctgcc ctcacagcag aaccgtttcc cagactacct |
| 1561 | tgaagccatt cctgggacaa atgtggacct tggaacactg gaaggagatg gaatgaacat |
| 1621 | agaaggagag gagctgatgc caagtctgca ggaagctttg agttctgaca tccttaatga |
| 1681 | catggagtct gttttggctg ccaccaagct agataaagaa agctttctta catggttata |
| 1741 | gagccctcag gcagactgaa ttctaaatct gtgaaggatc taaggagaca catgcaccgg |
| 1801 | aaatttccat aagccagttg cagttttcag gctaatacag aaaaagatga acaaacgtcc |
| 1861 | agcaagatac tttaatcctc tattttgctc ttccttgtcc attgctgctg ttaatgtatt |
| 1921 | gctgacctct ttcacagttg gctctaaaga atcaaaagaa aaaaactttt tatttctttt |
| 1981 | gctattaaaa ctactgttca ttttgggggc tgggggaagt gagcctgttt ggatgatgga |
| 2041 | tgccattcct tttgcccagt taaatgttca ccaatcattt taactaaata ctcagactta |
| 2101 | gaagtcagat gcttcatgtc acagcattta gtttgttcaa cagttgtttc ttcagcttcc |
| 2161 | tttgtccagt ggaaaaacat gatttactgg tctgacaagc caaaaatgtt atatctgata |
| 2221 | ttaaatactt aatgctgatt tgaagagata gctgaaacca aggctgaaga ctgttttact |
| 2281 | ttcagtattt tcttttcctc ctagtgctat cattagtcac ataatgacct tgattttatt |
| 2341 | ttaggagctt ataaggcatg agacaatttc catataaata tattaattat tgccacatac |
| 2401 | tctaatatag attttggtgg ataattttgt gggtgtgcat tttgttctgt tttgttgggt |
| 2461 | tttttgtttt ttttgttttt ggcagggtcg gtgggggggt tggttggttg gttggttttg |
| 2521 | tcggaaccta ggcaaatgac catattagtg aatctgttaa tagttgtagc ttgggatggt |
| 2581 | tattgtagtt gttttggtaa aatcttcatt tcctggtttt ttttaccacc ttatttaaat |
| 2641 | ctcgattatc tgctctctct tttatataca tacacacacc caaacataac atttataata |
| 2701 | gtgtggtagt ggaatgtatc cttttttagg tttccctgct ttccagttaa tttttaaaat |
| 2761 | ggtagcgctt tgtatgcatt tagaatacat gactagtagt ttatatttca ctggtagttt |
| 2821 | aaatctggtt ggggcagtct gcagatgttt gaagtagttt agtgttctag aaagagctat |
| 2881 | tactgtggat agtgcctagg ggagtgctcc acgccctctg ggcatacggt agatattatc |
| 2941 | tgatgaattg gaaaggagca aaccagaaat ggctttattt tctcccttgg actaattttt |
| 3001 | aagtctcgat tggaattcag tgagtaggtt cataatgtgc atgacagaaa taagctttat |
| 3061 | agtggtttac cttcatttag ctttggaagt tttctttgcc ttagttttgg aagtaaattc |
| 3121 | tagtttgtag ttctcatttg taatgaacac attaacgact agattaaaat attgccttca |
| 3181 | agattgttct tacttacaag acttgctcct acttctatgc tgaaaattga ccctggatag |
| 3241 | aatactataa ggttttgagt tagctggaaa agtgatcaga ttaataaatg tatattggta |
| 3301 | gttgaattta gcaaagaaat agagataatc atgattatac ctttattttt acaggaagag |
| 3361 | atgatgtaac tagagtatgt gtctacagga gtaataatgg tttccaaaga gtatttttta |
| 3421 | aaggaacaaa acgagcatga attaactctt caatataagc tatgaagtaa tagttggttg |
| 3481 | tgaattaaag tggcaccagc tagcacctct gtgttttaag ggtctttcaa tgtttctaga |
| 3541 | ataagccctt attttcaagg gttcataaca ggcataaaat ctcttctcct ggcaaaagct |
| 3601 | gctatgaaaa gcctcagctt gggaagatag atttttttcc ccccaattac aaaatctaag |
| 3661 | tattttggcc cttcaatttg gaggagggca aaagttggaa gtaagaagtt ttattttaag |
| 3721 | tactttcagt gctcaaaaaa atgcaatcac tgtgttgtat ataatagttc ataggttgat |
| 3781 | cactcataat aattgactct aaggctttta ttaagaaaac agcagaaaga ttaaatcttg |
| 3841 | aattaagtct ggggggaaat ggccactgca gatggagttt tagagtagta atgaaattct |
| 3901 | acctagaatg caaaattggg tatatgaatt acatagcatg ttgttgggat tttttttaat |
| 3961 | gtgcagaaga tcaaagctac ttggaaggag tgcctataat ttgccagtag ccacagatta |
| 4021 | agattatatc ttatatatca gcagattagc tttagcttag ggggagggtg ggaaagtttg |
| 4081 | gggggggggt tgtgaagatt tagggggacc ttgatagaga actttataaa cttctttctc |
| 4141 | tttaataaag acttgtctta caccgtgctg ccattaaagg cagctgttct agagtttcag |
| 4201 | tcacctaagt acacccacaa aacaatatga atatggagat cttcctttac ccctcaactt |
| 4261 | taatttgccc agttatacct cagtgttgta gcagtactgt gatacctggc acagtgcttt |
| 4321 | gatcttacga tgccctctgt actgacctga aggagaccta agagtccttt ccctttttga |
| 4381 | gtttgaatca tagccttgat gtggtctctt gttttatgtc cttgttccta atgtaaaagt |
| 4441 | gcttaactgc ttcttggttg tattgggtag cattgggata agattttaac tgggtattct |
| 4501 | tgaattgctt ttacaataaa ccaattttat aatctttaaa tttatcaact ttttacattt |
| 4561 | gtgttatttt cagtcagggc ttcttagatc tacttatggt tgatggagca cattgatttg |
| 4621 | gagtttcaga tcttccaaag cactatttgt tgtaataact tttctaaatg tagtgccttt |
| 4681 | aaaggaaaaa tgaacacagg gaagtgactt tgctacaaat aatgttgctg tgttaagtat |
| 4741 | tcatattaaa tacatgcctt ctatatggaa catggcagaa agactgaaaa ataacagtaa |
| 4801 | ttaattgtgt aattcagaat tcataccaat cagtgttgaa actcaaacat tgcaaaagtg |
| 4861 | ggtggcaata ttcagtgctt aacacttttc tagcgttggt acatctgaga aatgagtgct |
| 4921 | caggtggatt ttatcctcgc aagcatgttg ttataagaat tgtgggtgtg cctatcataa |
| 4981 | caattgtttt ctgtatcttg aaaaagtatt ctccacattt taaatgtttt atattagaga |
| 5041 | attctttaat gcacacttgt caaatatata tatatagtac caatgttacc tttttatttt |
| 5101 | ttgttttaga tgtaagagca tgctcatatg ttaggtactt acataaattg ttacattatt |
| 5161 | ttttcttatg taataccttt ttgtttgttt atgtggttca aatatattct ttccttaaac |
| 5221 | tcttaaaaaa aaaa |

By “agent” is meant a peptide, polypeptide, nucleic acid molecule, or small compound. An agent that inhibits cathepsin B is an agent that reduces cathepsin B activity (e.g., by at least about 10, 25, 50, 75, or 100%. Such agents include, for example, a epoxysuccinyl, vinyl sulfone or nitrile based compound (e.g., E64d, E64c, JPM-OEt, CA-030, CA-074, NS134, NS-629, LNC-NS-629, PK1, ASM7). Agents that inhibit cathepsin B are known in the art and described, for example by Ruan H. et al, Horiz Cancer Res. 2015 ; 56: 23-40, which is incorporated herein in its entirety. By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polynucleotide analog retains the biological activity of a corresponding naturally-occurring polypeptide, while having certain biochemical modifications that enhance the analog's function relative to a naturally occurring polynucleotide. Such biochemical modifications could increase the analog's nuclease resistance, membrane permeability, or half-life, without altering, for example, functional activity, such as it's protein encoding function. An analog may include a modified nucleic acid molecule.

The term “cardiomyocyte” as used herein broadly refers to a muscle cell of the heart. The term cardiomyocyte includes smooth muscle cells of the heart, as well as cardiac muscle cells, which include also include striated muscle cells, as well as spontaneous beating muscle cells of the heart.

As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “effective amount” as used herein refers to the amount of therapeutic agent of pharmaceutical composition, e.g., an amount of the synthetic modified RNA to express sufficient amount of the protein to reduce at least one or more symptom(s) of the disease or disorder, and relates to a sufficient amount of pharmacological composition to provide the desired effect. The phrase “therapeutically effective amount” as used herein, e.g., of a synthetic modified RNA as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to, for example, effect a therapeutically or prophylatically significant reduction in a symptom or clinical marker associated with a cardiac dysfunction or disorder when administered to a typical subject who has a cardiovascular condition, disease or disorder.

With reference to the treatment of, for example, a cardiovascular condition or disease in a subject, the term “therapeutically effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., animal models of ischemia-reperfusion injury (Headrick J P, Am J Physiol Heart circ Physiol 285;H1797; 2003) and animal models acute myocardial infarction. (Yang Z, Am J Physiol Heart Circ. Physiol 282:H949: 2002; Guo Y, J Mol Cell Cardiol 33;825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals.

Subjects amenable to treatment by the methods as disclosed herein can be identified by any method to diagnose myocardial infarction (commonly referred to as a heart attack) or a cancer. Methods of diagnosing these conditions are well known by persons of ordinary skill in the art. By way of non-limiting example, myocardial infarction can be diagnosed by (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein the term “E64d” is an epoxide having the molecular formula C₁₇H₃₀N₂O₅ (CAS number 88321-09-9) and protease inhibiting activity, including cysteine proteases. The structure of E64d is provided below.

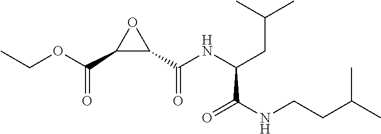 [SMILES: CCOC(=O)[C@H]1O[C@@H]1C(=O)N[C@@H](CC(C)C)C(=O)NCCC(C)C]

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

By “reducing cell death” is meant reducing the propensity or probability that a cell will die. Cell death can be apoptotic, necrotic, or by any other means.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “regeneration” means regrowth of a cell population, organ or tissue after disease or trauma.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “tissue” refers to a group or layer of similarly specialized cells which together perform certain special functions. The term “tissue-specific” refers to a source or defining characteristic of cells from a specific tissue.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1F depict developmental changes in VGLL4-TEAD1 and YAP-TEAD1 interaction in the mouse heart. FIG. 1A depicts an immunoblot (IB) of protein extracts from adult mouse brain (B), heart (H), kidney (K), liver (Li), and lung (Lu). FIG. 1B depicts an immunoblot (IB) of heart protein extracts from mice with the indicated postnatal (P) age in days. FIG. 1C is an immunoblot depicting VGLL4, TEAD1, and YAP expression in CMs and non-CMs. Adult hearts were dissociated by collagenase perfusion and then separated into CM and non-CM fractions. Protein extracts were immunoblotted with the indicated antibodies. FIG. 1D is an immunoblot depicting age-dependent association of VGLL4 and TEAD1 in mouse heart. Tead1^{fb/+};R26^{BirA/+} heart extract was incubated with immobilized streptavidin (SA). Co-precipitated VGLL4 and TEAD1 were measured by immunoblotting. Tead1^{+/+}; R26^{BirA/+} heart extract was used as a negative control. FIG. 1E is an immunoblot depicting age-dependent association of YAP and TEAD1 in mouse heart. TEAD1 was precipitated from protein from P1, P8, or P50 mouse heart as in FIG. 1D. Co-precipitated proteins were detected by immunoblotting. FIG. 1F is a graph depicting relative YAP or VGLL4 co-immunoprecipitation with TEAD1, determined by quantification of FIG. 1E. Precipitated proteins were normalized to TEAD1^{fb}.

FIGS. 2A-2F depict the construction and validation of Tead1^{fb} allele. FIG. 2A depicts a gene targeting strategy for generation of Tead1 flagbio knock-in mice (Tead1^{fb/+}). Flag and bio epitope tags were placed on the Tead1 C-terminus. Tead1 flagbio-Neo mouse was mated to ActB::Flpe mouse to remove the frt-neo-frt selection marker. FIG. 2B is a Southern Blot depicting homologous recombination in embryonic stem (ES) cells. Arrow indicates the wild type allele, and arrowhead indicates the targeted allele. Two independent Tead1 flagbio-Neo ES clones (#48 and#49) were tested. NC negative control, no genomic DNA included. WT, wild type ES genomic DNA. FIG. 2C is a chart depicting allele frequency after the removal of the Frt-neo-Frt cassette by ActB::Flpe, and intercrossing of Tead1^{fb/+} mice. Tead1^{fb/fb} mice survived normally. FIG. 2D depicts echocardiography measurement of Tead1^{fb/fb} mice heart function. 4 months old wild type mice and Tead1^{fb/fb} mice were used for heart function test. NS, no significant difference. n=4. FIG. 2E depicts western blot with adult heart tissue from indicated mice. FIG. 2F depicts a western blot with E14.5 heart tissue from indicated mice. Streptavidin HRP was used to detect biotinylated Tead1^{fb}, demonstrating in vivo biotinylation

FIGS. 3A-3F show that VGLL4 overexpression did not suppress neonatal cardiac growth. P1 pups were injected subcutaneously with AAV9.GFP or AAV9.VGLL4-GFP. Control (Ctrl) mice were untreated. Hearts were analyzed at P8. FIG. 3A depicts AAV9 expression constructs. AAV9.GFP and AAV9.VGLL4-GFP with the cardiac troponin T (cTNT) promoter incorporated to drive selective CM expression. Heart protein immunoblots (lower panel) probed with GFP antibody demonstrated VGLL4-GFP fusion protein expression (arrowhead). FIG. 3B depicts a graph of echocardiographic assessment of neonatal heart function. Percent fractional shortening (FS%). n=4. FIG. 3C depicts whole mount images of hearts showing lack of substantial differences between groups. Bar=2 mm. FIG. 3D depicts a graph of heart to body weight ratio, which was not significantly different (NS) between groups. FIG. 3E depicts representative pH3 staining results. Bar=50 μm. FIG. 3F is a graph providing quantitation of pH3+ CMs. n=3. FIG. 3G is a graph providing a quantitation of cell cycle gene expression from P8 ventricular myocardium after treatment with AAV9.GFP or AAV9.VGLL4-GFP. Gene expression was measured by qRT-PCR and normalized to the AAV9.GFP group. n=4. NS, not significant.

FIGS. 4A-4B show that TEAD1 interacts with VGLL4-GFP in adult but not neonatal heart. In FIGS. 4A-4B AAV-GFP (GFP) or AAV-VGLL4-GFP (Vgll4) were administered to 1 day old Tead1^{fb/+}, R26^{BirA/+} pups. Mouse hearts were collected at either 1 month (FIG. 4A) or P8 (FIG. 4B) after AAV administration (B). Tead1^{fb} was pulled down on SA beads, and co-precipitated VGLL4-GFP was analyzed by western blotting. GAPDH or Ponceau S were used as loading controls.

FIGS. 5A-5L show VGLL4 TDU domain acetylation decreased VGLL4-TEAD1 interaction. FIG. 5A depicts the results of a co-precipitation experiment where p300 bound and acetylated VGLL4. HEK293T cells were transfected with the indicated GFP and histone acetyltransferase (HAT; HA-tagged) expression plasmids. Proteins that co-precipitated with GFP were detected by immunoblotting (IB). K—Ac Ab, acetylated lysine specific antibody. FIG. 5B shows SVGLL4-K225 is the major VGLL4 acetylation site. VGLL4-GFP was overexpressed in HEK293T cells in the presence of p300, immunoprecipitated with GFP antibody, and analyzed by mass spectrometry. The area of the circles below the solid black horizontal line is proportional to the fraction of peptides detected that contain the acetyl-lysine residue indicated by the corresponding number. T1 and T2 represent the two TDU domains of VGLL4. FIG. 5C shows VGLL4 K225R mutation decreased VGLL4 acetylation. Wild-type (WT) or K255R mutated (R) VGLL4-GFP were co-expressed in HEK293T with p300, as indicated. VGLL4-GFP acetylation was detected by IP-western. FIG. 5D depicts the alignment of TDU domains from different proteins (top group) or from the first TDU domain of VGLL4 from different species (bottom group). The lysine residue (K) aligned with K225 of human VGLL4 (bottom of figure) are depicted with an arrow. This residue is conserved in vertebrate VGLL4 but is not conserved across TDU domains. FIGS. 5E-5F shows VGLL4 K225 acetylation decreased VGLL4-TEAD1 interaction in vitro. Interaction between recombinant His-TEAD1[211-427] and synthetic, unacetylated or K225-acetylated VGLL4 TDU domain peptides was detected by IP-western (FIG. 5E) or by a nanoscale photonic interaction assay (FIG. 5F). FIG. 5G shows VGLL4[R] increased VGLL4-TEAD1 and decreased YAP-TEAD1 interaction in cultured cells. TEAD1^{fb} and VGLL4-GFP expression plasmids were co-transfected into 293T cells. TEAD1 Co-IP was carried out using Flag antibody. FIG. 5H shows the effect of p300 on YAP-TEAD1 transcriptional activity. 293T cells were co-transfected with the indicated plasmids plus pRL-TK. Twenty-four (24) hours after transfection; cells were collected for luciferase activity measurement. Firefly luciferase activity was normalized to Renilla luciferase. *, P<0.05. n=4. FIGS. 51-5J show the effect of VGLL4 acetylation on VGLL4-TEAD1 interaction in NRVM. The proximity ligation assay (PLA) was used to detect endogenous VGLL4-TEAD1 interaction in cultured NRVMs. FIG. 5I shows representative images. FIG. 5J shows the quantification of TEAD1-VGLL4 interaction events in the nucleus. Each dot (see white arrow, FIG. SI) was counted as an interaction event. Bar=20 μm. *, P<0.05, n=3. FIG. 5K shows endogenous levels of mVGLL4 (murine VGLL4) and mVGLL4-K216Ac (which corresponds to human K225Ac) in P6 and P60 heart. Hearts were lysed with denaturing buffer containing SDS (2%) and 100 μg total protein was immunoblotted for total VGLL4 or VGLL4-K216Ac. Fold change of protein levels between P60 and P6 was determined by densitometry. FIG. 5L shows endogenous levels of VGLL4 and VGLL4-K225Ac in human left ventricular myocardium, obtained from unused transplant donor hearts of the indicated ages.

FIGS. 6A-6G depict the analysis of VGLL4 acetylation and the effect on TEAD1 interaction. FIG. 6A depicts the sequence of synthesized VGLL4 TDU domain peptide. The underlined characters indicate V5 peptide sequence. A black arrow pointing up to the acetylated lysine is shown. FIG. 6B shows the results of an experiment where TEAD1 YBD domain (residues 211-427) were fused to His tag, and expressed in E. coli. Soluble proteins were run through Ni resin to purify TEAD1-YBD-His(T-YBD-His). FPLC peaks are labeled with number. The elution volume for peaks 1, 2, 3, 4, 5, 6, is 8.8, 10.6, 15, 17, 20, 21 ml, respectively. Peak “N” not included in the western blot. FIG. 6C shows a Commassie blue staining and western blot. Arrow indicates the T-YBD-His protein. His tag antibody was used to detect His-TEAD1 in the western blot. Peak 3 was run in two lanes, lane 5 and lane 9. In lane 5, samples from peak 3 were diluted 10 times. FIG. 6D shows the validation of TEAD1 and VGLL4 antibodies for immunofluorescence staining. NRVMs were fixed with PFA and stained with the indicated antibodies. TNNI3 was used as a cardiomyocyte marker. Bar=20 μm. FIG. 6E shows the validation of VGLL4-K225Ac antibody. Acetylated or non-acetylated synthetic VGLL4 peptides were bound to nitrocellulose membranes and then probed with antibody directed against total or K225Ac VGLL4. Bound antibody was visualized with HRP-conjugated secondary antibody. FIG. 6F shows the validation of VGLL4-K255Ac antibody in cell lysates. 293T cells were co-transfected with p300 and VGLL4 or VGLL4[R] expression constructs. Lysates were immunoblotted with total VGLL4 and VGLL4-K225-Ac antibodies. FIG. 6G shows the expression of p300 in neonatal and adult mouse heart.

FIGS. 7A-7H show that VGLL4 overexpression decreased TEAD1 stability. FIG. 7A is an immunoblot showing VGLL4 overexpression decreased TEAD1 protein level. Different doses of TEAD1 plasmids (indicated in μg) were co-transfected with 1.6 μg VGLL4-GFP plasmid. Cells were collected for western blot 24 hours after transfection. FIGS. 7B-7C show the generation and validation of TEAD1-Dendra2 construct. Tead1-Dendra2 plasmid was transfected into 293T cells. Western blot confirmed expression of TEAD1-Dendra2 fusion protein (FIG. 7B). TEAD1-Dendra2 merge fusion protein was green before illumination with 405 nm light. After 30 seconds of illumination, a fraction of TEAD1-Dendra2 exhibited red fluorescence. FIGS. 7D-7E show Dox inducible expression of VGLL4 caused TEAD1-Dendra2 degradation. pTEAD1-Dendra2 and pEFla-rtTA were co-transfected into 293T cells along with pTetO empty vector (upper panel) or pTetO::HA-VGLL4 (lower panel). Twenty-four (24) hours after transfection, Dox was added. Cells were analyzed at the indicated time points. FIG. 7D is an immunoblot depicting TEAD1-Dendra2 protein levels. FIG. 7E is a graph showing the quantification of TEAD1-Dendra2 protein levels in FIG. 7D. *P<0.05. n=3. FIG. 7F shows time lapse imaging of TEAD1-Dendra2 or Dendra2 proteins. Indicated plasmids were transfected into 293T cells. Twenty-four (24) hours later, cells were treated with Dox. Four hours later, Dendra2 was photoconverted with 405 nm light. Relative red fluorescence intensity (RFI) was monitored for 20 minutes by taking one image per minute. n=6. Experiment is representative of three independent replicates. FIG. 7G depicts the results of a dual luciferase assay of YAP-TEAD1 transcriptional activity. 293T cells were co-transfected with YAP[S127A], EF1a::rtTA, 8xGIITC-luciferase, pRL-TK internal control, and either TetO empty vector or TetO-VGLL4 as indicated. E64 was added as indicated. Twenty-four (24) hours after transfection, cells were treated with Dox for the indicated number of hours, when cell extracts were analyzed for Firefly and Renilla luciferase activity. Relative luciferase activity was the ratio of Firefly to Renilla luciferase, normalized to empty vector at time 0. *, P<0.05. N=4. FIG. 7H shows a model of VGLL4 regulation of YAP-TEAD1 activity. In the absence of VGLL4, YAP binds to TEAD1 to activate target gene expression. VGLL4 overexpression suppressed YAP-TEAD1 activity by both inhibiting TEAD1 transcriptional activity (i) and promoting TEAD1 degradation (ii).

FIGS. 8A-8D shows that VGLL4 induces TEAD1 degradation. FIG. 8A shows a schematic view of the Doxycycline (Dox) inducible HA-VGLL4 expression system. HA-VGLL4 was cloned downstream of TetO promoter. pEF1α::rtTA was used to express rtTA. The expression of HA-VGLL4 will be activated in the presence of both Dox and rtTA. FIG. 8B shows the validation of Dox inducible expression of HA-VGLL4. 293T cells were co-transfected with pEF1α::rtTA and pTetO::HA-Vgll4. Twenty-four (24) hours after transfection, cells were treated with 1 mg/ml Dox for different hours. Immunoblotting was carried out to detect the expression of HA-VGLL4. FIG. 8C shows the quantification of Tead1-Dendra2 mRNA level following VGLL4 induction. Mouse Tead1 specific primers were used to measure relative Tead1-Dendra2 mRNA level by qRT-PCR. FIG. 8D depicts an immunoblot showing TEAD1 degradation is dependent on cysteine proteases and is independent of the proteasome. 293T cells were first co-transfected with TEAD1^{fb} and Vgll4-GFP plasmids. 1 day after transfection, cells were treated with indicated inhibitors for 6 hours. Dimethyl sulfoxide (DMSO, 0.1%) was used as control vehicle. β-tubulin was used as loading control.

FIGS. 9A-9J show that abrogation of VGLL4-K225 acetylation unmasked the disruptive effects of VGLL4 on YAP-TEAD interaction and neonatal heart maturation. P1 pups were treated with AAV9.VGLL4, AAV9.VGLL4[R] (containing the K225R mutation), or AAV.GFP. Hearts were examined at P8 or P12, as indicated. FIG. 9A depicts an immunoblot used in an assay of cardiac TEAD1 interacting proteins. TEAD1 and its associated proteins were immunoprecipitated, and indicated proteins were detected by western blotting. Asterisk indicates the VGLL4-GFP band. FIG. 9B shows endogenous p300 interacts with and acetylates VGLL4 in the neonatal heart. AAV9.GFP, AAV9.VGLL4-GFP, or AAV9.VGLL4[R]-GFP were administered to P1 mouse pups. p300 was immunoprecipitated from P8 heart extracts and probed with indicated antibodies. K—Ac Ab, acetyl-lysine specific antibody. Asterisk indicates acetylated VGLL4-GFP. Arrowhead indicates VGLL4-GFP band, which runs just above immunoglobulin heavy chain. FIGS. 9C-9D depict the echocardiographic measurement of LV systolic function (fractional shortening, FS) and diastolic left ventricular wall thickness at P8. *, P<0.05 compared to GFP control. n=4. FIG. 9E shows whole mount (upper panels) and H&E stained (lower panels) short axis sections of AAV-transduced hearts at P12. Bar=2 mm. FIG. 9F shows the quantification of heart to body weight ratio of AAV-transduced hearts at P8 or P12. FIGS. 9G-9H depicts histology of cardiac fibrosis, visualized by pirosirius red/fast green staining. FIG. 9G shows representative images and quantification (FIG. 9H). Bar=1 mm. *, P<0.05. n=3. FIGS. 9I-9J shows the quantification of qRT-PCR measurement of heart failure marker gene transcripts Myh6 and Nppa. Levels were normalized to GAPDH and expressed relative to the AAV9.GFP control group. *, P<0.05. n=4

FIGS. 10A-10C show that VGLL4 acetylation regulates heart growth and function. FIG. 10A shows results indicating that in the adult heart p300 does not interact with VGLL4. AAV9.GFP (GFP) and AAV9.VGLL4-GFP (V) were delivered into the P1 mouse pups, respectively. At P60, hearts were collected for p300 Co-IP assay. Arrow indicates non-specific IgG band. VGLL4-GFP did not detectably co-immunoprecipitate with p300 in adult heart (shown), whereas it did in the neonatal heart (FIG. 10B). FIGS. 10B-10C show quantification of heart and body weight after transduction with the indicated virus at P1. *, P<0.05 com-pared to control (GFP) at the same age. P8, N=3. P12, N=4.

FIGS. 11A-11D shows cardiomyocyte formation and loss in hearts expressing acetylation-defective VGLL4. FIG. 11A shows the results of VGLL4[R] overexpression, which caused heart failure without affecting cardiomyocyte apoptosis. Mouse pups were transduced with indicated virus at P1, and hearts were collected for analysis at P12. TUNEL assay on heart sections did not reveal significant cardiomyocyte apoptosis. The rectangle and inset shows a TUNEL+ non-cardiomyocyte. Bar=100 μm. FIGS. 11B-11D shows the titration of AAV9.cTNT::Cre in neonatal Rosa26^{confetti} mice (supporting FIG. 12). Different doses of AAV9.cTNT::Cre (AAV genome copies per gram body weight) were administered to Pb Rosa26^{confetti/+} mice. Hearts were collected for analysis at P8. Cre recombination activated nuclear GFP, YFP and RFP expression. FIG. 11B shows representative immunofluorescent images of heart cryosections. Bar=500 μm. FIGS. 11C-11D shows the quantification of clones. 1×10⁸ GC/g is the dose that was selected for further experiments.

FIGS. 12A-12J show that acetylation-deficient VGLL4[R] decreased cardiomyocyte proliferation and survival. FIGS. 12A-12J depict the results of experiments in which AAV9.GFP, AAV9.VGLL4, or AAV9.VGLL4[R] was delivered to P1 pups and hearts were examined at P8. FIGS. 12A-12B show the measurement of CM necrosis. Rosa26^{mTmG} (membrane localized RFP) P1 pups were treated with AAV9. Anti-myosin antibody MF20 was injected into mice at P7. At P8, mice were collected and intracellular MF20 antibody was detected by immunofluorescent staining. FIG. 12A shows representative images. FIG. 12B shows quantification of the experiment described above. *, P<0.05. n=3. FIGS. 12C-12D show the measurement of CM proliferation using pH3 immunofluorescence staining. FIG. 12C shows representative images with boxed regions magnified in insets, Bar=50 μm. FIG. 12D shows the quantification of pH3+ CMs. FIGS. 12E-12F depict a clonal assay for CM proliferation. Confetti P1 pups were treated with mosaic dose of AAV9.Cre to label individual CMs, in addition to treatment with VGLL4 or control AAVs at the usual dose, which transduces nearly all CMs. Hearts were examined at P8. FIG. 12E depicts representative images, with boxed regions magnified in insets. Bar=50 μm. FIG. 12F shows the quantification of clusters of adjacent, labeled CMs containing one color (monochromatic, potentially arising from proliferation) or two colors (bichromatic, arising from adjacent labeling events). Bar=50 *, P<0.05. n=4. FIG. 12G shows qRT-PCR quantification of relative levels of YAP-TEAD canonical targets gene transcripts Aurka, Cdc20, and Ctgf in P12 heart. *, P<0.05. n=3. FIG. 12F shows the measurement of cardiomyocyte cross-sectional area. CMs were outlined by wheat germ agglutinin staining. FIG. 12H shows representative images of CMs. FIG. 12I shows quantification of CM cross sectional area. *, P<0.05. n=3. FIG. 12J depicts a model of VGLL4 regulation of heart growth. In normal newborn heart, predominant YAP-TEAD1 stimulates CM proliferation. The interaction between VGLL4 and TEAD1 is blunted by p300-mediated VGLL4 acetylation. Inhibition of VGLL4 acetylation, as in the K225R mutant, suppresses cardiac growth by both inhibiting YAP-TEAD1 interaction and decreasing TEAD1 stability.

FIGS. 13A-13C depict AAV9-hYAP and YAP synthetic modified RNA (modRNA) in cardiac repair. FIG. 13A depicts an assay with AAV9-hYAP in which AAV9-hYAP improved cardiac outcome after ischemia and reperfusion (I/R). Echocardiography measurement of heart function is plotted in the graph (left) and measurements are provided in the chart (right). Heart function was measured at 1 week, 4 weeks, and 8 weeks after I/R. FIG. 13B depicts workflow of ischemia and reperfusion (I/R) model for AAV9-hYAP. W, week. To cause ischemia, left anterior descending artery was ligated for half an hour (left). Workflow of the long term study is provided (right). FIG. 13C depicts workflows of ischemia and reperfusion (I/R) model for YAP modRNA and/or E64d. A schematic depicting the procedures performed on the heart and a listing of test groups (left). To cause ischemia, left anterior descending artery was ligated for half an hour. YAP modRNA and/or E64d was directly delivered into myocardium during ischemia. Workflows of long term and short term studies are provided (right). W, week. Short term study: 4 animals for each group. Long term study: 6 animals for each group. In the short term study, MF20 antibody is delivered to the mice one day before heart collection.

FIG. 14 (FIG. 14A, FIG. 14B, and FIG. 14C) show YAP containing an activating S127A mutation (aYAP) modRNA was successfully expressed in myocardium. FIG. 14A shows the experimental design. I/R and modRNA injection was performed as described in the material and methods. Short term and long term studies were performed to assess the effects of aYAP modRNA. In the long term study, heart function was measured by echocardiography at 1 and 4 weeks after IR. 3 months after IR and modRNA treatment, hearts were analyzed for size and histology. FIG. 14B is a graph that provides YAP mRNA level measured by qRT-PCR. *P<0.05, n=3. Hearts were collected 2 days after I/R and modRNA injection. Heart apex was used for RNA isolation and qRT-PCR. FIG. 14C is an immunoblot showing the expression of YAP protein. aYAP was fused to a 3xFlag tag. Flag antibody immunoblot showed the expression of aYAP in the aYAP modRNA-treated group but not in the Luci-treated group.

FIG. 15 (FIG. 15A, FIG. 15B, and FIG. 15C) show aYAP modRNA treatment before reperfusion reduced cardiac inflammation. FIG. 15A shows the morphology of hearts receiving different treatment. Bar=5 mm. Fluorescent red microbeads were used to indicate blow flow. Dark regions with no red fluorescence indicate area at risk. Hearts were collected at 2 days after IR. FIG. 15B is a graph that provides heart weight to tibia length ratio. *, P<0.01. N=3. FIG. 15C shows representative images of hematoxylin and eosin-stained heart sections. In Luci+IR and aYAP+IR group, typical images from ischemic regions are shown, with boxed areas in upper row images magnified in the lower row. Bar=50 μm. FIG. 16 (FIG. 16A, FIG. 16B, and FIG. 16C) shows aYAP modRNA treatment at time of reperfusion improved heart function and suppresses cardiac hypertrophy in a murine I/R model. FIG. 16A shows the ejection fraction (EF) measured by echocardiography. B-mode echocardiography was used to measure EF at 1 week and 4 weeks after I/R. In each group, EF was analyzed by paired T-test. FIG. 16B is an image that shows heart morphology. Hearts were collected 3 months after I/R. Bar=5 mm. Fluorescent red microbeads were used to label tissue perfused during ischemic period; dark regions without red fluorescence indicate area at risk. FIG. 16C is a graph that provides heart weight to tibia length ratio. **, P<0.01. N=4.

### DETAILED DESCRIPTION OF THE INVENTION

The invention features compositions comprising a modified RNA encoding a YAP polypeptide and methods of using the compositions for transient expression of a YAP polypeptide to promote cardiac repair in a subject in need thereof. In particular embodiments, a modified RNA encoding a YAP polypeptide is administered in combination with E64d.

The invention is based, at least in part, on the discovery that transient expression of YAP is sufficient to promote regeneration in cardiac muscle. Binding of the transcriptional co-activator YAP with the transcription factor TEAD stimulates growth of the heart and other organs. YAP overexpression potently stimulates fetal cardiomyocyte (CM) proliferation, but YAP's mitogenic potency declines postnatally. While investigating factors that limit YAP's postnatal mitogenic activity, the cardiac myocyte (CM)-enriched TEAD1 binding protein VGLL4 inhibits CM proliferation by inhibiting TEAD1-YAP interaction and by targeting TEAD1 for degradation. Importantly, VGLL4 acetylation at lysine 225 negatively regulated its binding to TEAD1. This developmentally regulated acetylation event critically governs postnatal heart growth, since overexpression of an acetylation-refractory VGLL4 mutant enhanced TEAD1 degradation, limited neonatal CM proliferation, and caused CM necrosis. The results provided herein below defines an acetylation-mediated, VGLL4-dependent switch that regulates TEAD stability and YAP-TEAD activity. Accordingly, the invention provides compositions and methods for transiently expressing YAP in a CM, thereby promoting cardiac regeneration in the tissue.

### Yes-Associated Protein (YAP)

The transcriptional co-activator YAP (Yes-associated protein) is a key driver of organ growth. YAP binds to TEA-domain (TEAD)-containing transcription factors (TEAD1-4) to activate transcription of cell cycle and cell survival genes and thereby promotes organ growth. The potent growth promoting activity of the YAP-TEAD complex is closely regulated through incompletely understood signaling pathways. Among the most studied regulatory mechanisms is the Hippo kinase cascade, which phosphorylates YAP, leading to its nuclear exclusion. Both YAP and its regulation by the Hippo kinase cascade have been shown to be essential for normal heart development. YAP was necessary for fetal CM proliferation, and its activation through overexpression or Hippo inhibition was sufficient to drive massive fetal cardiac overgrowth. YAP activation likewise stimulated neonatal as well as adult CM proliferation, but the level of CM cell cycle activity achieved diminished with postnatal age (Lin et al., 2014; Xin et al., 2013; Heallen et al., 2013). These data show that regulation of YAP activity is crucial for normal cardiac growth control. Moreover, they suggest that unknown mechanisms suppress YAP mitogenic activity in the postnatal heart.

In addition to the Hippo kinase pathway, Hippo-independent YAP regulatory mechanisms also exist. For example, α-catenin, a cellular adhesion molecule, binds YAP under high cell density conditions, promoting its cytoplasmic sequestration by limiting its dephosphorylation (Schlegelmilch et al., 2011; Li et al., 2014). Recently, a new level of YAP-TEAD regulation was described. In Drosophila, the orthologs of YAP and TEAD are named Yorkie (Yki) and Scalloped (Sd), respectively. The protein Tgi was discovered in Drosophila screens for Yki-Sd antagonists (Koontz et al., 2013). Tgi contains two TEAD-binding regions, named Tondu (TDU) domains, and competes with Yki for Sd binding. By reducing Yki-Sd activity and the transcription of Yki-Sd target genes, Tgi inhibited growth. In mammals, there are four TDU domain-containing proteins, vestigial-like 1 to 4 (VGLL1-VGLL4), with VGLL4 being the most closely related to Tgi. Massive liver overgrowth driven by YAP was suppressed by VGLL4 (Koontz et al., 2013), indicating that VGLL4 is a potent inhibitor of YAP in mammalian cells.

Interestingly, profiling of VGLL4 across mouse tissues showed that it was most highly expressed in heart (Chen et al., 2004), suggesting a potential role for VGLL4 in suppression of postnatal cardiac YAP activity. As reported in detail below, the function of VGLL4 in regulating cardiac YAP-TEAD activity and neonatal cardiac growth and function was defined. VGLL4 regulated both TEAD stability and its interaction with YAP. Moreover, VGLL4 acetylation at a key residue within the TDU domain regulated its binding to TEAD, revealing a novel YAP-TEAD regulatory mechanism. Acetylation of VGLL4 in neonatal heart was essential to limit its activity and thereby permit normal heart growth and function.

### Methods for Transient YAP Therapy

Synthetic, modified-RNAs encoding a YAP protein can be used to express the YAP protein in a target tissue (e.g., cardiac tissue) or organ (e.g., heart) by administration of a synthetic, modified-RNA composition to an individual or in alternative embodiments, by contacting cells (e.g., cardiac myocytes) with a synthetic, modified-RNA ex vivo, and then administering such cells to a subject. In one aspect, cells can be transfected with a modified RNA to express a YAP protein using an ex vivo approach in which cells are removed from a patient, transfected by e.g., electroporation or lipofection, and re-introduced to the patient.

In various embodiments, the level, expression, or activity of YAP in a cell (e.g., cardiac) in vivo is transiently increased. In various embodiments, this is accomplished by administering an agent (e.g., a synthetic, modified-RNA to express a YAP protein, E64d). In various embodiments, the level, expression, or activity of YAP in a cell is increased for about 3 months, 2 months, 1 month or less after injury or damage to the heart (e.g., myocardial infarction, congestive heart failure, cardiomyopathy, myocardial infarction, tissue ischemia, cardiac ischemia, planned cardiac surgery, stent placement). In various embodiments, the level, expression, or activity of YAP in a cell is increased for about 4 weeks, 3 weeks, 2 weeks, 1 week or less after injury or damage to the heart. In various embodiments, the level, expression, or activity of YAP in a cell is increased for about 7 days, 6 days, 5 days, 4 days, 3 days, 2 days, 1 day or less after injury or damage to the heart. In various embodiments, the level, expression, or activity of YAP in a cell is increased for about 24, 23, 22, 21, 20, 19, 18, 17, 16, 15, 14, 13, 12, 11, 10, 9, 8, 7, 6, 5, 4, 3, 2, 1 hr. or less after injury or damage to the heart. In various embodiments, the level, expression, or activity of YAP in a cell is increased for about 60, 45, 30, 15, 10, 9, 8, 7, 6, 5, 4, 3, 2, 1 min. or less after injury or damage to the heart. In various embodiments, the increase in the level, expression, or activity of YAP in a cell is not constitutive.

In various embodiments, the agent is administered within about 3 months, 2 months, or 1 month of injury or damage to the heart (e.g., myocardial infarction, congestive heart failure, cardiomyopathy, myocardial infarction, tissue ischemia, cardiac ischemia). In various embodiments, the agent is administered within about 4 weeks, 3 weeks, 2 weeks, 1 week or less of injury or damage to the heart. In various embodiments, the agent is administered within about 7 days, 6 days, 5 days, 4 days, 3 days, 2 days, 1 day or less of injury or damage to the heart. In various embodiments, the agent is administered within about 24, 23, 22, 21, 20, 19, 18, 17, 16, 15, 14, 13, 12, 11, 10, 9, 8, 7, 6, 5, 4, 3, 2, 1 hr. or less of injury or damage to the heart. In various embodiments, the agent is administered within about 60, 45, 30, 15, 10, 9, 8, 7 6, 5, 4, 3, 2, 1 min. or less of injury or damage to the heart. In various embodiments, the agent is administered immediately after or at the time of injury or damage to the heart.

In various embodiments, the agent is administered about 3 months, 2 months, 1 month or less prior to a cardiac surgery (e.g., planned cardiac surgery, stent placement, etc.). In various embodiments, the agent is administered about 4 weeks, 3 weeks, 2 weeks, 1 week or less prior to cardiac surgery. In various embodiments, the agent is administered about 24, 23, 22, 21, 20, 19, 18, 17, 16, 15, 14, 13, 12, 11, 10, 9, 8, 7, 6, 5, 4, 3, 2, 1 hr. or less prior to cardiac surgery. In various embodiments, the agent is administered within about 60, 45, 30, 15, 10, 9, 8, 7 6, 5, 4, 3, 2, 1 min. or less prior to cardiac surgery. In various embodiments, the agent is administered immediately prior to or at the time of cardiac surgery.

Synthetic, modified-RNA to express a YAP protein can be administered to a target tissue in vivo as a single dose or in multiple doses (e.g., sequentially). The expression desired for YAP protein in vivo can be tailored by altering the frequency of administration and/or the amount of the synthetic, modified-RNA administered. The methods and compositions described herein permit the in vivo protein expression of YAP protein to be tuned to a desired level by varying the amount of YAP synthetic, modified-RNA transfected. Because the YAP synthetic, modified-RNA is administered to a target tissue (e.g. cardiac) in vivo is degraded over time one of skill in the art can remove or stop the in vivo protein expression from a synthetic, modified-RNA by halting further transfections and permitting the tissue to degrade the synthetic, modified-RNA over time. The synthetic, modified-RNAs will degrade in a manner similar to cellular mRNAs. One of skill in the art can easily monitor level of in vivo protein expression encoded by a synthetic, modified-RNA using e.g., Western blotting techniques or immunocytochemistry techniques. A synthetic, modified RNA to express YAP can be administered at a frequency and dose to a target tissue in vivo that permits a desired level of in vivo protein expression. As disclosed herein in the Examples, the amount of MOD-RNA administered in vivo determines the amount of in vivo protein expression, and therefore the amount of protein expressed can be controlled based on the amount of modRNA administered to the target tissue (e.g., cardiac) in vivo.

### Synthetic Modified RNAs Expressing YAP Polypeptides

The invention provides methods for YAP protein expression in vivo in a tissue, e.g., in a heart tissue, or a cardiomyocyte by contacting a population of heart cells, e.g., cardiomyocytes with a composition comprising at least one synthetic modified RNA (MOD-RNA) encoding a polypeptide.

Synthetic modified RNA's for use in the compositions, methods and kits as disclosed herein are described in U.S. Provisional Application 61/387,220, filed Sep. 28, 2010, and U.S. Provisional Application 61/325,003, filed: Apr. 16, 2010, both of which are incorporated herein in their entirety by reference.

As used herein, the term “synthetic, modified RNA” (also referred herein as MOD-RNA) refers to a nucleic acid molecule encoding a factor, such as a polypeptide, to be expressed in a host cell, which comprises at least one modified nucleoside and has at least the following characteristics as the term is used herein: (i) it can be generated by in vitro transcription or chemical synthesis and is not isolated from a cell; (ii) it is translatable in vivo in a mammalian (and preferably human) cell; and (iii) it does not provoke or provokes a significantly reduced innate immune response or interferon response in a cell to which it is introduced or contacted relative to a synthetic, non-modified RNA of the same sequence. A synthetic, modified-RNA as described herein permits repeated transfections in a target cell or tissue in vivo; that is, a cell or cell population transfected in vivo with a synthetic, modified-RNA molecule as described herein tolerates repeated transfection with such synthetic, modified-RNA without significant induction of an innate immune response or interferon response. These three primary criteria for a synthetic, modified RNA molecule described above are described in greater detail below.

First, the synthetic, modified-RNA must be able to be generated by in vitro transcription of a DNA template. Methods for generating templates are well known to those of skill in the art using standard molecular cloning techniques. An additional approach to the assembly of DNA templates that does not rely upon the presence of restriction endonuclease cleavage sites is also described herein (termed “splint-mediated ligation”). The transcribed, synthetic, modified-RNA polymer can be modified further post-transcription, e.g., by adding a cap or other functional group.

To be suitable for in vitro transcription, the modified nucleoside(s) must be recognized as substrates by at least one RNA polymerase enzyme expressed by the tissue or cell which is transfected with the MOD-RNA. Generally, RNA polymerase enzymes can tolerate a range of nucleoside base modifications, at least in part because the naturally occurring G, A, U, and C nucleoside bases differ from each other quite significantly. Thus, the structure of a modified nucleoside base for use in generating the synthetic, modified-RNAs described herein can generally vary more than the sugar-phosphate moieties of the modified nucleoside. That said, ribose and phosphate-modified nucleosides or nucleoside analogs are known in the art that permit transcription by RNA polymerases. In some embodiments of the aspects described herein, the RNA polymerase is a phage RNA polymerase. The modified nucleotides pseudouridine, m5U, s2U, m6A, and m5C are known to be compatible with transcription using phage RNA polymerases. Polymerases that accept modified nucleosides are known to those of skill in the art.

It is also contemplated that modified polymerases can be used to generate synthetic, modified-RNAs, as described herein. Thus, for example, a polymerase that tolerates or accepts a particular modified nucleoside as a substrate can be used to generate a synthetic, modified-RNA including that modified nucleoside.

Second, the synthetic, modified-RNA must be translatable in vivo by the translation machinery of a eukaryotic, preferably mammalian, and more preferably, human cell in vivo. Translation in vivo generally requires at least a ribosome binding site, a methionine start codon, and an open reading frame encoding a polypeptide. Preferably, the synthetic, modified-RNA also comprises a 5′ cap, a stop codon, a Kozak sequence, and a polyA tail. In addition, mRNAs in a eukaryotic cell are regulated by degradation, thus a synthetic, modified-RNA as described herein can be further modified to extend its half-life in the cell by incorporating modifications to reduce the rate of RNA degradation (e.g., by increasing serum stability of a synthetic, modified-RNA).

Nucleoside modifications can interfere with translation. To the extent that a given modification interferes with translation, those modifications are not encompassed by the synthetic, modified-RNA as described herein. One can test a synthetic, modified-RNA for its ability to undergo translation and translation efficiency using an in vivo translation assay (e.g., using a MOD-RNA encoding a cre recombinase gene in an in vivo mouse cre model assay, or MOD-RNA encoding luciferase and detecting expression in vivo using a bioluminescence assay of the translated protein) and detecting the amount of the polypeptide produced using SDS-PAGE, Western blot, or immunochemistry, bioluminescence assays, etc. The translation of a synthetic, modified-RNA comprising a candidate modification is compared to the translation of an RNA lacking the candidate modification, such that if the translation of the synthetic, modified-RNA having the candidate modification remains the same or is increased then the candidate modification is contemplated for use with the compositions and methods described herein. It is noted that fluoro-modified nucleosides are generally not translatable and can be used herein as a negative control for an in vitro translation assay.

Third, the synthetic, modified-RNA provokes a reduced (or absent) innate immune response in vivo or reduced interferon response in vivo by the transfected tissue or cell population. mRNA produced in eukaryotic cells, e.g., mammalian or human cells, is heavily modified, the modifications permitting the cell to detect RNA not produced by that cell. The cell responds by shutting down translation or otherwise initiating an innate immune or interferon response. Thus, to the extent that an exogenously added RNA can be modified to mimic the modifications occurring in the endogenous RNAs produced by a target cell, the exogenous RNA can avoid at least part of the target cell's defense against foreign nucleic acids. Thus, in some embodiments, synthetic, modified-RNAs as described herein include in vitro transcribed RNAs including modifications as found in eukaryotic/mammalian/human RNA in vivo. Other modifications that mimic such naturally occurring modifications can also be helpful in producing a synthetic, modified-RNA molecule that will be tolerated by a cell. With this as a background or threshold understanding for the requirements of a synthetic, modified-RNA, the various modifications contemplated or useful in the synthetic, modified-RNAs described herein are discussed further herein below.

### RNA Modifications

The invention provides a modified RNA encoding a YAP protein for transient expression of YAP in a cell of a cardiac tissue (e.g., cardiac myocyte) before, during, or after an insult to the heart (e.g., coronary occlusion, myocardial infarction, ischemia reperfusion injury, or heart surgery). In some aspects, provided herein are synthetic, modified RNA molecules (MOD-RNAs) encoding YAP polypeptides, wherein the synthetic, modified RNA molecules comprise one or more modifications, such that introducing the synthetic, modified RNA molecules to a cell or tissue in vivo results in a reduced innate immune response of the tissue in vivo relative to a cell contacted with synthetic RNA molecules encoding the polypeptides not comprising said one or more modifications.

The synthetic, modified-RNAs as described herein include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation, dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein. Specific examples of synthetic, modified-RNA compositions useful with the methods described herein include, but are not limited to, RNA molecules containing modified or non-natural internucleoside linkages. Synthetic, modified-RNAs having modified internucleoside linkages include, among others, those that do not have a phosphorus atom in the internucleoside linkage. In other embodiments, the synthetic, modified-RNA has a phosphorus atom in its internucleoside linkage(s).

Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39,464, each of which is herein incorporated by reference in its entirety.

Modified internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts.

Representative U.S. patents that teach the preparation of modified oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, each of which is herein incorporated by reference in its entirety.

Some embodiments of the synthetic, modified-RNAs described herein include nucleic acids with phosphorothioate internucleoside linkages and oligonucleosides with heteroatom internucleoside linkage, and in particular —CH2-NH—CH2-, —CH2-N(CH3)-O—CH2-[known as a methylene (methylimino) or MMI], —CH2-O—N(CH3)-CH2-, —CH2-N(CH3)-N(CH3)-CH2- and —N(CH3)-CH2-CH2-[wherein the native phosphodiester internucleoside linkage is represented as —O—P—O—CH2-] of the above-referenced U.S. Pat. No. 5,489,677, and the amide backbones of the above-referenced U.S. Pat. No. 5,602,240, both of which are herein incorporated by reference in their entirety. In some embodiments, the nucleic acid sequences featured herein have morpholino backbone structures of the above-referenced U.S. Pat. No. 5,034,506, herein incorporated by reference in its entirety.

Synthetic, modified-RNAs described herein can also contain one or more substituted sugar moieties. The nucleic acids featured herein can include one of the following at the 2′ position: H (deoxyribose); OH (ribose); F; O—, S—, or N-alkyl; O—, S—, or N-alkenyl; O—, S— or N-alkynyl; or O-alkyl-O-alkyl, wherein the alkyl, alkenyl and alkynyl can be substituted or unsubstituted C1 to C10 alkyl or C2 to C10 alkenyl and alkynyl. Exemplary modifications include O[(CH2)nO]mCH3, O(CH2).nOCH3, O(CH2)nNH2, O(CH2)nCH3, O(CH2)nONH2, and O(CH2)nON[(CH2)nCH3)]2, where n and m are from 1 to about 10. In some embodiments, synthetic, modified-RNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, a reporter group, an intercalator, a group for improving the pharmacokinetic properties of an RNA, or a group for improving the pharmacodynamic properties of a synthetic, modified-RNA, and other substituents having similar properties. In some embodiments, the modification includes a 2′ methoxyethoxy (2′-O—CH2CH2OCH3, also known as 2′-O-(2-methoxyethyl) or 2′-MOE) (Martin et al., Hely. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, and 2′-dimethylaminoethoxyethoxy (also known in the art as 2′-O-dimethylaminoethoxyethyl or 2′-DMAEOE), i.e., 2′-O—CH2-O—CH2-N(CH2)2.

Other modifications include 2′-methoxy (2′-OCH3), 2′-aminopropoxy (2′-OCH₂CH₂CH₂NH₂) and 2′-fluoro (2′-F). Similar modifications can also be made at other positions on the nucleic acid sequence, particularly the 3′ position of the sugar on the 3′ terminal nucleotide or in 2′-5′ linked nucleotides and the 5′ position of 5′ terminal nucleotide. A synthetic, modified-RNA can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

As non-limiting examples, synthetic, modified-RNAs described herein can include at least one modified nucleoside including a 2′-O-methyl modified nucleoside, a nucleoside comprising a 5′ phosphorothioate group, a 2′-amino-modified nucleoside, 2′-alkyl-modified nucleoside, morpholino nucleoside, a phosphoramidate or a non-natural base comprising nucleoside, or any combination thereof.

In some embodiments of this aspect and all other such aspects described herein, the at least one modified nucleoside is selected from the group consisting of 5-methylcytidine (5mC), N6-methyladenosine (m6A), 3,2′-O-dimethyluridine (m4U), 2-thiouridine (s2U), 2′ fluorouridine, pseudouridine, N1-methyl-pseudouridine, 2′-O-methyluridine (Um), 2′ deoxyuridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m-2,7G), N2,N2,7-trimethylguanosine (m-2,2,7G), and inosine (I).

Alternatively, a synthetic, modified-RNA can comprise at least two modified nucleosides, at least 3, at least 4, at least 5, at least 6, at least 7, at least 8, at least 9, at least 10, at least 15, at least 20 or more, up to the entire length of the oligonucleotide. At a minimum, a synthetic, modified-RNA molecule comprising at least one modified nucleoside comprises a single nucleoside with a modification as described herein. It is not necessary for all positions in a given synthetic, modified-RNA to be uniformly modified, and in fact more than one of the aforementioned modifications can be incorporated in a single synthetic, modified-RNA or even at a single nucleoside within a synthetic, modified-RNA. However, it is preferred, but not absolutely necessary, that each occurrence of a given nucleoside in a molecule is modified (e.g., each cytosine is a modified cytosine e.g., 5mC). However, it is also contemplated that different occurrences of the same nucleoside can be modified in a different way in a given synthetic, modified-RNA molecule (e.g., some cytosines modified as 5mC, others modified as 2′-O-methylcytidine or other cytosine analog). The modifications need not be the same for each of a plurality of modified nucleosides in a synthetic, modified-RNA. Furthermore, in some embodiments of the aspects described herein, a synthetic, modified-RNA comprises at least two different modified nucleosides. In some such preferred embodiments of the aspects described herein, the at least two different modified nucleosides are 5-methylcytidine and pseudouridine. A synthetic, modified-RNA can also contain a mixture of both modified and unmodified nucleosides.

As used herein, “unmodified” or “natural” nucleosides or nucleobases include the purine bases adenine (A) and guanine (G), and the pyrimidine bases thymine (T), cytosine (C) and uracil (U). In some embodiments, a synthetic, modified-RNA comprises at least one nucleoside (“base”) modification or substitution. Modified nucleosides include other synthetic and natural nucleobases such as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyll)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(i sopentyl)adenine, N6 (methyl)adenine, N6, N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)phenthiazin-1-yl, 7-substituted 1-(aza)-2-(thio)-3-(aza)-phenthiazin-1-yl, 7-(aminoalkylhydroxy)-1,3 -(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguani sine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, O6-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Modified nucleosides also include natural bases that comprise conjugated moieties, e.g. a ligand. As discussed herein above, the RNA containing the modified nucleosides must be translatable in a host cell (i.e., does not prevent translation of the polypeptide encoded by the modified RNA). For example, transcripts containing s2U and m6A are translated poorly in rabbit reticulocyte lysates, while pseudouridine, m5U, and m5C are compatible with efficient translation. In addition, it is known in the art that 2′-fluoro-modified bases useful for increasing nuclease resistance of a transcript, leads to very inefficient translation. Translation can be assayed by one of ordinary skill in the art using e.g., a rabbit reticulocyte lysate translation assay.

Further modified nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in Int. Appl. No. PCT/US09/038,425, filed Mar. 26, 2009; those disclosed in The Concise Encyclopedia Of Polymer Science And Engineering, pages 858-859, Kroschwitz, J. L, ed. John Wiley & Sons, 1990, and those disclosed by Englisch et al., Angewandte Chemie, International Edition, 1991, 30, 613.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety, and U.S. Pat. No. 5,750,692, also herein incorporated by reference in its entirety.

Another modification for use with the synthetic, modified-RNAs described herein involves chemically linking to the RNA one or more ligands, moieties or conjugates that enhance the activity, cellular distribution or cellular uptake of the RNA. Ligands can be particularly useful where, for example, a synthetic, modified-RNA is administered in vivo. Such moieties include but are not limited to lipid moieties such as a cholesterol moiety (Letsinger et al., Proc. Natl. Acid. Sci. USA, 1989, 86: 6553-6556, herein incorporated by reference in its entirety), cholic acid (Manoharan et al., Biorg. Med. Chem. Let., 1994, 4:1053-1060, herein incorporated by reference in its entirety), a thioether, e.g., beryl-5-tritylthiol (Manoharan et al., Ann. N.Y. Acad. Sci., 1992, 660:306-309; Manoharan et al., Biorg. Med. Chem. Let., 1993, 3:2765-2770, each of which is herein incorporated by reference in its entirety), a thiocholesterol (Oberhauser et al., Nucl. Acids Res., 1992, 20:533-538, herein incorporated by reference in its entirety), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54, each of which is herein incorporated by reference in its entirety), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783, each of which is herein incorporated by reference in its entirety), a polyamine or a polyethylene glycol chain (Manoharan et al., Nucleosides & Nucleotides, 1995, 14:969-973, herein incorporated by reference in its entirety), or adamantane acetic acid (Manoharan et al., Tetrahedron Lett., 1995, 36:3651-3654, herein incorporated by reference in its entirety), a palmityl moiety (Mishra et al., Biochim. Biophys. Acta, 1995, 1264:229-237, herein incorporated by reference in its entirety), or an octadecylamine or hexylamino-carbonyloxycholesterol moiety (Crooke et al., J. Pharmacol. Exp. Ther., 1996, 277:923-937, herein incorporated by reference in its entirety).

The synthetic, modified-RNAs described herein can further comprise a 5′ cap. In some embodiments of the aspects described herein, the synthetic, modified-RNAs comprise a 5′ cap comprising a modified guanine nucleotide that is linked to the 5′ end of an RNA molecule using a 5′-5′ triphosphate linkage. As used herein, the term “5′ cap” is also intended to encompass other 5′ cap analogs including, e.g., 5′ diguanosine cap, tetraphosphate cap analogs having a methylene-bis(phosphonate) moiety (see e.g., Rydzik, A M et al., (2009) Org Biomol Chem 7(22):4763-76), dinucleotide cap analogs having a phosphorothioate modification (see e.g., Kowalska, J. et al., (2008) RNA 14(6):1119-1131), cap analogs having a sulfur substitution for a non-bridging oxygen (see e.g., Grudzien-Nogalska, E. et al., (2007) RNA 13(10): 1745-1755), N7-benzylated dinucleoside tetraphosphate analogs (see e.g., Grudzien, E. et al., (2004) RNA 10(9): 1479-1487), or anti-reverse cap analogs (see e.g., Jemielity, J. et al., (2003) RNA 9(9): 1108-1122 and Stepinski, J. et al., (2001) RNA 7(10):1486-1495). In one such embodiment, the 5′ cap analog is a 5′ diguanosine cap. In some embodiments, the synthetic, modified RNA does not comprise a 5′ triphosphate.

The 5′ cap is important for recognition and attachment of an mRNA to a ribosome to initiate translation. The 5′ cap also protects the synthetic, modified-RNA from 5′ exonuclease mediated degradation. It is not an absolute requirement that a synthetic, modified-RNA comprise a 5′ cap, and thus in other embodiments the synthetic, modified-RNAs lack a 5′ cap. However, due to the longer half-life of synthetic, modified-RNAs comprising a 5′ cap and the increased efficiency of translation, synthetic, modified-RNAs comprising a 5′ cap are preferred herein.

The synthetic, modified-RNAs described herein can further comprise a 5′ and/or 3′ untranslated region (UTR). Untranslated regions are regions of the RNA before the start codon (5′) and after the stop codon (3′), and are therefore not translated by the translation machinery. Modification of an RNA molecule with one or more untranslated regions can improve the stability of an mRNA, since the untranslated regions can interfere with ribonucleases and other proteins involved in RNA degradation. In addition, modification of an RNA with a 5′ and/or 3′ untranslated region can enhance translational efficiency by binding proteins that alter ribosome binding to an mRNA. Modification of an RNA with a 3′

UTR can be used to maintain a cytoplasmic localization of the RNA, permitting translation to occur in the cytoplasm of the cell. In one embodiment, the synthetic, modified-RNAs described herein do not comprise a 5′ or 3′ UTR. In another embodiment, the synthetic, modified-RNAs comprise either a 5′ or 3′ UTR. In another embodiment, the synthetic, modified-RNAs described herein comprise both a 5′ and a 3′UTR. In one embodiment, the 5′ and/or 3′ UTR is selected from an mRNA known to have high stability in the cell (e.g., a murine alpha-globin 3′ UTR). In some embodiments, the 5′ UTR, the 3′ UTR, or both comprise one or more modified nucleosides.

In some embodiments, the synthetic, modified-RNAs described herein further comprise a Kozak sequence. The “Kozak sequence” refers to a sequence on eukaryotic mRNA having the consensus (gcc)gccRccAUGG, where R is a purine (adenine or guanine) three bases upstream of the start codon (AUG), which is followed by another ‘G’. The Kozak consensus sequence is recognized by the ribosome to initiate translation of a polypeptide. Typically, initiation occurs at the first AUG codon encountered by the translation machinery that is proximal to the 5′ end of the transcript. However, in some cases, this AUG codon can be bypassed in a process called leaky scanning The presence of a Kozak sequence near the AUG codon will strengthen that codon as the initiating site of translation, such that translation of the correct polypeptide occurs. Furthermore, addition of a Kozak sequence to a synthetic, modified-RNA will promote more efficient translation, even if there is no ambiguity regarding the start codon. Thus, in some embodiments, the synthetic, modified-RNAs described herein further comprise a Kozak consensus sequence at the desired site for initiation of translation to produce the correct length polypeptide. In some such embodiments, the Kozak sequence comprises one or more modified nucleosides.

In some embodiments, the synthetic, modified-RNAs described herein further comprise a “poly (A) tail”, which refers to a 3′ homopolymeric tail of adenine nucleotides, which can vary in length (e.g., at least 5 adenine nucleotides) and can be up to several hundred adenine nucleotides). The inclusion of a 3′ poly(A) tail can protect the synthetic, modified-RNA from degradation in the cell, and also facilitates extra-nuclear localization to enhance translation efficiency. In some embodiments, the poly(A) tail comprises between 1 and 500 adenine nucleotides; in other embodiments the poly(A) tail comprises at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 225, at least 250, at least 275, at least 300, at least 325, at least 350, at least 375, at least 400, at least 425, at least 450, at least 475, at least 500 adenine nucleotides or more. In one embodiment, the poly(A) tail comprises between 1 and 150 adenine nucleotides. In another embodiment, the poly(A) tail comprises between 90 and 120 adenine nucleotides. In some such embodiments, the poly(A) tail comprises one or more modified nucleosides.

It is contemplated that one or more modifications to the synthetic, modified-RNAs described herein permit greater stability of the synthetic, modified-RNA in a cell or tissue in vivo. To the extent that such modifications permit translation and either reduce or do not exacerbate a cell's innate immune or interferon response to the synthetic, modified-RNA with the modification, such modifications are specifically contemplated for use herein. Generally, the greater the stability of a synthetic, modified-RNA, the more protein can be produced from that synthetic, modified-RNA. Typically, the presence of AU-rich regions in mammalian mRNAs tend to destabilize transcripts, as cellular proteins are recruited to AU-rich regions to stimulate removal of the poly(A) tail of the transcript. Loss of a poly(A) tail of a synthetic, modified-RNA can result in increased RNA degradation. Thus, in one embodiment, a synthetic, modified-RNA as described herein does not comprise an AU-rich region. In particular, it is preferred that the 3′ UTR substantially lacks AUUUA sequence elements.

### Synthesis of Synthetic, Modified RNAs

A modified RNA encoding a YAP protein is synthesized using and/or modified by methods well established in the art, such as those described in “Current Protocols in Nucleic Acid Chemistry,” Beaucage, S. L. et al. (Edrs.), John Wiley & Sons, Inc., New York, N.Y., USA, which is hereby incorporated herein by reference in its entirety. Transcription methods are described further herein in the Examples.

In one embodiment of the aspects described herein, a template for a synthetic, modified-RNA is synthesized using “splint-mediated ligation,” which allows for the rapid synthesis of DNA constructs by controlled concatenation of long oligos and/or dsDNA PCR products and without the need to introduce restriction sites at the joining regions. It can be used to add generic untranslated regions (UTRs) to the coding sequences of genes during T7 template generation. Splint mediated ligation can also be used to add nuclear localization sequences to an open reading frame, and to make dominant-negative constructs with point mutations starting from a wild-type open reading frame. Briefly, single-stranded and/or denatured dsDNA components are annealed to splint oligos which bring the desired ends into conjunction, the ends are ligated by a thermostable DNA ligase and the desired constructs amplified by PCR. A synthetic, modified-RNA is then synthesized from the template using an RNA polymerase in vitro. After synthesis of a synthetic, modified-RNA is complete, the DNA template is removed from the transcription reaction prior to use with the methods described herein.

In some embodiments of these aspects, the synthetic, modified RNAs are further treated with an alkaline phosphatase.

### Oligonucleotide Synthesis and Purification

In some embodiments, the synthetic, modified-RNA can be chemically synthesized using methods described herein. RNA may be produced enzymatically or by partial/total organic synthesis, and modified ribonucleotides can be introduced by in vitro enzymatic or organic synthesis. In one embodiment, each strand is prepared chemically. Methods of synthesizing RNA molecules are known in the art, in particular, the chemical synthesis methods as described in Verma and Eckstein (1998) or as described herein. Generally, synthetic, modified-RNA molecules can by synthesized using solid phase oligonucleotide synthesis methods as described in, for example, Usman et al., U.S. Pat. Nos. 5,804,683; 5,831,071; 5,998,203; 6,117,657; 6,353,098; 6,362,323; 6,437,117; 6,469,158; Scaringe et al., U.S. Pat. Nos. 6,111,086; 6,008,400; 6,111,086.

In a non-limiting example, synthetic, modified-RNA molecules comprising one or more modified nucleotides are synthesized, using solid phase phosphoramidite chemistry, deprotected and desalted on NAP-5 columns (Amersham Pharmacia Biotech, Piscataway, N.J.) using standard techniques (Damha and Olgivie, 1993; Wincott et al., 1995). The oligomers may be purified using ion-exchange high performance liquid chromatography (IE-HPLC), for example, on an Amersham Source 15Q column (1.0 cm.times.25 cm) (Amersham Pharmacia Biotech, Piscataway, N.J.) using a step-linear gradient. Samples are monitored at 260 nm and peaks corresponding to the full-length oligonucleotide species are collected, pooled, desalted, and lyophilized. The purity of each synthesized oligonucleotide is determined by capillary electrophoresis (CE) on a Beckman PACE 5000 (Beckman Coulter, Inc., Fullerton, Calif.). Relative molecular masses of oligomers can be obtained, often within 0.2% of expected molecular mass.

### Plurality of Synthetic, Modified RNAs

One or a combination of modified RNAs encoding YAP proteins may be used. In some embodiments of the aspects described herein, a plurality of different synthetic, modified-RNAs are contacted with, or introduced to, a target tissue in vivo, e.g., a muscle tissue or heart tissue and permit expression of at least two polypeptide products in the desired target tissue. In some embodiments, synthetic, modified-RNA compositions as disclosed herein can comprise two or more synthetic, modified-RNAs, e.g., 2, 3, 4, 5, 6, 7, 8, 9, 10 or more synthetic, modified-RNAs. In some embodiments, the two or more synthetic, modified-RNAs are capable of increasing expression of a desired polypeptide product (e.g., a transcription factor, a cell surface marker, a death receptor, etc.). In some embodiments, where the composition is used for the treatment of a cardiovascular disease or disorder, the composition comprises a MOD-RNA encoding YAP and at least one other MOD-RNA encoding a different cardiac enhancing protein.

In some embodiments, when a plurality of different synthetic, modified-RNAs, synthetic, modified-RNA compositions, or media comprising a plurality of different synthetic, modified-RNAs are used to modulate expression of a desired set of polypeptides, the plurality of synthetic, modified-RNAs can be contacted with, or introduced to, a target tissue in vivo, either simultaneously or subsequently. In other embodiments, the plurality of synthetic, modified-RNAs can be contacted with, or introduced to, a target tissue in vivo separately. In addition, each synthetic, modified-RNA can be administered to the target tissue in vivo according to its own dosage regime. For example, in one embodiment, a composition can be prepared comprising a plurality of synthetic, modified-RNAs, in differing relative amounts or in equal amounts, that is contacted with a target tissue in vivo, such that the plurality of synthetic, modified-RNAs are administered simultaneously. Alternatively, one synthetic, modified-RNA at a time can be administered to a target tissue in vivo (e.g., sequentially). In this manner, the expression desired for each target polypeptide in vivo can be easily tailored by altering the frequency of administration and/or the amount of a particular synthetic, modified-RNA administered. Contacting a target tissue in vivo with each synthetic, modified-RNA separately can also prevent interactions between the synthetic, modified-RNAs that can reduce efficiency of in vivo protein expression. For ease of use and to prevent potential contamination, it is preferred to administer to or contact a target tissue in vivo with a cocktail of different synthetic, modified-RNAs, thereby reducing the number of doses required and minimizing the chance of introducing a contaminant to a target tissue in vivo.

The methods and compositions described herein permit the in vivo protein expression of one or more polypeptides to be tuned to a desired level by varying the amount of each synthetic, modified-RNA transfected. One of skill in the art can easily monitor level of in vivo protein expression encoded by a synthetic, modified-RNA using e.g., Western blotting techniques or immunocytochemistry techniques. A synthetic, modified-RNA can be administered at a frequency and dose to a target tissue in vivo that permits a desired level of in vivo protein expression. As disclosed herein in the Examples, the amount of MOD-RNA administered in vivo determines the amount of in vivo protein expression, and therefore the amount of protein expressed can be controlled based on the amount of MOD-RNA administered to the target tissue in vivo. Accordingly, each different synthetic, modified-RNA can be administered at its own dose and frequency to permit appropriate expression in the target tissue in vivo. In addition, since the synthetic, modified-RNAs administered to a target tissue in vivo is transient in nature (i.e., are degraded over time) one of skill in the art can easily remove or stop the in vivo protein expression from a synthetic, modified-RNA by halting further transfections and permitting the tissue to degrade the synthetic, modified-RNA over time. The synthetic, modified-RNAs will degrade in a manner similar to cellular mRNAs.

Introducing a Synthetic, Modified RNA into a Cell

A synthetic, modified-RNA can be introduced into a target tissue in vivo, e.g., a heart tissue, e.g., for delivery to a cardiomyocyte, in any manner that achieves intracellular delivery of the synthetic, modified-RNA, such that in vivo expression of the polypeptide encoded by the synthetic, modified RNA can occur. As used herein, the term “transfecting a cell” refers to the process of introducing nucleic acids into a cell of a tissue using means for facilitating or effecting uptake or absorption into the tissue, as is understood by those skilled in the art. As the term is used herein, “transfection” does not encompass vector-mediated gene delivery, e.g., viral- or viral particle based delivery methods. Absorption or uptake of a synthetic, modified RNA into a tissue in vivo can occur through unaided diffusive or active cellular processes, or by auxiliary agents or devices. Further approaches are described herein below or known in the art.

In some embodiments, a synthetic, modified RNA can be introduced into a target tissue, e.g., muscle or heart, e.g., a myogenic cell, for example, by transfection, nucleofection, lipofection, electroporation (see, e.g., Wong and Neumann, Biochem. Biophys. Res. Commun. 107:584-87 (1982)), microinjection (e.g., by direct injection of a synthetic, modified RNA), biolistics, cell fusion, and the like.

In one embodiment, a MOD-RNA is introduced into a tissue in vivo, e.g., heart and muscle tissue using a Mega Tran 1.0 transfection reagent (OriGene Technologies Inc.). Alternatively, herein MOD-RNA is introduced into a tissue in vivo, e.g., heart and muscle tissue using lipofectamine (RNAi MAX). While one can optimize the concentration of MOD-RNA delivered, in one embodiment 25 microgram/microliter concentration is used for delivery of about 100 microgram MOD-RNA to a tissue in vivo.

In an alternative embodiment, a synthetic, modified RNA can be delivered using a drug delivery system such as a nanoparticle, a dendrimer, a hydrogel, a biopolymer, a polymer, a liposome, or a cationic delivery system. Positively charged cationic delivery systems facilitate binding of a synthetic, modified RNA (negatively charged polynucleotides) and also enhances interactions at the negatively charged cell membrane to permit efficient cellular uptake. Cationic lipids, dendrimers, or polymers can either be bound to modified RNAs, or induced to form a vesicle or micelle (see e.g., Kim S H., et al (2008) Journal of Controlled Release 129(2):107-116) that encases the modified RNA. Methods for making and using cationic-modified RNA complexes are well within the abilities of those skilled in the art (see e.g., Sorensen, DR., et al (2003) J. Mol. Biol. 327:761-766; Verma, UN., et al (2003) Clin. Cancer Res. 9:1291-1300; Arnold, A Set al (2007) J. Hypertens. 25:197-205, which are incorporated herein by reference in their entirety).

In some embodiments the MOD-RNA described herein can be included in biodegradable polymeric hydrogels, such as those disclosed in U.S. Pat. No. 5,410,016 to Hubbell et al. These polymeric hydrogels can be delivered to a subject and the active compounds released over time as the polymer degrades. Commercially available hydrogels can be supplied either as a dry powder or a partially hydrated paste intended for administration after dispersion in an appropriate amount of aqueous vehicle. These powders are formed by mechanical disruption of cross-linked matrices, such as absorbable gelatin sponges, U.S.P. (e.g., Gelfoam®, Pfizer, Inc. or Surgifoam™, Ethicon, Inc.), or the cakes that are formed during typical chemical or dehydrothermal cross-linking treatment (see, e.g., U.S. Pat. No. 6,063,061; U.S. Patent application pub. No. 2003/0064109). These hydrogels can be based on gelatin, collagen, dextran, chitosan. Other compositions are also used, for example, alginate (U.S. Pat. No. 5,294,446) and synthetic polymers such as polyphosphazines, polyacrylates, polyanhydrides, and polyorthoesters, as well as “block copolymers” such as mixtures of polyethylene oxide and polypropylene glycol (U.S. Pat. Nos. 5,041,138; 5,709,854; 5,736,372). In addition, U.S. Pat. Nos. 5,749,874 and 5,769,899 (both Schwartz et al, 1998) disclose two-component implants, where one component is an anchoring device, made of a relatively hard yet biodegradable material (such as polyglycolic acid, polylactic acid, or combinations thereof), which helps secure and anchor the hydrogel implants and a second component that comprises a more porous and flexible matrix.

Hydrogels can be administered dry, partially hydrated, or fully hydrated. In the fully hydrated state, the hydrogel cannot absorb further fluid, and is fully swollen in size. In contrast, a dry or partially hydrated hydrogel composition has excess adsorptive capacity. Upon administration, dry or partially hydrated hydrogel will absorb fluid leading to a swelling of the gelatin matrix in vivo. Swelling of dry or partially hydrated hydrogel should be considered in the context of administration. If desirable, the polymeric hydrogels can have microparticles or liposomes which include the active compound dispersed therein, providing another mechanism for the controlled release of MOD-RNA described herein, or a nucleic acid encoding the peptide.

In some embodiments of the aspects described herein, the composition further comprises a reagent that facilitates uptake of a synthetic, modified RNA into a cell (transfection reagent), such as an emulsion, a liposome, a cationic lipid, a non-cationic lipid, an anionic lipid, a charged lipid, a penetration enhancer or alternatively, a modification to the synthetic, modified RNA to attach e.g., a ligand, peptide, lipophilic group, or targeting moiety.

The process for delivery of a synthetic, modified RNA to a cell will necessarily depend upon the specific approach for transfection chosen. One preferred approach is to add the RNA, complexed with a cationic transfection reagent (see below) directly to the cell culture media for the cells.

It is also contemplated herein that a first and second synthetic, modified RNA are administered in a separate and temporally distinct manner. Thus, each of a plurality of synthetic, modified RNAs can be administered at a separate time or at a different frequency interval to achieve the desired expression of a polypeptide. Typically, 100 fg to 100 pg of a synthetic, modified RNA is administered per cell using cationic lipid-mediated transfection. Since cationic lipid-mediated transfection is highly inefficient at delivering synthetic, modified RNAs to the cytosol, other techniques can require less RNA. The entire transcriptome of a mammalian cell constitutes about 1 pg of mRNA, and a polypeptide (e.g., a transcription factor) can have a physiological effect at an abundance of less than 1 fg per cell.

### Transfection Reagents

In certain embodiments of the aspects described herein, a synthetic, modified RNA can be introduced into a target tissue in vivo by transfection or lipofection. Suitable agents for transfection or lipofection include, for example but are not limited to, calcium phosphate, DEAE dextran, lipofectin, lipofectamine, DIMRIE C™, Superfect™, and Effectin™ (Qiagen™), Unifectin™, Maxifectin™, DOTMA, DOGS™ (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)). In the Examples disclosed herein, the inventors introduced MOD-RNA into a tissue in vivo, e.g., heart and muscle tissue using a Mega Tran 1.0 transfection reagent (OriGene Technologies Inc.). Alternatively in some embodiments, MOD-RNA was introduced into a tissue in vivo, e.g., heart and muscle tissue, using lipofectamine (RNAi MAX). While one can optimize the concentration of MOD-RNA delivered, in one embodiment, the inventors used 25 μg/μl concentration for delivery of about 100 μg MOD-RNA to a tissue in vivo.

A synthetic, modified RNA can be transfected into a target tissue in vivo as disclosed herein as a complex with cationic lipid carriers (e.g., Oligofectamine™) or non-cationic lipid-based carriers (e.g., Transit-TKOTM™, Minus Bio LLC, Madison, Wis.). Successful introduction of a modified RNA into a target tissue in vivo can be monitored using various known methods. For example, transient transfection of a target tissue in vivo herein can be signaled with a reporter, such as a fluorescent marker, such as Green Fluorescent Protein (GFP), as shown herein, or using luciferase reporter using bioluminescence detection, or using beta-gal reporter from a Cre-recombinase mouse model transfected with MOD-RNA encoding cre recombinase. Successful transfection of a target tissue in vivo with modified RNA can also be determined by measuring the protein expression level of the target polypeptide by e.g., Western Blotting or immunocytochemistry.

In some embodiments of the aspects described herein, the synthetic, modified RNA is introduced into a target tissue in vivo using a transfection reagent. Some exemplary transfection reagents include, for example, cationic lipids, such as lipofectin (Junichi et al, U.S. Pat. No. 5,705,188), cationic glycerol derivatives, and polycationic molecules, such as polylysine (Lollo et al., PCT Application WO 97/30731). Examples of commercially available transfection reagents include, for example Lipofectamine™ (Invitrogen; Carlsbad, Calif.), Lipofectamine 2000™ (Invitrogen; Carlsbad, Calif.), 293Fectin™ (Invitrogen; Carlsbad, Calif.), Cellfectin™ (Invitrogen; Carlsbad, Calif.), DMRIE-C™ (Invitrogen; Carlsbad, Calif.), FreeStyle™ MAX (Invitrogen; Carlsbad, Calif.), Lipofectamine™ 2000 CD (Invitrogen; Carlsbad, Calif.), Lipofectamine™ (Invitrogen; Carlsbad, Calif), RNAiMAX (Invitrogen; Carlsbad, Calif.), Oligofectamine™ (Invitrogen; Carlsbad, Calif.), Optifect™ (Invitrogen; Carlsbad, Calif), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, Wis.), TransFast™ Transfection Reagent (Promega; Madison, Wis.), Tfx™-20 Reagent (Promega; Madison, Wis.), Tfx™-50 Reagent (Promega; Madison, Wis.), DreamFect™ (OZ Biosciences; Marseille, France), EcoTransfect (OZ Biosciences; Marseille, France), TransPass.sup.a D1 Transfection Reagent (New England Biolabs; Ipswich, Mass., USA), LyoVec™/LipoGen™ (Invitrogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif, USA), Cytofectin Transfection Reagent (Genlantis; San Diego, Calif., USA), BaculoPORTER Transfection Reagent (Genlantis; San Diego, Calif., USA), TroganPORTERT™ transfection Reagent (Genlantis; San Diego, Calif, USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif, USA), or HiFect™ (B-Bridge International, Mountain View, Calif., USA), among others.

In other embodiments, highly branched organic compounds, termed “dendrimers,” can be used to bind the exogenous nucleic acid, such as the synthetic, modified RNAs described herein, and introduce it into a target tissue in vivo.

In other embodiments of the aspects described herein, non-chemical methods of transfection are contemplated. Such methods include, but are not limited to, electroporation (methods whereby an instrument is used to create micro-sized holes transiently in the plasma membrane of cells under an electric discharge), sono-poration (transfection via the application of sonic forces to cells), and optical transfection (methods whereby a tiny (.about.1 .mu.m diameter) hole is transiently generated in the plasma membrane of a cell using a highly focused laser). In other embodiments, particle-based methods of transfections are contemplated, such as the use of a gene gun, whereby the nucleic acid is coupled to a nanoparticle of an inert solid (commonly gold) which is then “shot” directly into the target cell's nucleus; “magnetofection,” which refers to a transfection method, that uses magnetic force to deliver exogenous nucleic acids coupled to magnetic nanoparticles into target cells; “impalefection,” which is carried out by impaling cells by elongated nanostructures, such as carbon nanofibers or silicon nanowires which have been coupled to exogenous nucleic acids.

Other agents may be utilized to enhance the penetration of the administered nucleic acids, including glycols, such as ethylene glycol and propylene glycol, pyrrols such as 2-pyrrol, azones, and terpenes, such as limonene and menthone.

### Delivery Formulations and Pharmaceutical Compositions

In some embodiments, a synthetic, modified YAP-encoding RNA molecule is delivered to a target tissue in vivo encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171. 1998); Sakuma S Ret al (Mucoadhesion of polystyrene nanoparticles having surface hydrophilic polymeric chains in the gastrointestinal tract. Int J Pharm 177:161. 1999); Virovic Let al. Novel delivery methods for treatment of viral hepatitis: an update. Expert Opin Drug Deliv 2:707. 2005); and Zimmermann E et al, Electrolyte- and pH-stabilities of aqueous solid lipid nanoparticle (SLN) dispersions in artificial gastrointestinal media. Eur J Pharm Biopharm 52:203. 2001). In some embodiments, where the composition comprises more than one MOD-RNA molecule, each MOD-RNA formulated as its own nanoparticle formulation and the pharmaceutical composition comprises a plurality of MOD-RNA-nanoparticle formulations. In alternative embodiments, a nanoparticle can comprise a plurality of different synthetic modified-RNAs encoding different proteins. Each method represents a separate embodiment of the present invention.

In some embodiment, one or MOD-RNA is delivered to a target tissue in vivo in a vesicle, e.g. a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid). In some embodiments, where the composition comprises more than one MOD-RNA molecule, each MOD-RNA can be formulated as its own liposome formulation, and a pharmaceutical composition can comprise a plurality of MOD-RNA-liposome formulations. In alternative embodiments, a liposome can comprise a plurality of different synthetic modified-RNAs encoding different proteins.

In some embodiments, compositions comprising at least one MOD-RNA for delivery to a target tissue in vivo as disclosed herein can be, in another embodiment, administered to a subject by any method known to a person skilled in the art, such as parenterally, intramuscularly, intra-dermally, subcutaneously, intra-peritonealy, or intra-ventricularly. In another embodiment of methods and compositions of the present invention, compositions comprising at least one MOD-RNA for delivery to a target tissue in vivo as disclosed herein are formulated in a form suitable for injection, i.e. as a liquid preparation. Suitable liquid formulations include solutions, suspensions, dispersions, emulsions, oils and the like. In another embodiment of the present invention, the active ingredient is formulated in a capsule, e.g., a slow release capsule.

In other embodiments, the pharmaceutical compositions comprising at least one MOD-RNA for delivery to a target tissue in vivo as disclosed herein can be administered by intra-arterial, or intramuscular injection of a liquid preparation. Suitable liquid formulations include solutions, suspensions, dispersions, emulsions, oils and the like. In another embodiment, the pharmaceutical compositions comprising at least one MOD-RNA as disclosed herein can be administered intra-arterially and are thus formulated in a form suitable for intra-arterial administration.

In another embodiment, the pharmaceutical compositions for delivery to a target tissue in vivo can administered intramuscularly and are thus formulated in a form suitable for intramuscular administration. Intramuscular injection can be into cardiac muscle, diagram and limb muscles, as disclosed herein.

In another embodiment, a pharmaceutical compositions comprising at least one MOD-RNA for delivery to a target tissue in vivo as disclosed herein can be administered topically to body surfaces and are thus formulated in a form suitable for topical administration. Suitable topical formulations include gels, ointments, creams, lotions, drops and the like. For topical administration, the compositions or their physiologically tolerated derivatives are prepared and applied as solutions, suspensions, or emulsions in a physiologically acceptable diluent with or without a pharmaceutical carrier. In some embodiments, where protein expression in vivo in the eye is beneficial, a pharmaceutical composition comprising at least one MOD-RNA as disclosed herein is formulated in the form of an eye drop.

As used herein “pharmaceutically acceptable carriers or diluents” are well known to those skilled in the art. The carrier or diluent may be may be, in various embodiments, a solid carrier or diluent for solid formulations, a liquid carrier or diluent for liquid formulations, or mixtures thereof. In another embodiment, solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof. In other embodiments, pharmaceutically acceptable carriers for liquid formulations may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In another embodiment, a compositions for delivery of a MOD-RNA to a target tissue in vivo further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention.

In another embodiment, a pharmaceutical composition for delivery of a MOD-RNA to a target tissue in vivo can comprise a MOD-RNA in a controlled-release composition, i.e. a composition in which the compound is released over a period of time after administration. Controlled- or sustained-release compositions include formulation in lipophilic depots (e.g. fatty acids, waxes, oils). In another embodiment, a composition for delivery of a MOD-RNA to a target tissue in vivo is an immediate-release composition, i.e. a composition in which the entire compound is released immediately after administration.

In another embodiment, for delivery of a MOD-RNA to a target tissue in vivo, one can modify a MOD-RNA of the present invention by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications also increase, in another embodiment, the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In another embodiment, a composition for delivery of a MOD-RNA to a target tissue in vivo is formulated to include a neutralized pharmaceutically acceptable salt form. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like. Each of the above additives, excipients, formulations and methods of administration represents a separate embodiment of the present invention.

In some embodiments of the aspects described herein, involving in vivo administration of synthetic, modified-RNAs or compositions thereof to a target tissue in vivo, are formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

A compositions comprising at least one MOD-RNA as disclosed herein can be formulated into any of many possible administration forms, including a sustained release form. The compositions can be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

A composition comprising at least one MOD-RNA as disclosed herein can be prepared and formulated as emulsions for the delivery of synthetic, modified-RNAs. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain further components in addition to the dispersed phases, and the active drug (i.e., synthetic, modified-RNA) which can be present as a solution in either the aqueous phase, oily phase or itself as a separate phase. Pharmaceutical excipients such as emulsifiers, stabilizers, dyes, and anti-oxidants can also be present in emulsions as needed. Emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

As noted above, liposomes can optionally be prepared to contain surface groups to facilitate delivery of liposomes and their contents to specific cell populations. For example, a liposome can comprise a surface groups such as antibodies or antibody fragments, small effector molecules for interacting with cell-surface receptors, antigens, and other like compounds.

Surface groups can be incorporated into the liposome by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

A number of liposomes comprising nucleic acids are known in the art. WO 96/40062 (Thierry et al.) discloses methods for encapsulating high molecular weight nucleic acids in liposomes. U.S. Pat. No. 5,264,221 (Tagawa et al.) discloses protein-bonded liposomes and asserts that the contents of such liposomes can include an RNA molecule. U.S. Pat. No. 5,665,710 (Rahman et al.) describes certain methods of encapsulating oligodeoxynucleotides in liposomes. WO 97/04787 (Love et al.) discloses liposomes comprising RNAi molecules targeted to the raf gene. In addition, methods for preparing a liposome composition comprising a nucleic acid can be found in e.g., U.S. Pat. Nos. 6,011,020; 6,074,667; 6,110,490; 6,147,204; 6, 271, 206; 6,312,956; 6,465,188; 6,506,564; 6,750,016; and 7,112,337. Each of these approaches can provide delivery of a synthetic, modified-RNA as described herein to a cell.

In some embodiments of the aspects described herein, a composition comprising at least one MOD-RNA for in vivo protein expression in a target tissue as disclosed herein can be encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171.1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al,

Electrolyte- and pH-stabilities of aqueous solid lipid nanoparticle (SLN) dispersions in artificial gastrointestinal media. Eur J Pharm Biopharm 52:203. 2001), the contents of which are herein incorporated in their entireties by reference.

### Ligands to Enhance YAP MOD-RNA Uptake

If desired, uptake of a modified RNA encoding a YAP protein is enhanced via a ligand. In one embodiment, a ligand alters the cellular uptake, intracellular targeting or half-life of a synthetic, modified-RNA into which it is incorporated. In some embodiments a ligand provides an enhanced affinity for a selected target tissue, e.g., tissue or cell type, or intracellular compartment, e.g., mitochondria, cytoplasm, peroxisome, lysosome, as, e.g., compared to a composition absent such a ligand. Preferred ligands do not interfere with expression of a polypeptide from the synthetic, modified-RNA.

Ligands can include, for example, a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid. Examples of polyamino acids include polylysine (PLL), poly L aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, (e.g., a lectin, glycoprotein, lipid or protein), or an antibody, that binds to a specified cell type such as a fibroblast cell. In some embodiments, where the target cell is an endothelial cell, a endothelial cell targeting agents is a vWF protein or fragment thereof. In some embodiments, other targeting group useful in the methods as disclosed herein can be, for example, a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B 12, biotin, or an RGD peptide or RGD peptide mimetic, among others.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, O3-(oleoyl)cholenic acid, dimethoxytrityl, or phenoxazine) and peptide conjugates (e.g., antennapedia peptide, Tat peptide), alkylating agents, amino, mercapto, PEG (e.g., PEG-40K), MPEG, [MPEG]2, polyamino, alkyl, substituted alkyl, radiolabeled markers, enzymes, haptens (e.g. biotin), and transport/absorption facilitators (e.g., aspirin, vitamin E, folic acid).

Ligands can be proteins, e.g., glycoproteins, or peptides, e.g., molecules having a specific affinity for a co-ligand, or antibodies e.g., an antibody, that binds to a specified cell type such as a fibroblast cell, or other cell useful in the production of polypeptides. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose.

The ligand can be a substance, e.g., a drug, which can increase the uptake of the synthetic, modified-RNA or a composition thereof into the cell, for example, by disrupting the cell's cytoskeleton, e.g., by disrupting the cell's microtubules, microfilaments, and/or intermediate filaments. The drug can be, for example, taxol, vincristine, vinblastine, cytochalasin, nocodazole, japlakinolide, latrunculin A, phalloidin, swinholide A, indanocine, or myoservin.

One exemplary ligand is a lipid or lipid-based molecule. A lipid or lipid-based ligand can (a) increase resistance to degradation, and/or (b) increase targeting or transport into a target cell or cell membrane. A lipid based ligand can be used to modulate, e.g., binding of the modified RNA composition to a target cell.

In another aspect, the ligand is a moiety, e.g., a vitamin, which is taken up by a host cell. Exemplary vitamins include vitamin A, E, and K. Other exemplary vitamins include B vitamin, e.g., folic acid, B 12, riboflavin, biotin, pyridoxal or other vitamins or nutrients taken up, for example, by cancer cells. Also included are HSA and low density lipoprotein (LDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

In some embodiments the term a “cell permeation peptide” is capable of permeating a cell, e.g., a mammalian cell, such as a human cell, as well, as a peptide which permeates the blood-brain barrier. Cell permeation peptides are well known in the art, and include, but are not limited to, for example, an α-helical linear peptide (e.g., LL-37 or Ceropin P1), a disulfide bond-containing peptide (e.g., α-defensin, (β-defensin or bactenecin), or a peptide containing only one or two dominating amino acids (e.g., PR-39 or indolicidin), and bipartite amphipathic peptides, such as MPG, which is derived from the fusion peptide domain of HIV-1 gp41 and the NLS of SV40 large T antigen (Simeoni et al., Nucl. Acids Res. 31:2717-2724, 2003).

### Methods for Further Avoiding a Cell's Innate Immune or Interferon Response

In some embodiments, a composition comprising at least one MOD-RNA as disclosed herein also comprise an agent to reduce the innate immune mediated response. In one embodiment, the composition further comprises a modified RNA encoding an interferon scavenging agent (e.g., a soluble interferon receptor) to further reduce the innate immune response of tissue.

In some embodiments, small molecules that inhibit the innate immune response in cells, such as chloroquine (a TLR signaling inhibitor) and 2-aminopurine (a PKR inhibitor), can also be administered in combination with the composition comprising at least one MOD-RNA for in vivo protein expression as disclosed herein. Some non-limiting examples of commercially available TLR-signaling inhibitors include BX795, chloroquine, CLI-095, OxPAPC, polymyxin B, and rapamycin (all available for purchase from INVIVOGEN™). In addition, inhibitors of pattern recognition receptors (PRR) (which are involved in innate immunity signaling) such as 2-aminopurine, BX795, chloroquine, and H-89, can also be used in the compositions and methods comprising at least one MOD-RNA for in vivo protein expression as disclosed herein. Additionally, the compositions comprising at least one MOD-RNA for in vivo protein expression as disclosed herein can further comprise cell-penetrating peptides that inhibit proteins in the immunity pathways. Some non-limiting examples of commercially available cell-penetrating peptides include Pepin-MYD (INVIVOGEN™) or Pepinh-TRIF (INVIVOGEN™). An oligodeoxynucleotide antagonist for the Toll-like receptor signaling pathway can also be added to a comprising at least one MOD-RNA for in vivo protein expression as disclosed herein to reduce immunity signaling.

Another method for reducing the immune response of a tissue transfected with the synthetic, modified RNAs described herein is to co-transfect MOD-RNAs that encode negative regulators of innate immunity such as NLRX1. Accordingly, in some embodiments, a composition comprising at least one MOD-RNA for in vivo protein expression as disclosed herein comprises a MOD-RNA encoding one or more, or any combination of NLRX1, NS1, NS3/4A, or A46R. Additionally, in some embodiments, a composition comprising at least one MOD-RNA as disclosed herein can also comprise a synthetic, modified-RNA encoding inhibitors of the innate immune system to avoid the innate immune response generated by the tissue or the subject.

It is also contemplated herein that, in some embodiments, in a research setting one of skill in the art can avoid the innate immune response generated in the cell by using cells genetically deficient in antiviral pathways (e.g., VISA knockout cells).

In another embodiment, a composition comprising at least one MOD-RNA as disclosed herein can further comprise an immunosuppressive agent. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 20020182211. In one embodiment, a immunosuppressive agent is cyclosporine A. Other examples include myophenylate mofetil, rapamicin, and anti-thymocyte globulin. In one embodiment, the immunosuppressive drug is administered in a composition comprising at least one MOD-RNA as disclosed herein, or can be administered in a separate composition but simultaneously with, or before or after administration of a composition comprising at least one MOD-RNA to the subject. An immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the MOD-RNA as disclosed herein.

### Assaying YAP Efficacy

The efficacy of a MOD-RNA encoding a YAP protein is determined in an animal model by assessing the degree of cardiac recuperation that ensues from treatment with the MOD-RNA. A number of animal models are available for such testing. For example, hearts can be cryoinjured by placing a precooled aluminum rod in contact with the surface of the anterior left ventricle wall (Murry et al., J. Clin. Invest. 98:2209, 1996; Reinecke et al., Circulation 100:193, 1999; U.S. Pat. No. 6,099,832). In larger animals, cryoinjury can be inflicted by placing a 30-50 mm copper disk probe cooled in liquid N2 on the anterior wall of the left ventricle for approximately 20 min (Chiu et al., Ann. Thorac. Surg. 60:12, 1995). Infarction can be induced by permanently ligating the left main or left anterior descending coronary artery (Li et al., J. Clin. Invest. 100:1991, 1997). Ischemia repurfusion injury can also be modeled by transient left main or left anterior descending coronary artery occlusion. Injured sites are treated with cell preparations of this invention, and the heart tissue is examined by histology for the presence of the cells in the damaged area. Cardiac function can be monitored by determining such parameters as left ventricular end-diastolic pressure, developed pressure, rate of pressure rise, and rate of pressure decay.

In some embodiments, a MOD-RNA encoding a YAP protein may be administered in any physiologically acceptable excipients. A composition comprising a MOD-RNA encoding a protein of interest can be delivered to a target tissue, e.g., a heart by injection, catheter, or the like.

In some embodiments, a composition comprising a MOD-RNA encoding a protein of interest for in vivo protein expression in target tissue as disclosed herein can be supplied in the form of a pharmaceutical composition, comprising an isotonic excipient prepared under sufficiently sterile conditions for human administration. Choice of the cellular excipient and any accompanying elements of the composition will be adapted in accordance with the route and device used for administration. A composition comprising a MOD-RNA encoding a protein of interest for in vivo protein expression in target tissue as disclosed herein can also comprise or be accompanied with one or more other ingredients that facilitate the therapeutic effect, and include use of scaffolds, as well as addition of other cells. Suitable ingredients include complementary cell types, especially endothelial cells. In another embodiment, the composition may comprise resorbable or biodegradable matrix scaffolds.

Another aspect of the present invention relates to the administration of a composition comprising a MOD-RNA encoding a protein of interest for in vivo protein expression in target tissue as disclosed herein either systemically or to a target anatomical site. The composition comprising a MOD-RNA encoding a protein of interest for in vivo protein expression in target tissue as disclosed herein can be introduced into or nearby a subject's target tissue, e.g., a heart, for example, or may be administered systemically, such as, but not limited to, intra-arterial or intravenous administration. In alternative embodiments, a composition comprising a MOD-RNA encoding a protein of interest for in vivo protein expression in target tissue as disclosed herein can be administered in various ways as would be appropriate to deliver to a subject's cardiovascular system, including but not limited to parenteral, including intravenous and intraarterial administration, intrathecal administration, intraventricular administration, intraparenchymal, intracranial, intracisternal, intrastriatal, and intranigral administration. Optionally, a composition comprising a MOD-RNA encoding a protein of interest for in vivo protein expression in target tissue as disclosed herein are administered in conjunction with an immunosuppressive agent.

A composition comprising a MOD-RNA encoding a protein of interest for in vivo protein expression in target tissue as disclosed herein s disclosed herein can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is defined in the definitions sections and is determined by such considerations as are known in the art. The amount must be effective to halt the disease progression and/or to achieve improvement, including but not limited to improved survival rate or more rapid recovery, or improvement or elimination of symptoms and other indicators as are selected as appropriate measures by those skilled in the art. A composition comprising a MOD-RNA encoding a protein of interest for in vivo protein expression in target tissue as disclosed herein can be administered to a subject can take place but is not limited to the following locations: clinic, clinical office, emergency department, hospital ward, intensive care unit, operating room, catheterization suites, and radiologic suites.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### The Major Cardiac Interaction Partner of TEAD1 Changes with Postnatal Age

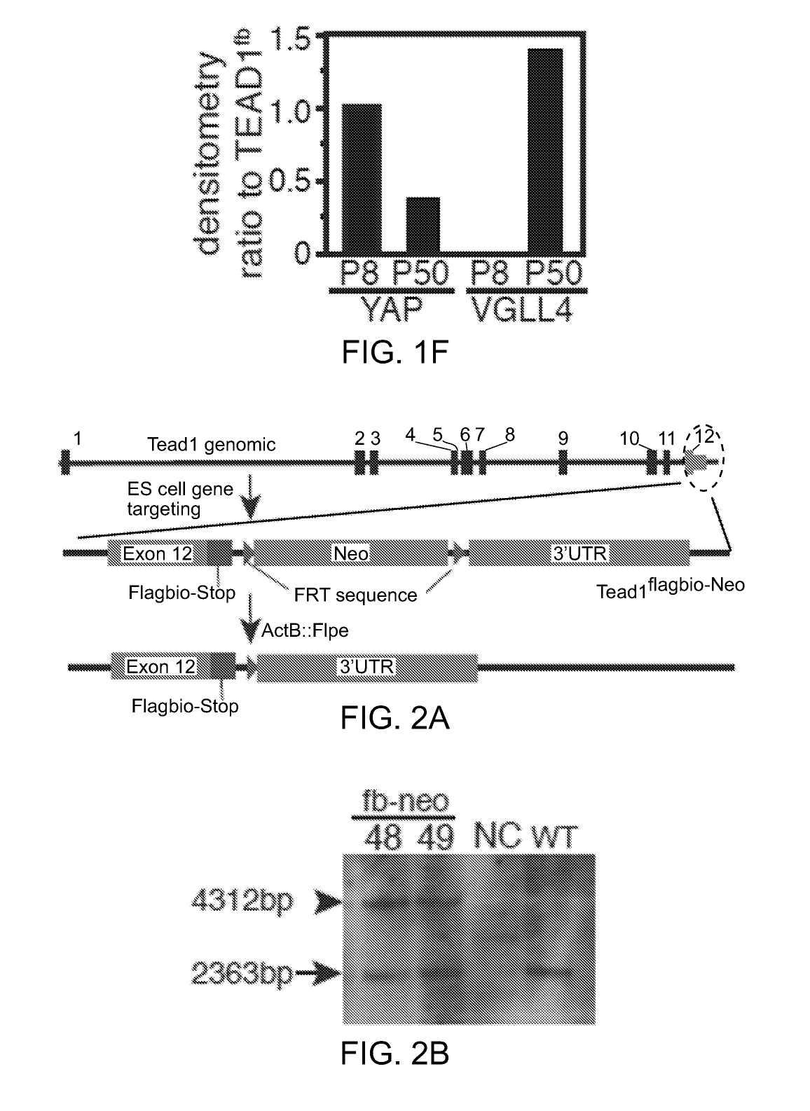

In this example, the protein expression of YAP, TEAD1, and VGLL4 was studied in several adult tissues to understand their function in regulating the growth of various organs. During heart development, the Hippo-YAP pathway regulates cardiomyocyte (CM) proliferation (Heallen et al., 2011; von Gise et al., 2012; Xin et al., 2011). YAP and TEAD1 are terminal effectors of the Hippo-YAP pathway. VGLL4, a TEAD1 binding protein that was found to modulate the potency of overexpressed YAP in the liver (Koontz et al., 2013), was previously reported to have cardiac-restricted RNA expression (Chen et al., 2004). YAP was widely expressed in adult mouse tissues, as demonstrated previously (von Gise et al., 2012). Robust VGLL4 expression was detected in the heart, with lower levels were also present in the brain, liver and lung (FIG. 1A). TEAD1 protein was abundant in the lung, less expressed in the heart, and undetectable in the other organs examined (FIG. 1A). The expression of these proteins in heart was also measured at several different ages. Interestingly, VGLL4 expression increased from low levels in the newborn heart to high levels in the adult heart (FIG. 1B). TEAD1 and YAP levels were anti-correlated with VGLL4 and decreased with age (FIG. 1B). To determine whether these proteins were expressed in adult CMs or non-CMs, hearts were dissociated and cell populations were isolated and purified. VGLL4 and TEAD1 were mainly expressed in CMs rather than non-CMs, whereas YAP was predominantly expressed in non-CMs (FIG. 1C).

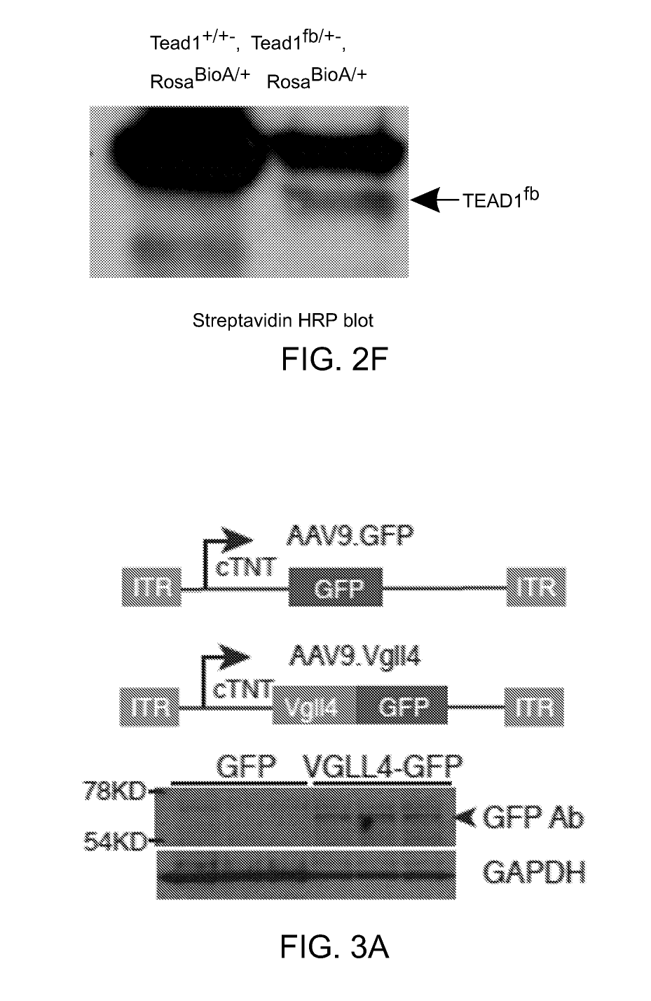

The observed changes in expression indicated TEAD1′s primary interaction partner changed from YAP to VGLL4 between newborn and adult heart. To further test the identity of TEAD1′s interaction partners during development, a Tead1 knock-in allele, Tead1^{fb}, was generated that contains the FLAG and AviTag (Bio) (He et al., 2012) epitope tags fused to the Tead1 C-terminus (Tead1^{fb}; FIGS. 2A-2B). The E. coli enzyme BirA specifically recognizes and biotinylates the Bio tag (de Boer et al., 2003), permitting high affinity capture (e.g., “pull-down”) on immobilized streptavidin (SA). Tead1^{fb/fb}; Rosa26^{BirA/BirA} mice survived normally to weaning, had no overt phenotype (FIGS. 2C-2D), and expressed biotinylated TEAD1^{fb} (FIGS. 2E-2F). TEAD1^{fb} was precipitated with its interacting proteins on SA beads, from heart samples at postnatal day 1 (P1), P8, and adult (P50) (FIGS. 1D-1F). This experiment showed that TEAD1 and VGLL4 strongly interacted in the adult heart, but not the neonatal (P1 or P8) heart (FIGS. 1D-1F). TEAD1 and YAP interaction showed the opposite pattern, with a strong interaction detected in the neonatal heart and a weaker interaction in the adult heart (FIGS. 1E-1F).

### Example 2

### Precocious VGLL4 Overexpression did not Suppress Neonatal Cardiac Growth

To test whether VGLL4 limits cardiomyocyte (CM) proliferation by reducing TEAD1-YAP interaction, VGLL4 was overexpressed in the newborn heart using adeno-associated virus serotype 9 (AAV9), an efficient cardiac gene delivery vector (Lin and Pu, 2014). Constructs were generated for AAV9.VGLL4-GFP (AAV9.VGLL4) and AAV9.GFP, which express VGLL4-GFP fusion protein or GFP, respectively, from the cardiomyocyte-specific chicken cardiac troponin T (cTNT) promoter (FIG. 3A), and injected into P1 wild-type pups. The hearts were then analyzed seven days later. Immunoblots confirmed cardiac VGLL4-GFP expression (FIG. 3A). Unexpectedly, AAV9.VGLL4 did not significantly change heart function or size compared to untreated (Ctrl) or AAV9.GFP-treated hearts (FIGS. 3B-3D). Staining for phosphohistone H3 (pH3), an M phase cell cycle marker, indicated that CM cell cycle activity was not significantly changed by AAV9.VGLL4 compared to AAV-GFP (FIGS. 3E-3F). Consistent with this observation, the expression of cell cycle genes Aurka, Cdc20, and Ccna2 did not differ significantly between groups (FIG. 3G).

Next the effect of AAV9.VGLL4 on YAP-TEAD1 interaction was tested. In adult heart, where there was robust interaction between TEAD1 and endogenous VGLL4, it was confirmed that VGLL4-GFP bound TEAD1 (FIG. 4A). This result indicated that the GFP tag did not disrupt VGLL4-TEAD1 interaction. However, VGLL4-GFP binding to TEAD1 in the neonatal heart was not detected (FIG. 4B). This indicated that the lack of robust interaction between VGLL4 and TEAD1 in neonatal heart was not solely due to lower neonatal VGLL4 expression. and that forced expression of VGLL4-GFP in neonatal heart was not sufficient to drive its interaction with TEAD1. This indicates additional factors may regulate this interaction, which could limit the interaction of overexpressed VGLL4-GFP and TEAD1 thereby accounting for the lack of phenotype in AAV9.VGLL4-treated heart.

### Example 3

### VGLL4 Activity is Regulated by Acetylation of its Tondu (TDU) Domain

Factors that govern VGLL4-TEAD1 interaction in the neonatal heart were characterized. Post-translational modification was studied as one potential regulatory mechanism. Adopting a candidate strategy, VGLL4 acetylation was first investigated.

Histone acetyltransferases such as p300 or CREB-binding protein (CREBBP) acetylate lysine resides of non-histone proteins, including transcription factors, in addition to histones (Chan and La Thangue, 2001). In co-immunoprecipitation assays, VGLL4 robustly interacted with p300, whereas its interaction with CBP was considerably weaker (FIG. 5A). Moreover, p300 but not CBP heavily acetylated VGLL4 (FIG. 5A). To identify VGLL4 acetylation sites, the VGLL4-GFP fusion protein and p300 was co-expressed in HEK293T cells. Immunoprecipitated VGLL4-GFP was then analyzed by mass spectrometry. Several acetylation sites were identified, with the highest fraction of acetylated residues occurring at lysine 225 (K255; FIG. 5B). To confirm this observation, a mutation was introduced in VGLL4 at K225 to substitute an arginine (VGLL4[R]), which is structurally similar to lysine but cannot be acetylated. In co-transfected cells, VGLL4[R] was less heavily acetylated by p300 than the wild-type protein (FIG. 5C, see ratio of KAc to total VGLL4 in lane 5 vs. 3). This is consistent with VGLL4 being acetylated predominantly, but not solely at K225. Vestigial-like family members interact with TEAD through their Tondu (TDU) domains (Koontz et al., 2013), and VGLL4 contains two TDU domains. K225 is located in the first TDU domain of human VGLL4 and is conserved among vertebrate VGLL4 proteins but not in TDU domains from other proteins (FIG. 5D).

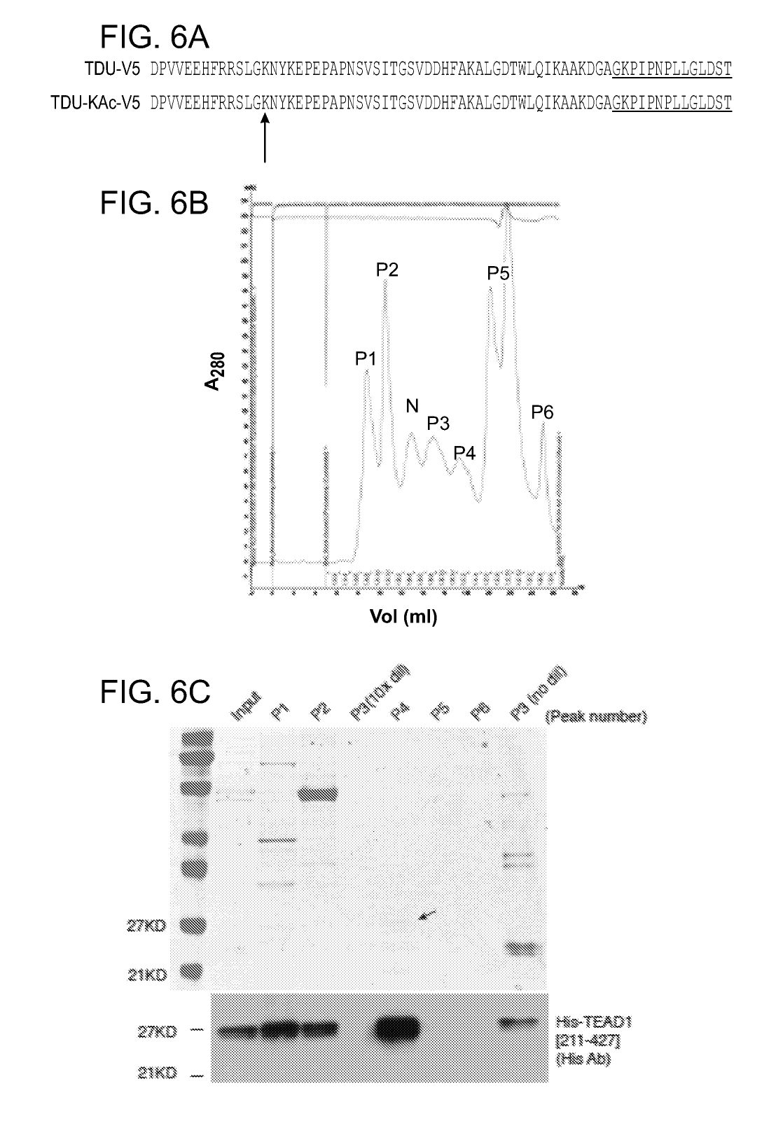

The location of K225 within the TEAD1 binding domain of VGLL4 indicated that acetylation of K225 modulates VGLL4-TEAD interaction. To study the interaction of VGLL4 and TEAD, peptides corresponding to the VGLL4 TDU domains were synthesized, with or without K225 acetylation (FIG. 6A). These V5 epitope-tagged VGLL4 peptides were co-incubated with recombinant, His-tagged TEAD1 (residues 211-427), which contains the YAP and VGLL4 interaction domains (Jiao et al., 2014) (FIGS. 6B-6C). The interaction between VGLL4 peptide and His-TEAD1 [211-427] was measured by immunoprecipitation followed by western blotting (FIG. 5E). The synthetic non-acetylated VGLL4-TDU domain peptide bound TEAD1 in a dose-dependent manner. In contrast, the interaction between the acetylated VGLL4-TDU peptide and TEAD1 could not be detected reproducibly. To quantify this result, a recently developed nano-scale photonic interaction assay (Yang et al., 2014). His-TEAD1 [211-427] was immobilized on a nanobeam sensor and then incubated with increasing concentrations of the VGLL4-TDU peptide. The non-acetylated VGLL4-TDU peptide induced a concentration-dependent resonance shift of the sensor (FIG. 5F), indicative of binding to TEAD1. Fitting the curve to the Langmuir equation yielded a VGLL4-TEAD1 interaction affinity of 3.1±1.3 nM. In contrast, the acetylated VGLL4-TDU peptide did not induce a resonance shift up to a peptide concentration of 1 μg/ml (FIG. 5F). Together these results indicated that VGLL4 acetylation at K225 strongly impeded its binding to TEAD1.

To study the effect of VGLL4 acetylation on VGLL4-TEAD1 interaction in a cellular context, VGLL4-GFP or VGLL4[R]-GFP was co-expressed with Tead1^{tfb} in 293T cells and their interaction was analyzed by co-immunoprecipitation. TEAD1^{fb} pulled down more VGLL4[R] than VGLL4 (FIG. 3G), indicating that acetylation at K225 reduced VGLL4-TEAD1 interaction in vivo. Consistent with this result, VGLL4[R] inhibited TEAD1-YAP interaction with greater potency than wild-type VGLL4 (FIG. 5G). The functional effect of VGLL4 and its acetylation on TEAD1-YAP transcriptional activity was measured using 8xGTIIC-Luci (Dupont et al., 2011), a luciferase reporter driven by a multimerized TEAD1 binding site. TEAD1 alone weakly stimulated reporter activity, and this was inhibited by both VGLL4 and VGLL4[R] (FIG. 3H). Consistent with its broad role as a transcriptional co-activator, p300 strongly stimulated TEAD1 transcriptional activity. VGLL4 partially blocked this stimulation, as expected based on its antagonism of TEAD1-YAP interaction (FIG. 5G). Compared to VGLL4, VGLL4[R] more potently blocked p300 stimulation (FIG. 5H), in agreement with the more potent disruption of TEAD1-YAP interaction by VGLL4[R] (FIG. 5G).

To determine if VGLL4 acetylation affects VGLL4 and TEAD1 interaction in cardiomyocytes (CMs), the proximity ligation assay (PLA) (Soderberg et al., 2006) was used to study the in situ interaction between VGLL4 and TEAD1 in cultured neonatal rat ventricular cardiomyocytes (NRVMs), with or without the overexpression of p300. NRVMs stained with TEAD1 or VGLL4 antibodies individually showed that TEAD1 was localized in the nucleus, while VGLL4 was located in both cytoplasm and nucleus (FIG. 6D). The PLA assay showed in situ TEAD1-VGLL4 interaction primarily in the nucleus (FIG. 5I). The overexpression of p300 significantly reduced TEAD1-VGLL4 interaction (P<0.05; FIGS. 5I-5J). These data indicated that acetylation of VGLL4 decreased its interaction with TEAD1 in CMs.

To determine whether VGLL4 is acetylated endogenously in CMs in vivo, an antibody was generated that recognized VGLL4 acetylated at K225 and that did not bind VGLL4 lacking this modification (FIGS. 6E-6F). The antibody was used to assess acetylation of the corresponding residue of VGLL4 in murine heart (murine K216 corresponds to K225 of human VGLL4). Immunoblotting of mouse hearts showed that the level of mVGLL4-K216Ac and total VGLL4 increased 3 and 6 times, respectively, between P6 and P60 (FIG. 5K). This indicated that the ratio of mVGLL4-K216Ac to total VGLL4 decreased with age. This result is consistent with the age related decrease in the fraction of acetylated VGLL4 and p300 expression in the mouse heart (FIG. 6G).

To assess the potential relevance of developmental changes in VGLL4 and VGLL4 acetylation to the human heart, their expression as well as the expression of TEAD1, YAP, and p300 was examined in normal human myocardium at different postnatal ages (FIG. 5L). TEAD1 protein levels declined with age, as was observed in mouse. Unlike mouse, YAP and VGLL4 expression were relatively constant in 2-18 year-old hearts. However, VGLL4-K225Ac levels strongly decreased with postnatal age, as did expression of p300, paralleling the observations in the mouse. These results indicated that developmentally regulated protein expression and VGLL4-K225 acetylation contribute to regulation of YAP-TEAD activity in the human heart. Whereas VGLL4 expression may contribute to stage-specific regulation of VGLL4-TEAD interaction, VGLL4 acetylation appears to predominate in humans.

In conclusion, the data indicate that acetylation of VGLL4 within its TDU domain at K225 inhibits its binding to TEAD1 in the heart. Mutation of VGLL4 K225 to arginine abrogated this inhibitory acetylation and promoted TEAD1-VGLL4 interaction.

### Example 4

### VGLL4 Suppresses YAP Activity Partially by Promoting TEAD1 Degradation

In studies on the interaction of VGLL4 with TEAD1 in 293T cells, it was observed that VGLL4 overexpression reduced the level of TEAD1 (e.g., FIG. 5G). To expand on this observation, the effect of VGLL4 on different levels of transfected TEAD1 was studied. VGLL4 consistently reduced the steady-state level of TEAD1 (FIG. 7A). The data indicated that VGLL4 regulates the rate of TEAD1 degradation. The regulation of TEAD1 degradation by VGLL4 was tested by using a photoconvertible reporter to measure the TEAD1 degradation rate. TEAD1 was expressed fused to Dendra2, a fluorescent protein which converts from green to red fluorescence upon transient illumination with 405 nm light (Zhang et al., 2007) (FIGS. 7B-7C). TEAD1-Dendra2 allowed for the degradation of the photoconverted protein to be monitored in real time, independent of ongoing protein synthesis. To allow rapid expression of VGLL4, an inducible expression system was utilized in which addition of doxycycline (DOX) to the culture media rapidly induced VGLL4 expression (FIGS. 8A-8B). In control cells that were not programmed to express VGLL4, DOX treatment did not significantly affect steady-state TEAD1 levels over a 10 hour period (FIG. 7D). In contrast, in cells that expressed VGLL4 upon DOX addition, steady-state TEAD1 levels declined by approximately 50% over the same period (P<0.05; FIGS. 7D-7E). VGLL4 did not reduce the mRNA level of Tead1-Dendra2 (FIG. 8C), indicating that the effect of VGLL4 was post-transcriptional.

DOX-induced VGLL4 expression was combined with live cell imaging of TEAD1-Dendra2 to specifically probe the effect of VGLL4 on TEAD1 stability. Four hours after DOX treatment, TEAD1-Dendra2 was pulse-labeled by photoconversion. In the absence of VGLL4, TEAD1-Dendra2 protein fluorescence intensity dropped by 10% during the first 10 minutes, and then became relatively stable. In contrast, in the presence of VGLL4 the fluorescence intensity of photoconverted TEAD1-Dendra2 declined 18% over the same period (FIG. 7F). This indicated that VGLL4 accelerated TEAD1-Dendra2 degradation. VGLL4 did not affect the stability of Dendra2 itself (FIG. 7F; 5% decline over first 10 min). This indicated that the loss of TEAD1-Dendra2 was specific to the TEAD1 component and that VGLL4 strongly influences TEAD1 stability.

Major pathways for protein degradation can include the proteasome and cysteine, serine, and threonine peptidases. To determine if these candidate pathways are involved in VGLL4-mediated TEAD1 degradation, the effect of MG132 (a universal proteasome inhibitor (Lee et al., 1998)), leupeptin (an inhibitor of serine and threonine peptidases (Umezawa, 1976)) and E64 (an inhibitor specific to cysteine proteases (Barrett et al., 1982)) on VGLL4-mediated TEAD1 degradation was studied. In 293T cells co-transfected with TEAD1 and VGLL4, leupeptin and E64, but not MG132, reduced VGLL4-mediated reduction of TEAD1 steady-state levels (FIG. 8D). Without being bound by theory, this indicated that VGLL4 triggers TEAD1 degradation through cysteine proteases and not the proteasome. Pulse-labeled TEAD1-Dendra2 and live cell imaging was used to confirm that E64 reduced the destabilizing effect of VGLL4 on TEAD1-Dendra2 (FIG. 7F). Together, the data indicated that VGLL4 is sufficient to stimulate TEAD1 degradation through a cysteine protease-dependent pathway.

Previously, VGLL4 was shown to antagonize YAP-TEAD1 transcriptional activity by competitively binding to TEAD, displacing YAP (Koontz et al., 2013). The data indicates an additional mechanism, in which VGLL4 binding to TEAD1 promotes its degradation and thereby reduces the amount available to interact with YAP. To test additional mechanisms, the TEAD1 luciferase reporter 8xGTIIC-Luci (Dupont et al., 2011) was used as a read out of TEAD1-YAP transcriptional activity. 293T cells were co-transfected with 8xGTIIC-Luci, DOX-inducible VGLL4, and YAP expression constructs. One day after transfection, luciferase reporter activity was measured 0-8 hours after Dox treatment. In the control group lacking DOX-inducible VGLL4, reporter activity was relatively stable after addition of DOX (FIG. 7G). In contrast, in cells with DOX-induced VGLL4, luciferase activity declined significantly over this time period (P<0.05, FIG. 7G). To probe the role of protein degradation in the effect of VGLL4, cells were treated with E64, which significantly increased reporter activity 8 hours after DOX treatment (P<0.05; FIG. 7G). This indicated that VGLL4 stimulation of TEAD1 degradation contributed to the decrease in transcriptional activity observed after VGLL4 induction. However, E64 did not completely abrogate the reduction of TEAD1 transcriptional activity caused by VGLL4 induction. In part this reflects incomplete protection by E64 of TEAD1 from VGLL4-mediated degradation (FIG. 8D), as well as the additional inhibitory effect of VGLL4 on YAP recruitment to TEAD1 (FIG. 7H).

In conclusion, VGLL4 was sufficient to stimulate TEAD1 degradation through cysteine-dependent proteases, and VGLL4 antagonized TEAD1-YAP transcriptional activity by stimulating TEAD1 degradation in addition to disrupting TEAD1-YAP interaction.

### Example 5

### Precocious Overexpression of VGLL4[R] in Neonatal Heart Induced Heart Failure

VGLL4 overexpressed in the neonatal heart did not interact with TEAD1 and did not significantly affect neonatal heart growth or function (FIG. 3). However, VGLL4-K225 acetylation in the neonatal heart reduced VGLL4 effect on TEAD1-YAP and thus may have masked VGLL4′s biological activity. Additionally, the data indicated that the VGLL4[R] mutant was refractory to inhibition by K225 acetylation. It was hypothesized that this single amino acid substitution could reveal the cardiac activity of overexpressed VGLL4. To test this effect, a mutant protein was introduced into the neonatal heart by developing and administering AAV9.VGLL4[R]. AAV9.GFP and AAV9.VGLL4 were used as negative controls. TEAD1 interacting proteins were detected by co-immunoprecipitation. Consistent with prior results, significant interaction between TEAD1 and VGLL4 were not detected (FIG. 9A, lane 6 vs. 5). Accordingly, AAV9.VGLL4 did not affect TEAD1 level or TEAD1-YAP interaction. In contrast, VGLL4[R] did interact with TEAD1 (FIG. 9A, see lane 7 vs. 5 and 6). Consistent with this interaction, TEAD1 level was reduced by VGLL4[R] (FIG. 9A, lane 7 vs. 5 and 6), and the balance between TEAD1′s interaction partners was altered, with greater binding to VGLL4[R] and reduced binding to YAP. These results suggest that VGLL4 acetylation at K225 governs its interaction with TEAD1 in the neonatal heart. By blocking K225 acetylation, the K225R mutation revealed the potent effect of VGLL4 on neonatal heart function.

Next, the role of p300 in VGLL4 K225R acetylation in the neonatal heart was addressed. The level of p300 was not affected by overexpression of VGLL4 or VGLL4[R] (FIG. 9B, see lanes 2 and 3 vs. 1). VGLL4 and VGLL4[R] both co-immunoprecipitated with p300 (FIG. 9B, see lanes 6 and 7 vs. 5) in neonatal heart, while this interaction was not detected in adult heart (FIG. 10A). Co-precipitated VGLL4 was acetylated, whereas VGLL4[R] was not detectably acetylated (FIG. 9B, see lane 6 vs. 7). This result validated that the K225R mutation reduced VGLL4 acetylation, and indicated that p300 mediates VGLL4 acetylation in vivo.

Given that VGLL4 K225R mutation increased TEAD1-VGLL4 interaction in neonatal heart at the expense of TEAD1-YAP, the biological effect of this single amino acid substitution was investigated. AAV9.VGLL4[R] was delivered to postnatal day 1 (P1) mice. Littermates treated with AAV9.VGLL4 (wild-type) and AAV9.GFP-treated were used as negative controls. At P8, all three groups had similar heart and body weights (FIG. 10B), and as observed previously, heart function was no different between AAV9.VGLL4 and AAV-GFP groups (FIG. 9C). However, AAV9.VGLL4[R] induced severe myocardial dysfunction and myocardial wall thinning (FIGS. 9C-9D). The AAV9.VGLL4[R]-treated mice failed to grow normally, and 30% died prior to a planned necropsy date at P12 (FIGS. 10B-10C). AAV9.VGLL4[R]-treated mice that survived to P12 had striking ventricular and atrial enlargement that were not observed in either negative control group (FIG. 9E). These mice had lower body weight and higher heart weight than the other two groups (FIG. 9F and FIGS. 10B-10C). Staining of heart sections with picrosirius red showed that AAV9.VGLL4[R] hearts had extensive fibrosis (FIGS. 9G-9H). Interestingly, AAV9.VGLL4 hearts also had mildly but significantly increased fibrosis compared to AAV9.GFP. AAV9. Gene expression measurements by qRTPCR indicated that VGLL4[R] induced cardiac upregulation of Nppa and downregulation of Myh6, changes frequently observed in heart failure (FIGS. 9I-9J).

In conclusion, these results indicated that K225R acetylation regulated VGLL4 activity in the neonatal heart. Blocking K225R uncovered potent VGLL4 activity to disrupt TEAD1-YAP interaction and neonatal heart development and function.

### Example 6

### Activation of VGLL4 in the Neonatal Heart Suppressed Cardiomyocyte Proliferation by Disrupting the YAP-TEAD1 Complex

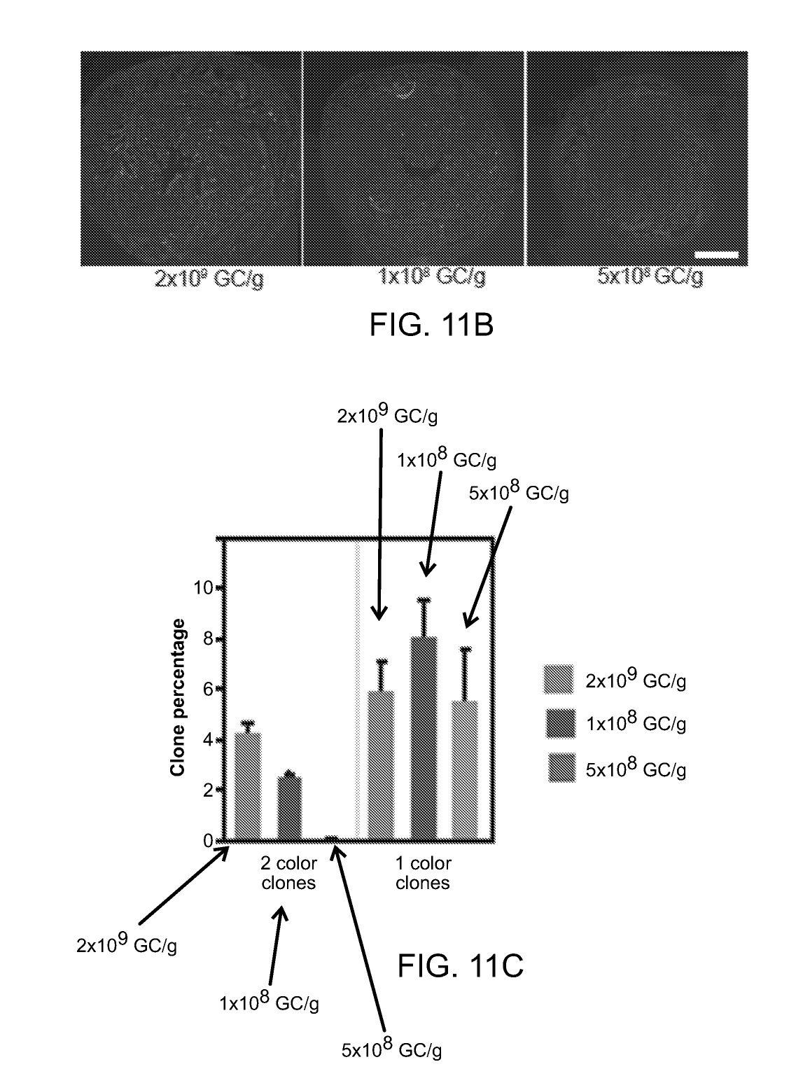

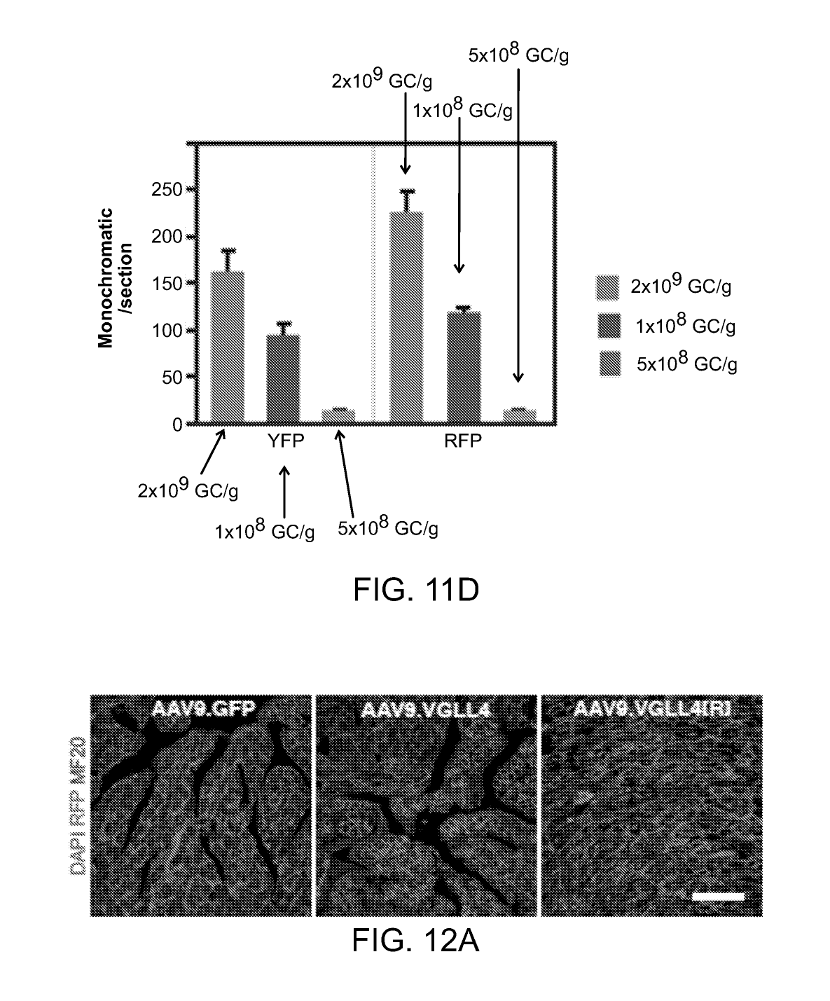

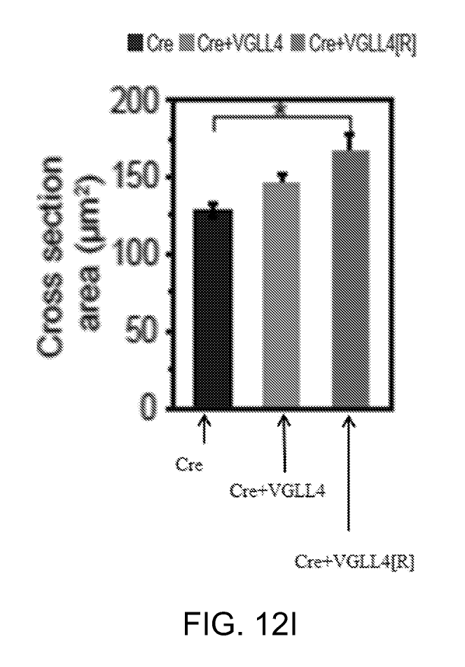

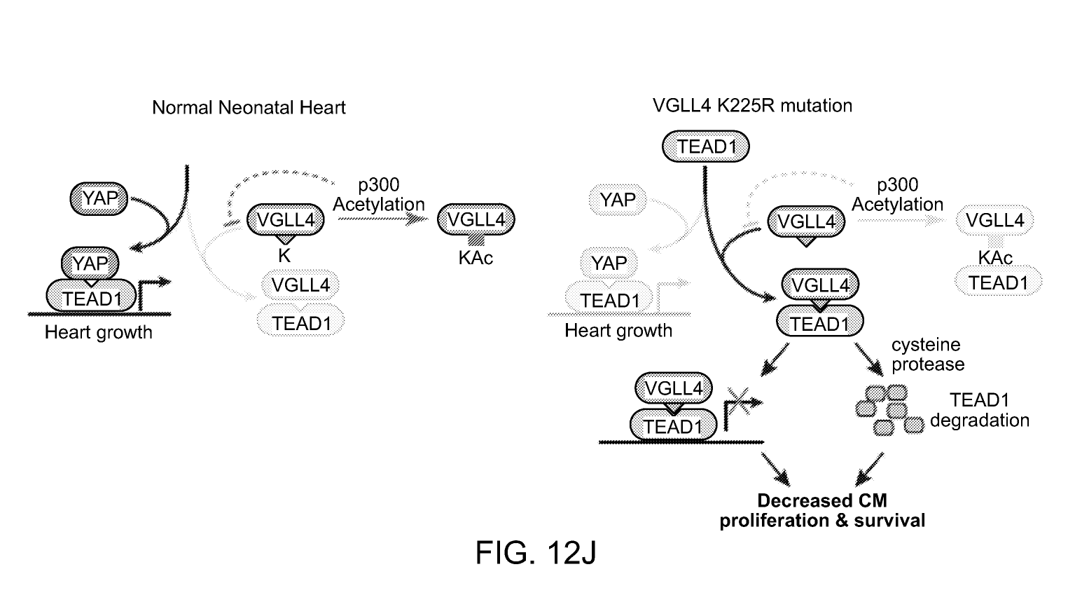

The YAP-TEAD complex promotes cardiomyocyte (CM) proliferation (von Gise et al., 2012; Xin et al., 2011; Heallen et al., 2011; Lin et al., 2014), and loss of this activity caused heart failure at least in part due to reduced CM number (Del Re et al., 2013). In addition, YAP-TEAD has been implicated in regulating CM survival (Del Re et al., 2013). To understand the cellular mechanisms underlying VGLL4[R]-induced heart failure, processes that might alter CM survival (apoptosis or necrosis) or production (proliferation) were investigated. VGLL4[R]did not significantly induce CM apoptosis, as measured by TUNEL staining (FIG. 11A). CMs undergoing necrosis have plasma membranes that are abnormally permeable to macromolecules such as antibodies. Therefore, uptake of injected anti-myosin antibody by CMs has been used as an assay of CM necrosis (Nakayama et al., 2007). To determine if AAV9.VGLL4[R] caused CM necrosis, mouse anti-myosin antibody (MF20) was injected into mouse pups at P7, and the hearts were analyzed at P8. In both negative control groups, MF20+ CMs were rarely observed, whereas MF20+ CMs were readily observed in the AAV9.VGLL4[R] group (FIGS. 12A-12B).

To determine if VGLL4[R] reduced CM proliferation, quantitative pH3 staining was performed. VGLL4[R] strongly decreased the fraction of pH3+ CMs compared to VGLL4 or GFP (FIG. 12C). Because CM multinucleation or polypoidization can dissociate CM M-phase activity from CM number, a clonal analysis was used to more directly probe the effect of VGLL4 on CM proliferation. As described previously (Lin and Pu, 2014), pulse-labeling a low fraction of CMs and later counting the number of CMs in individual labeled clusters can be used to assess the extent of productive CM cell cycle activity. Use of the multi-color Confetti reporter mouse (Snippert et al., 2010), in which Cre stochastically activates expression of one of four fluorescent proteins, further enhances this strategy by allowing one to distinguish chance labeling of two neighboring cells (potentially yielding multichromatic clusters) from expansion of a single CM (monochromatic clusters). AAV9.cTNT::Cre (AAV9.Cre) was injected into the P1 Brainbow pups at a low dose to irreversibly label a small fraction of CMs with one of the four different fluorescent proteins: cyan fluorescent protein (CFP), red fluorescent protein (RFP), nuclear green fluorescent protein (nGFP), and yellow fluorescent protein (YFP). For technical reasons, only RFP and YFP readouts were considered. In pilot experiments, the optimal dose of AAV9.Cre was determined to achieve the desired CM labeling rate (FIGS. 11B-11D). This dose of AAV9.Cre was delivered to P1 Confetti mouse pups. At the same time, AAV9.VGLL4fb or AAV9.VGLL4[R]fb, in which the GFP tag has been replaced by the flag-bio tag, was delivered at doses capable of transducing greater than 90% of cardiomyocytes. In P8 hearts, the frequency of bichromatic and monochromatic cell clusters was determined, where a cluster was defined as two or more labeled, adjacent cells (FIG. 12E). As expected, bichromatic clusters, representing independent labeling of neighboring cells, occurred at similar frequency in AAV9.Cre, AAV9.Cre+ AAV9.VGLL4fb, and AAV9.Cre+ AAV9.VGLL4[R]fb groups (FIG. 12F). In contrast, monochromatic clusters, representing productive CM proliferation, were less common in the VGLL4[R] group (FIG. 12F). Without being bound by theory, this supports the hypothesis that VGLL4[R] overexpression reduces neonatal CM proliferation. Consistent with these measurements of CM proliferation, expression of cell cycle genes Aurka and Cdc20, as well as the canonical TEAD-YAP target gene Ctgf, was reduced in P12 hearts treated with AAV9.VGLL4[R], compared to AAV9.GFP (FIG. 12G).

The reduction of both CM cell cycle activity and CM survival have the potential to result in the reduction of CM number, leading to an increased workload for the remaining CMs. Stressed CMs undergo compensatory hypertrophy, and indeed AAV9.VGLL4[R]-treated CMs were observed to be larger than CMs in the GFP negative control group (FIGS. 12H-12I). In combination with increased cardiac fibrosis, these changes may account for the increased weight of AAV9.VGLL4[R]-treated hearts (FIG. 9F and FIG. 10C).

In conclusion, blocking VGLL4 K225 acetylation revealed the potent effect that VGLL4 has on CM proliferation and survival in the neonatal heart, at least in part through disruption of TEAD1-YAP interaction and destabilization of TEAD1.

The present work identifies VGLL4 as an important negative regulator of cardiac growth driven by YAP-TEAD1, defines VGLL4 acetylation as a novel mechanism to regulate Hippo-YAP signaling and organ growth, and identifies VGLL4 regulation of TEAD1 stability as an additional mechanism whereby VGLL4 modulates YAP-TEAD activity.

### Developmental Regulation of TEAD-Interacting Proteins

The Hippo-YAP pathway controls the growth of mitotic organs, such as liver (Dong et al., 2007), intestine (Camargo et al., 2007), skin (Schlegelmilch et al., 2011), and fetal heart (Heallen et al., 2011; von Gise et al., 2012; Xin et al., 2011), and YAP activation induced pathological hyperplasia and organomegaly. In the adult heart, YAP activation induced limited CM proliferation and was insufficient to cause cardiomegaly (Lin et al., 2014). These observations suggested that unknown mechanisms restrain YAP activity in the adult heart, and the work of Koontz et al. suggested that VGLL4 may be one negative regulatory factor (Koontz et al., 2013). Among the organs that were studied, VGLL4 was most highly expressed in the heart. Cardiac VGLL4 expression was developmentally regulated, with high expression in post-mitotic (adult) CMs and relatively lower expression in mitotic, neonatal CMs. At the same time, YAP and TEAD1 also exhibited developmentally regulated expression, with these factors being expressed more highly in the neonatal than adult heart. Consistent with these changes in protein expression, the main interaction partner of TEAD1 switched from YAP in the neonatal heart to VGLL4 in the adult heart. This developmentally regulated switch in interaction partners is important for normal heart maturation, since precocious formation of TEAD1-VGLL4 complex in the neonatal heart caused cardiac hypoplasia, CM necrosis, and lethal heart failure.

Regulation of YAP-TEAD1 Activity through VGLL4 Acetylation

There are a number of regulatory pathways that converge on YAP to regulate its cellular localization and transcriptional activity. As a transcriptional co-activator, YAP transcriptional activity depends upon its binding to a partner DNA-binding transcription factor. Where examined on a genome-wide scale, TEAD is the major transcription factor partner of YAP (Zanconato et al., 2015; Galli et al., 2015). Functionally, TEAD1 interaction with YAP is essential for fetal heart growth (von Gise et al., 2012). Additionally TEAD1 likely has additional roles in the regulation of muscle gene expression (Yoshida, 2008). Nevertheless, little attention has been directed at mechanisms that control TEAD1 activity and stability.

As reported herein, the VGLL4-TEAD1 interaction is developmentally regulated. Although this is partially explained by developmentally regulated changes in protein expression, the lack of interaction between overexpressed VGLL4 and TEAD1 in the neonatal heart pointed to additional regulatory mechanisms. A second mechanism, VGLL4 acetylation at K225, which normally impedes VGLL4-TEAD interaction in the neonatal heart was discovered (FIG. 6J). Abrogation of acetylation at this residue in the VGLL4[R] mutant precociously impaired VGLL4-TEAD1 interaction, thereby reducing YAP-TEAD1 mitogenic and pro-survival activity (FIG. 6J). Thus, our data show that this post-translational modification is a critical regulatory switch that is essential for neonatal CM proliferation and survival.

One of the major acetyltransferases that acetylates VGLL4 is p300. In the neonatal heart, an interaction between endogenous p300 and VGLL4 was readily detected. This interaction was also developmentally regulated, as it was not detected in adult heart. Decline of p300 levels, alteration of its primary interaction partners, or destabilization of the p300-VGLL4 complex (e.g., due to changes in the composition of each protein's interacting partner complexes) may all contribute to this developmentally reduced interaction. Decreased p300-VGLL4 interaction between neonatal and adult CMs correlated with a decrease in the fraction of VGLL4 that is acetylated. However, VGLL4 acetylation was still present in adult heart, and indeed the absolute level of acetylated VGLL4 was higher. Without wishing to be bound by theory, this suggests that a different acetyltransferase may acetylate VGLL4 in adult CMs. Alternatively, p300 may still acetylate VGLL4, albeit with reduced efficiency that reflects the decline of p300-VGLL4 interaction detectable by co-immunoprecipitation. Understanding the mechanisms that regulate VGLL4 acetylation may lead to therapeutic strategies to enhance VGLL4 acetylation and thereby mitigate its inhibitory effects on YAP-TEAD activity in mature CMs.

Interestingly, VGLL4 appeared to be regulated differently in human and mouse hearts. Whereas mouse VGLL4 increased markedly with postnatal age, its expression in human heart was relatively constant. On the other hand, VGLL4-K225Ac declined with age in human heart. In mouse it increases, but total VGLL4 increases even more, so that the proportion of VGLL4-K225Ac decreases. This suggests that developmental regulation of VGLL4 activity may be achieved in different species by varying combinations of regulated expression or acetylation.

Other major YAP-TEAD regulatory pathways also involve reversible post-translational modifications. For instance, the Hippo kinase cascade phosphorylates YAP to trigger its cytoplasmic sequestration (Huang et al., 2005). This is counter-balanced by YAP dephosphorylation by the protein phosphatase PP2A, a process regulated by YAP interaction with α-catenin (Schlegelmilch et al., 2011). Lysine acetylation is also a reversible post-translational modification that can be removed by deacetylases. Thus in future work it will be interesting to consider whether deacetylases counter-balance VGLL4 acetylation.

### VGLL4 Regulates TEAD1 Stability.

Previous work suggested that VGLL4 suppresses YAP activity by competing for

TEAD binding (Zhang et al., 2014; Koontz et al., 2013). The data provided herein support this mechanism, and uncovered an additional previously unrecognized mechanism by which VGLL4 regulates YAP-TEAD1 activity. It was found that VGLL4 interaction with TEAD1 accelerated TEAD1 protein degradation (FIG. 6J). The cysteine peptidase inhibitors E64 and Leupeptin, but not the proteasome inhibitor MG-132, blocked VGLL4-mediated TEAD1 degradation. However, E64 blockade of this effect of VGLL4 on TEAD1 stability only partially rescued YAP-TEAD1 transcriptional activity, suggesting that VGLL4 regulates YAP-TEAD1 activity by both causing TEAD1 degradation and by inhibiting YAP-TEAD1 interaction. Future work is required to define the mechanism by which VGLL4 enhances TEAD1 degradation, and the specific cysteine peptidases that degrade TEAD1.

VGLL4 Affects Postnatal Cardiac Growth and Maturation by both Suppressing CM Proliferation and Necrosis

These results demonstrated that VGLL4 activity in neonatal heart is blocked by its acetylation at K225. Overriding VGLL4 acetylation by mutating this residue to arginine unmasked the potent effect of VGLL4 gain of function in the neonatal heart. Overexpression of VGLL4[R] in neonatal heart destabilized the TEAD1-YAP complex, decreasing CM proliferation and YAP target gene expression. Moreover, VGLL4[R] induced CM necrosis but not apoptosis. As a result of reduced CM proliferation and increased necrosis, AAV9.VGLL4[R] transduced pups developed heart failure. These data identify an additional, previously unreported role of YAP-TEAD1 in CMs to suppress necrosis. This function of YAP may cross cell types, as YAP loss of function predisposed hepatocytes to undergo necrosis after bile duct ligation (Bai et al., 2012). CM necrosis is an important mechanism of CM loss following experimental myocardial infarction and genetically-induced CM calcium overload (Kajstura et al., 1998; Nakayama et al., 2007). It will be interesting to dissect the mechanisms by which the balance between VGLL4-TEAD and YAP-TEAD governs CM necrosis.

In conclusion, this study identified novel mechanisms that regulate YAP-TEAD activity. Normal deployment of these mechanisms is crucial for neonatal cardiac growth and maturation. Without wishing to be bound by theory, this suggests that manipulation of the balance between VGLL4, TEAD, and YAP activity, and/or suppressing VGLL4-mediated TEAD1 degradation, may be of use for therapeutic cardiac regeneration or repair. Alternatively, augmenting the inhibitory action of VGLL4 or reducing its acetylation may be useful strategies to control oncogenic growth driven by excessive YAP-TEAD activity.

### Example 7

### Transient Expression of YAP

AAV9-YAP has been used to induce YAP pathway activation and improve outcomes in mice after MI (Lin and Pu, 2014). The improvement likely arises from both decreased cardiomyocyte death and increased cardiomyocyte proliferation. However, further clinical development of AAV9-YAP is limited by the potential for oncogenesis in other tissues, such as the liver (Dong et al., 2007).

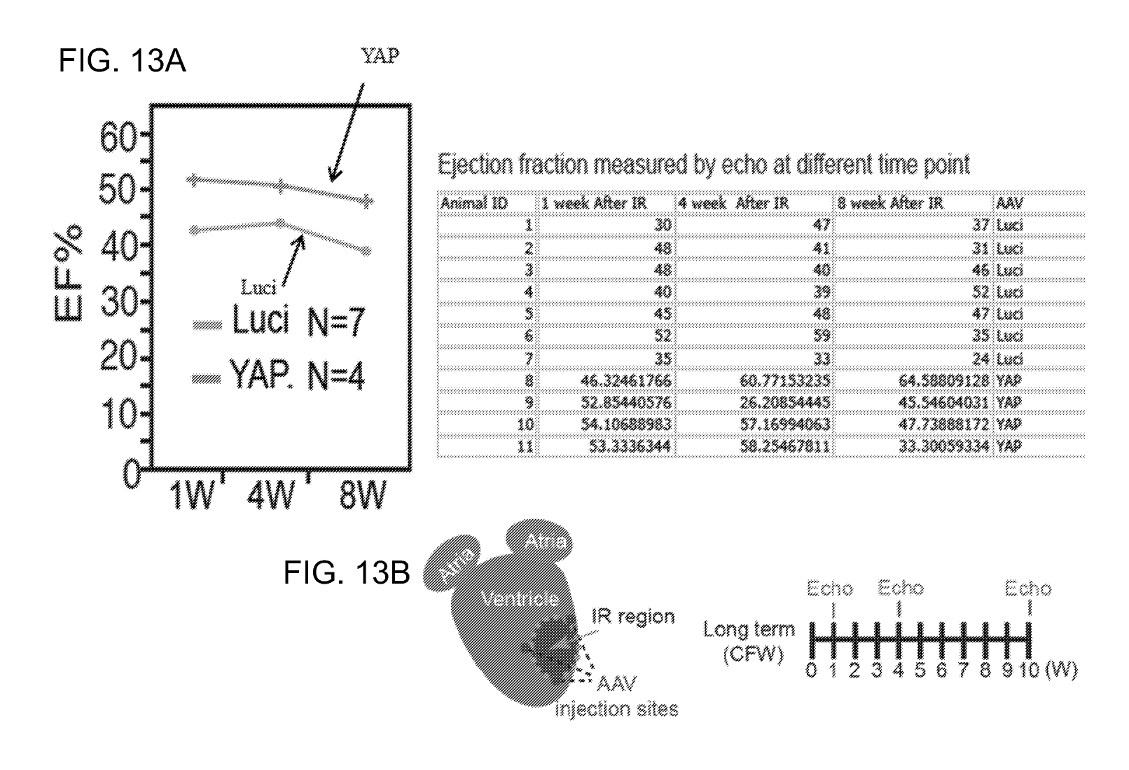

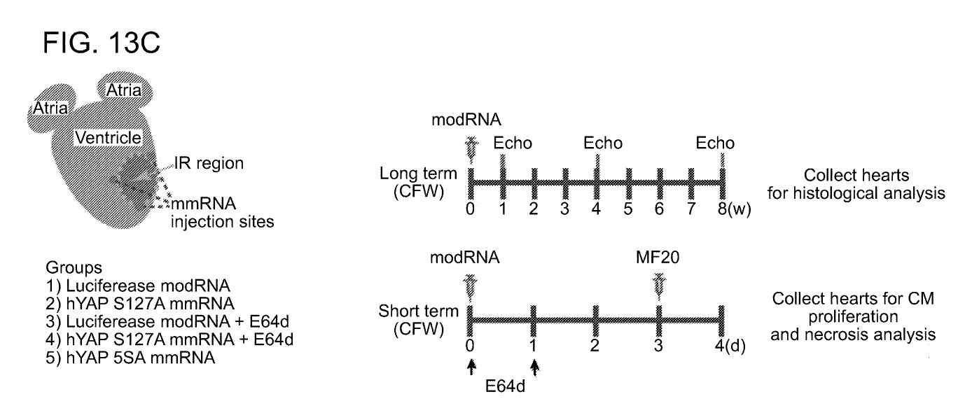

Significantly, the benefit of AAV9-YAP was found to largely occur in the first week after MI (FIGS. 13A and 13B). Ejection fraction (%) was consistently about 10% higher up to 8 weeks after I/R in animals receiving AAV9-YAP compared to those receiving control AAV9-Luciferase. Without intending to be bound by theory, this indicates that transient activation of YAP immediately after MI may yield benefit with minimal risk of cancer.

Administration of YAP modRNA is also able to improve cardiac repair in ischemia/reperfusion (I/R) (FIG. 13C).

YAP transcriptional activity is normally dampened in the adult heart by proteolysis of YAP's transcriptional partner TEAD, and this proteolysis can be blocked by the protease inhibitor E64. A variant, E64d, is currently in clinical testing for non-cardiac indications (Traumatic Brain Injury; Chen et al., 2013) and appears to have a good safety profile.

In the rodents, E64 has different effect on different heart failure model. E64 has been tested in some heart disease models. In Dahl salt-sensitive hypertensive rats, E64 treatment attenuated high salt diet induced heart failure (Cheng et al., 2008). In a mouse myocardial infarction model, E64 did not show a beneficial effect (Dong et al., 2007). Modified mRNA is an efficient means to deliver genes to the heart. Importantly, modified mRNA is transiently expressed. Thus it could be used to express YAP after MI yet avoid its oncogenic potential.

### Example 8

### aYAP modRNA Treatment at the Time of Reperfusion Improved Heart Function and Suppressed Cardiac Hypertrophy following Ischemia/Reperfusion

Five to six week-old male C57/BL6 mice were subject to ischemia/reperfusion (FR) cardiac injury. Ischemia was triggered by left anterior descending coronary artery (LAD) ligation. Five to ten minutes after LAD ligation, 100 μl 0.5 μg/μl modRNA encoding human YAP containing an activating S127A mutation (aYAP) and a 3xFLAG tag was injected into the ischemic myocardium. In control mice, modRNA encoding luciferase (Zangi, L. et al. Nat Biotechnol 31, 898-907 (2013)) was injected instead of aYAP. Red fluorescent beads (Life Technologies #F8834) were injected into the left ventricular (LV) chamber to mark the perfused tissue; myocardium not marked by the red beads was defined as the area at risk. 50 minutes after LAD ligation, the ligature was released to reperfuse the affected myocardium. Sham controls underwent the same procedure, but the ligature was not tied around the LAD, and no modRNA was delivered. All investigators were blinded to treatment group.

To measure the effects of aYAP modRNA, short term and long term studies were performed (FIG. 14A). In the short term study, hearts were collected 2 days after ischemia/reperfusion and modRNA injection. Compared with the luciferase modRNA control “Luci modRNA”), YAP mRNA levels in aYAP modRNA treated mice were 5-fold higher (FIG. 14B). Furthermore, aYAP modRNA was successfully translated into aYAP protein as shown by immunoblotting (FIG. 14C).

At 2 days after ischemia/reperfusion and modRNA treatment, the size of control hearts that received modRNA following ischemia/reperfusion (Luci modRNA+IR) was larger than hearts injected with aYAP modRNA following ischemia/reperfusion (“aYAP modRNA+IR”) (FIG. 15A), whereas the extent of injury (area at risk) was not different between the two groups. Heart weight and tibia length ratio were used as parameters for measuring cardiac hypertrophy (Yin, F. C., et al., Am J Physiol 243, H941-H947 (1982)). In the aYAP modRNA+IR group, the heart weight to tibia length ratio was significantly lower than that of the control Luci modRNA+IR group, and was not significantly different from that of the sham control (FIG. 15B). IR injury initiated an inflammatory response in which macrophages and neutrophils were recruited to the injured region. Reducing inflammation following IR is thought to improve myocardial recovery (Epelman, S., et al., Nat Rev Immunol 15, 117-129 (2015)). In sham control heart sections, only a few mononuclear cells were observed. In control Luci modRNA+IR hearts, mononuclear cells were greatly enriched in the area at risk. The aYAP ModRNA+IR hearts had fewer mononuclear cells than the Luci modRNA+IR control hearts (FIG. 15C). These data indicate that aYAP modRNA reduced cardiac inflammation following ischemic reperfulsion.

In the long term study, mouse heart function was measured at 1 week and 4 weeks after I/R and modRNA treatment. In the sham control that was not subjected to I/R, heart systolic function, as measured by the ejection fraction, was in the normal range 1 week after surgery, and did not change even at 4 weeks after surgery. 1 week after FR and treatment with control Luci modRNA, the majority of the mice had poor cardiac function. Their ejection fractions were at least 20% lower than those observed in sham mice. At 4 weeks after I/R, control Luci+IR mice did not show a significant improvement in EF relative to their values at 1 week, and were significantly reduced compared to sham. In the aYAP modRNA+IR group, the ejection fraction was reduced at 1 week compared to sham and comparable to Luci+IR. However, in the YAP modRNA+IR group, the ejection fraction was improved at 4 weeks compared to 1 week after IR. Paired T-test showed a significant difference between 1 and 4 weeks after I/R in the aYAP modRNA mice but not the Luci modRNA mice (FIG. 16A).

To see if aYAP modRNA reduced long term cardiac hypertrophy remodeling, control hearts and hearts treated with aYAP modRNA were collected 3 months following IR. Though the area at risk was similar, the size of aYAP modRNA+IR hearts was smaller than the size of control hearts in the Luci modRNA+IR group (FIG. 16B) indicating that aYAP modRNA treatment reduced cardiac hypertrophy. Consistent with the short term study results, the heart weight to tibia length ratio was significantly lower in the aYAP modRNA+IR group compared to the control Luci modRNA+IR group, and was not significantly different from that of the sham control (FIG. 16C). These data indicate that aYAP modRNA treatment before reperfusion improved cardiac function and reduced cardiac remodeling following I/R.

Collectively, these data indicate that one dose of aYAP modRNA treatment at the time of reperfusion improved cardiac function after IR injury. Without intending to be bound by theory, it is likely that aYAP modRNA treatment acted by reducing inflammation, cardiomyocyte loss, and cardiac remodeling.

The results described herein were obtained using the following materials and methods.

### Animal Experiments

All animal procedures were approved by the Boston Children's Hospital Animal Care and Use Committee. Rosa26^{BirA} and Rosa26^{mTmG} mice were previously described (Driegen et al., 2005; Muzumdar et al., 2007) and were obtained from Jackson Labs. Tead1^{fb} knock-in mice were generated by targeting the C-terminus of Tead1 in murine embryonic stem cells to introduce FLAG and Bio epitope tags, followed by embryonic stem cell blastocyst injection. After establishing germline transmission, the Frt-neo-Frt resistance cassette was removed using FLP expressing mice. These mice are available through the mutant mouse resource (MMRRC: 037514-JAX). Echocardiography was performed in conscious mice by investigators blinded to genotype or treatment group on a Visual-Sonics Vevo 2100 with Vevostrain software.

### Human Myocardium

Human left ventricular myocardium was obtained from unused donor hearts without known heart disease, under protocols approved by the Institutional Review Boards of the University of Sydney and St. Vincent's Hospital. Myocardial samples from the left ventricle were snap frozen in liquid nitrogen within 2 hours of organ harvest.

### Clonal Analysis of Cardiomyocyte Proliferation

Postnatal day 1 Rosa26^{confetti/+} mouse (Livet et al., 2007) pups were treated with AAV9.Cre together with AAV.Luciferase, AAV9.Vgll4 or AAV9.Vgll4[R], respectively. Seven days after virus injection, hearts were collected and processed for cryosectioning. To quantify the different color clones for each heart, whole heart cross-section images were taken using a Nikon TE2000 epifluorescent microscope and Volocity software (Perkin Elmer). Clone numbers were counted blinded to treatment group.

### Histology and Measurement of Cardiomyocyte Proliferation, Apoptosis, and Necrosis

Hearts were fixed in paraformaldehyde (PFA) 4%, washed in PBS, equilibrated with 30% sucrose, and embedded in OCT. Cryosections (10 μm) were used for H&E staining, picrosirius red-fast green staining and immunostaining. Antibodies used for immunostaining are listed below:

| | | | |
|---|---|---|---|
| Antigen | Company (catalog #) | Origin | Working dilution |

Primary antibodies

| | | | |
|---|---|---|---|
| Cardiac | Abcam (ab56357) | Goat | 1:200 for IF |
| troponin I | | | |
| Flag | Sigma (F3165) | Rabbit | 1:1000 for |
| | | | western blot |
| GAPDH | Sigma(WH0002597M1) | Mouse | 1:200,000 for WB |
| WGA-647 | Life technology | NA | 1:250 for IF |
| YAP | Sigma(Y4770) | Rabbit | 1:1000 for WB |
| HA tag | CST(2367) | Mouse | 1:1000 for WB |
| His tag | Life technology | Mouse | 1:1000 for WB |
| | (R93025) | | |
| GFP | Memorial-Sloan | Mouse | IP 1:100 for |
| | Kettering Monoclonal | | Co-IP |
| Phospho | Upstate (06-570) | Rabbit | 1:200 for IF |
| Histone 3 | | | |
| Tead1 (Tef1) | BD biosciences (610923) | mouse | 1:1000 for WB |
| GFP | Rockland (600-101-215) | Goat | 1:1000 for WB |
| V5 | Life technology | Mouse | 1:1000 for WB |
| | (R960-25) | | |
| p300 | Santa Cruz (sc-585x) | Rabbit | 1:500 for Co-IP |
| | | | and 1:2000 for |
| VGLL4 | Bioss Inc.(bs-9185R) | Rabbit | 1:1000 for WB |
| Myh1e | The Developmental | Mouse | NA |

Secondary antibodies

| | | | |
|---|---|---|---|
| Donkey Anti- | Life | Donkey | 1:500 for IF |
| Goat Alexa488 | technology(A11055) | | |
| Clear blot IP | Thermo fisher (21230) | | 1:400 for WB |
| detection regent | | | |
| Donkey Anti- | Jackson lab | Donkey | 1:10000 for WB |
| Rabbit HRP | (705-035-147) | | |
| Donkey Anti- | Life technology | Donkey | 1:500 for IF |
| Rabbit Alexa555 | (A21206) | | |

CM apoptosis was detected on cryosections using the Roche in situ death detection kit.

To measure CM necrosis, the protocol of Nakayama et al. (Nakayama et al., 2007) was adapted. 1-day-old Rosa26^{mTmG} mouse pups were treated with AAV9. 6 days later, 100 μl MF20 antibody (22 μg/ml) was IP injected into the mouse pups. On day 7 after virus transduction, hearts were collected, fixed, and cryosectioned as described above. To visualize cardiomyocytes which had taken up MF20 antibody in vivo, sections were stained with Alexa 647 conjugated Donkey anti mouse IgG.

Imaging was performed on a Fluoview 1000 confocal microscope, or a Nikon TE2000 epifluores-cent microscope. Quantitation was performed blinded to AAV treatment group by stitching 10x fields across entire heart short axis sections.

### Cardiomyocyte Isolation and Culture

Neonatal rat ventricle cardiomyocytes (NRVMs) were isolated from 2-days-old Wistar Rat (Charles River) using the Neomyts cardiomyocyte dissociation kit (Cellutron). Isolated cardiomyocytes were cultured on fibronectin coated coverslips. For 24 hours after dissociation, NRVMs were cultured in the presence of fetal bovine serum (FBS, 10%), when they were changed to 1% horse serum and cultured for an additional 24 hours. Cells were then fixed with PFA (4%) fixation.

### Duolink In Situ PLA Assay

PFA (4%) fixed NRVMs were permeabilized with PBS containing 0.1% Triton X-100. Duolink PLA assay was carried out using the Sigma Duolink® In Situ Red Starter Kit Mouse/Rabbit (Duo92101-1KT). To visualize cardiomyocytes, cells were co-stained with goat originated cardiac troponin I antibody (Abcam). Briefly, NRVMs were incubated at 4 degree over night with primary anti-bodies against VGLL4, TEAD1 and Cardiac Troponin I. On the second day, PLA assay was carried out following the kit protocol. Alexa 488 conjugated Donkey anti goat secondary antibody was added at the signal amplification step.

### AAV9 Packaging

AAV9.Vgll4 or Vgll4[R] were cloned into ITR-containing AAV plasmid (Penn Vector Core P1967) harboring the chicken cardiac TNT promoter, to obtain pAAV.cTnT::Vgll4-GFP and pAAV.cTnT::Vgll4[R]-GFP, respectively. AAV9 was packaged in 293T cells with AAV9:Rep-Cap and pHelper (pAd deltaF6, Penn Vector Core) and purified and concentrated by gradient centrifugation (Lin et al., 2014). AAV9 titer was determined by quantitative PCR. The standard AAV9 dose used for neonatal mice was 2.5×10¹⁰ GC/g. At this dose, over 90% of CMs are transduced routinely.

### His-TEAD1[211-427] Expression and Purification

Murine TEAD1 residues 211-427, containing the YAP and VGLL4 binding domain, were cloned into the pET28a (Novagen) in frame with polyhistidine tag (His) using BamHI and Notl (NEB). The recombinant plasmid was transformed into E. coli BL21 (DE3) cells. A single colony was then used to inoculate 200 ml of LB with 25 μg/ml kanamycin at 37° C. At OD600˜0.5, expression was induced with 0.67 mM isopropyl-β-D-thiogalactopyranoside (IPTG). The culture was further shaken at 18° C. for 16-20 h. Cells were pelleted, then suspended in 10 ml lysis buffer (50 mM Tris-HCl, pH 7.4, 150 mM NaCl, 1 mM PMSF, 10 mM β-mercaptoethanol, and 10 mM imidazole), followed by the addition of 1 mg/ml lysozyme. After incubating on ice for 30 min, the suspension was sonicated (5 reps, 10 sec on, 30 sec off, amplitude 75) with Branson Digital Sonifier equipped with a microtip and subsequently centrifuged at 20,000 g for 20 min at 4° C. The supernatant was incubated with 2 ml Ni-NTA Agarose (Qiagen) for 3 h at 4° C. The resin was washed with 20 column volumes of wash buffer (50 mM Tris-HCl, pH7.4, 150 mM NaCl, 1 mM PMSF, 10 mM (β-mercaptoethanol and 20 mM imidazole).

The protein was eluted with 4-column volumes of elution buffer (50 mM Tris-HCl, pH7.4, 150 mM NaCl, 1 mM PMSF, 10 mM (β-mercaptoethanol and 300 mM imidazole). His-TEAD1[211-427] was concentrated and further purified by size exclusion chromatography with a Superdex 200 increase 10/300 GL column (GE Health Sciences) pre-equilibrated in a buffer of 50 mM Tris-HCl, pH7.4, 150 mM NaCl, 1 mM PMSF, 10 mM β-mercaptoethanol.

### Synthetic VGLL4 TDU Domain Peptides

Wild type and acetylated peptide containing VGLL4 TDU domain and V5 epitope were synthesized by LifeTein LLC. The synthesized peptides were purified with HPLC to reach 95% purity and their molecular weight analyzed by electrospray ionization (ESI) mass spectrometry. The sequences are depicted in FIG. 6A.

### Co-Immunoprecipitation and In Vitro VGLL4 Acetylation Analysis

Cell or tissue soluble protein extracts for co-immunoprecipitation were prepared in lysis buffer (20 mM Tris HCl (pH 8), 137 mM NaCl, glycerol (10%), Triton X-100 (1%), 2 mM EDTA). Protease inhibitor cocktail (Roche) was added to the lysis buffer immediately before use. The protein solution was diluted with 1 volume of IP buffer (Lysis buffer without glycerol), and then was pre-cleared with protein A Dynabeads (Life Technology, 10008D). Antibody or IgG was added to the pre-cleared ex-tract, and antibody bound protein complexes were pulled down with pre-equilibrated protein A Dynabeads. After three washes, the immunoprecipitated proteins were eluted with lx sodium dodecyl sulfate (SDS) loading buffer.

For analysis of VGLL4 acetylation, 293T cells were cotransfected with VGLL4-GFP and other indicated plasmids and cultured for 48 hours. 2 hours before harvest, cells were treated with 5 μM trichostatin A (TSA; Cayman chemical, CAS 58880-19-6). GFP antibody was used to pull down VGLL4-GFP in the presence of 5 μM TSA. Acetylation was analyzed using panacetylated lysine antibody (Cell Signaling Technology, 9441S) or mass spectrometry (Harvard Medical School Taplin Biological Mass Spectrometry Facility).

### Affinity Measurement by Photonic Crystal Nanobeam Sensor.

Affinity measurement was performed using nanobeam photonic sensors consisting of photonic crystal nanobeam cavities for protein sensing and polymer spot-size converters for efficient on-and-off chip light coupling (Liang et al., 2013; Quan et al., 2010). Polydimethylsiloxane (PDMS) microfluidic channels were integrated on the sensor chip for sample delivery. The photonic crystal nanobeam cavities confine the optical energy into nanoscale dimensions, and build up high quality factor (Q-factor) resonances. The nanobeam cavity consists of a tapered array of holes with periodicity 330 nm along a 600 nm wide ridge waveguide. The radii of the holes were tapered from 240 nm in the center of the cavity to 100 nm to both ends of the cavity, designed by the deterministic method described in (Quan and Loncar, 2011). The device was fabricated as described (Quan and Loncar, 2011). Protein binding was measured by monitoring the resonance shift of the nanobeam cavity.

100 ng/mL His-TEAD1 [211-427] was first flowed to the nanobeam sensor via PDMS microfluidic channels, together with 4 mM sodium cyanoborohydride (Sigma) in PBS. After 2 hour-incubation at room temperature, the nanobeam sensor was washed by PBS flow for 10 min. Different concentrations of VGLL4 or acetylated VGLL4 were consecutively injected into the channel. The tunable laser (Santec) was used to scan the input wavelength and collected the signal transmitted through the cavity. The resonance shift was obtained by fitting the resonance with Lorentz curve.

mVGLL4 K216 Acetylation-Specific Antibody Generation.

Antigen design and antibody generation was carried out by Yenzym Antibodies, LLC. The peptide antigen corresponds to mouse VGLL4 (209-222, EHFRRSLGKNYKEPE), in which K216 was acetylated. Rabbits were given four immunizations and acetylation specific antibodies were isolated by affinity purification.

### TEAD1-Dendra2 Merge Protein Time Lapse Imaging

293T cells were cultured on glass bottom 35mm dishes. One day after plasmid transfection, cells were treated with Dox in the absence or presence of E64. Live cell imaging was performed with a Nikon TE2000 epifluorescent microscope equipped with an on-stage chamber that controlled CO2, humidity, and temperature. Green Dendra2 protein was converted to emit red fluorescence by illuminating for 30 seconds with 405 nm light. Photoconversion was considered complete 3 minutes after the 405 nm light illumination, at which point time lapse imaging commenced. Images were acquired at 1 image/min. For each group, 6 different regions of interest were used for quantifying red fluorescence intensity (RFI).

### Gene Expression

Real time PCR was performed with Syber Green or Taqman detection using BioRad CFX96 Real time system. PCR primers are listed below:

Primers

| Gene* | Forward | Reverse |
|---|---|---|
| mCTGF | CCACCCGAGTTACCAATGA | GACAGGCTTGGCGATTTTAG |
| mCCNA2 | GCCTTCACCATTCATGTGG | TTGCTCCGGGTAAAGAGACAG |
| mCDC20 | TTCGTGTTCGAGAGCGATT | ACCTTGGAACTAGATTTGCCAG |
| mAurka | GGGTGGTCGGTGCATGCTC | GCCTCGAAAGGAGGCATCCCCAC |
| mMyh6 | CTCTGGATTGGTCTCCCAG | GTCATTCTGTCACTCAAACTCTG |
| mGapdh | CAGGTTGTCTCCTGCGACT | GGCCTCTCTTGCTCAGTGTC |
| mTead1 | TACTGCCATCCACAACAAG | TGCTGCACAAAGGGCTTGAC |
| ABI | | |
| Taqman | Gene Assay Number | |
| mNppa | PN4453320 | |
| mGapdh | 4352339E | |

### Statistics

Values are expressed as mean ±SEM. For two group comparisons, the Student's t-test was used to test for statistical significance. To analyze data containing more than two groups, ANOVA with the Tukey HSD post-hoc test was used. Both tests were performed using JMP 10.0 (SAS).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

The following references are cited herein:
Bai, H., Zhang et al. (2012). Yes-associated protein regulates the hepatic response after bile duct ligation. Hepatology 56, 1097-1107.
Barrett, A. J et al. (1982). L-trans-Epoxysuccinyl-leucylamido(4-guanidino)butane (E-64) and its analogues as inhibitors of cysteine proteinases including cathepsins B, H and L. Biochem J 201, 189-198.
de Boer, E. et al. (2003). Efficient biotinylation and single-step purification of tagged transcription factors in mammalian cells and transgenic mice. Proceedings of the National Academy of Sciences 100, 7480-85.
Camargo, F. D. et al. (2007). YAP1 increases organ size and expands undifferentiated progenitor cells. Curr Biol 17, 2054-060.
Chan, H. M. et al. (2001). p300/CBP proteins: HATs for transcriptional bridges and scaffolds. J Cell Sci 114, 2363-373.
Chen, H. et al. (2004). BMP10 is essential for maintaining cardiac growth during murine cardiogenesis. Development 131, 2219-231.
Chen, H. H. et al. (2004). Vgl-4, a novel member of the vestigial-like family of transcription cofactors, regulates alphal-adrenergic activation of gene expression in cardiac myocytes. J. Biol. Chem. 279, 30800-06.
Del Re, D. P et al. (2013). Yes-associated protein isoform 1 (Yap1) promotes cardiomyocyte survival and growth to protect against myocardial ischemic injury. J Biol Chem 288, 3977-988.
Dong, J. et al. (2007). Elucidation of a universal size-control mechanism in Drosophila and mammals. Cell 130, 1120-133.
Driegen, S. et al. (2005). A generic tool for biotinylation of tagged proteins in transgenic mice. Transgenic Res 14, 477-482.
Dupont, S. et al. (2011). Role of YAP/TAZ in mechanotransduction. Nature 474, 179-183.
Epelman, S., Liu, P. P. & Mann, D. L. Role of innate and adaptive immune mechanisms in cardiac injury and repair. Nat Rev Immunol 15, 117-129 (2015).
Galli, G. G. et al. (2015). YAP Drives Growth by Controlling Transcriptional Pause Release from Dynamic Enhancers. Mol Cell 60, 328-337.
He, A. et al. (2012). PRC2 directly methylates GATA4 and represses its transcriptional activity. Genes Dev 26, 37-42.
Heallen, T. et al. (2013). Hippo signaling impedes adult heart regeneration. Development 140, 4683-690.
Heallen, T. et al. (2011). Hippo pathway inhibits Wnt signaling to restrain cardiomyocyte proliferation and heart size. Science 332, 458-461.
Heidenreich, P. A. et al.(2013). Forecasting the impact of heart failure in the United States: a policy statement from the American Heart Association. Circ Heart Fail 6, 606-619.
Huang, J. et al. (2005). The Hippo signaling pathway coordinately regulates cell proliferation and apoptosis by inactivating Yorkie, the Drosophila Homolog of YAP. Cell 122, 421-434.
Jiao, S. et al. (2014). A peptide mimicking VGLL4 function acts as a YAP antagonist therapy against gastric cancer. Cancer Cell 25, 166-180.
Kajstura, J. et al. (1998). Myocyte proliferation in end-stage cardiac failure in humans. Proceedings of the National Academy of Sciences 95, 8801-05.
Koontz, L. M. et al.(2013). The Hippo effector Yorkie controls normal tissue growth by antagonizing scalloped-mediated default repression. Dev Cell 25, 388-401.
Lee, Y. et al. (1998). The cardiac tissue-restricted homeobox protein Csx/Nkx2.5 physically associates with the zinc finger protein GATA4 and cooperatively activates atrial natriuretic factor gene expression. Molecular and cellular biology 18, 3120-29.
Li, J. et al. (2014). Alpha-Catenins Control Cardiomyocyte Proliferation by Regulating Yap Activity. Circ Res
Liang, F. et al. (2013). Scalable photonic crystal chips for high sensitivity protein detection. Optics express 21, 32306-312.
Lin, Z., and Pu, W. T. (2014). Strategies for Cardiac Regeneration and Repair. Sci. Transl. Med. 6, 239rv1.
Lin, Z., and Pu, W. T. (2014). Harnessing Hippo in the heart: Hippo/Yap signaling and applications to heart regeneration and rejuvenation. Stem Cell Res
Lin, Z., and Pu, W. T. (2015). Releasing YAP From an α-Catenin Trap Increases Cardiomyocyte Proliferation. Circ Res 116, 9-11.
Lin, Z. et al.(2014). Cardiac-specific YAP activation improves cardiac function and survival in an experimental murine MI model. Circ Res 115, 354-363.
Lin, Z. et al.(2014). Cardiac-Specific YAP Activation Improves Cardiac Function and Survival in an Experimental Murine Myocardial Infarction Model. Circ Res
Livet, J. et al. (2007). Transgenic strategies for combinatorial expression of fluorescent proteins in the nervous system. Nature 450, 56-62.
Nakayama, H. et al. (2007). Ca2+- and mitochondrial-dependent cardiomyocyte necrosis as a primary mediator of heart failure. J Clin Invest 117, 2431-444.
Muzumdar, M. D. et al. (2007). A global double-fluorescent Cre reporter mouse. Genesis 45, 593-605.
Pan, D. (2010). The hippo signaling pathway in development and cancer. Dev. Cell. 19, 491-505.
Prasad, K.-M. R. et al. (2011). Robust cardiomyocyte-specific gene expression following systemic injection of AAV: in vivo gene delivery follows a Poisson distribution. Gene Therapy 18, 43-52.
Quan, Q. et al. (2011). Deterministic design of wavelength scale, ultra-high Q photonic crystal nanobeam cavities. Optics express 19, 18529-542.
Quan, Q. et al. (2010). Photonic crystal nanobeam cavity strongly coupled to the feeding waveguide. arXiv preprint arXiv:1002.131
Schlegelmilch, K. et al.(2011). Yapl acts downstream of alpha-catenin to control epidermal proliferation. Cell 144, 782-795.
Snippert, H. J. et al. (2010). Intestinal crypt homeostasis results from neutral competition between symmetrically dividing Lgr5 stem cells. Cell 143, 134-144.
Soderberg, 0. et al. (2006). Direct observation of individual endogenous protein complexes in situ by proximity ligation. Nat Methods 3, 995.
Umezawa, H. (1976). Structures and activities of protease inhibitors of microbial origin. Methods Enzymol 45, 678-695.
von Gise, A. et al.(2012). YAP1, the nuclear target of Hippo signaling, stimulates heart growth through cardiomyocyte proliferation but not hypertrophy. Proc. Natl. Acad. Sci U S A 109, 2394-99.
Walsh, S. et al. (2010). Cardiomyocyte cell cycle control and growth estimation in vivo—an analysis based on cardiomyocyte nuclei. Cardiovasc Res 86, 365-373.
Xin, M. et al. (2013). Hippo pathway effector Yap promotes cardiac regeneration. Proc. Natl. Acad. Sci U S A 110, 13839-844.
Xin, M. et al. (2011). Regulation of insulin-like growth factor signaling by Yap governs cardiomyocyte proliferation and embryonic heart size. Sci Signal 4, ra70.
Yang, D. et al. (2014). High sensitivity and high Q-factor nanoslotted parallel quadrabeam photonic crystal cavity for real-time and label-free sensing. Applied Physics Letters 105, 063118.
Yin, F. C., Spurgeon, H. A., Rakusan, K., Weisfeldt, M. L. & Lakatta, E. G. Use of tibial length to quantify cardiac hypertrophy: application in the aging rat. Am J Physiol 243, H941-H947 (1982).
Yoshida, T. (2008). MCAT elements and the TEF-1 family of transcription factors in muscle development and disease. Arterioscler. Thromb. Vasc Biol. 28, 8-17.
Zanconato, F. et al. (2015). Genome-wide association between YAP/TAZ/TEAD and AP-1 at enhancers drives oncogenic growth. Nat. Cell Biol. 17, 1218-227.
Zangi, L., Lui, K. O., von Gise, A., Ma, Q., et al. Modified mRNA directs the fate of heart progenitor cells and induces vascular regeneration after myocardial infarction. Nat Biotechnol31, 898-907 (2013).
Zhang, H. et al. (2014). Yapl is required for endothelial to mesenchymal transition of the atrioventricular cushion. J Biol Chem
Zhang, Z. D. et al. (2007). Statistical analysis of the genomic distribution and correlation of regulatory elements in the ENCODE regions. Genome Res 17, 787-797

1. A method of inducing regeneration and/or reducing cardiomyocyte loss in a cardiac tissue of a subject, the method comprising transiently increasing the level, expression, or activity of a Yap polypeptide in a cell or progenitor thereof in the subject, thereby inducing regeneration and/or reducing cardiomyocyte loss in the cardiac tissue.

2. The method of claim 1, wherein the cell death is by apoptosis or necrosis.

3. A method of increasing cardiac function or reducing cardiac hypertrophy in a subject following ischemic reperfusion injury, the method comprising administering to the subject a YAP polypeptide or polynucleotide encoding said polypeptide, thereby increasing cardiac function or reducing cardiac hypertrophy in the subject.

4. The method of claim 1, wherein the method further comprises administering to the subject an agent that inhibits cathepsin B.

5. A method of treating a myocardial infarction or symptom thereof in a subject, the method comprising transiently administering to the subject an agent that inhibits cathepsin B, wherein the agent is administered prior to, during or following myocardial infarction, thereby treating the myocardial infarction or a symptom thereof.

6. The method of any onc of claim 1, wherein the YAP polypeptide comprises one or more activating mutations.

7. The method of claim 6, wherein the activating mutation is selected from the group consisting of S61A, S109A, S127A, S164A, S381A, S127A mutation, W1, W2, and W1W2.

8. The method of claim 4, wherein the agent is a epoxysuccinyl, vinyl sulfone or nitrile based compound.

9. The method of claim 8, wherein the agent is selected from the group consisting of E64d, E64c, JPM-OEt, CA-030, CA-074, NS134, NS-629, LNC-NS-629, PK1, and ASM7.

10. The method of claim 9, wherein the agent is E64d.

11. The method of claim 3, wherein the polynucleotide is a synthetic, modified RNA molecule.

12. (canceled)

13. The method of claim 12, wherein the at least two modified nucleosides are selected from the group consisting of 5-methylcytidine (5mC), N6-methyladenosine (m6A), 3,2′-O-dimethyluridine (m4U), 2-thiouridine (s2U), 2′ fluorouridine, pseudouridine, N1-methyl-pseudouridine, 2′-O-methyluridine (Um), 2′ deoxy uridine (2′ dU), 4-thiouridine (s4U), 5-methyluridine (m5U), 2′-O-methyladenosine (m6A), N6,2′-O-dimethyl adenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m-2,7G), N2,N2,7-trimethylguanosine (m-2,2,7G), and inosine (I).

14. (canceled)

15. The method of claim 11, wherein the synthetic, modified RNA molecule further comprises a poly(A) tail, a Kozak sequence, a 3′ untranslated region, a 5′ untranslated region, or any combination thereof, and wherein the poly(A) tail, Kozak sequence, 3′ untranslated region, 5′ untranslated region can optionally comprise one or modified nucleosides selected from the group consisting of 5-methylcytidine (5mC), N6-methyladenosine (m6A), 3,2′-O-dimethyluridine (m4U), 2-thiouridine (s2U), 2′ fluorouridine, pseudouridine, N1-methyl-pseudouridine, 2′-O-methyluridine (Um), 2′ deoxy uridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m-2,7G), N2,N2,7-trimethylguanosine (m-2,2,7G), and inosine (I).

16. (canceled)

17. The method of claim 1, wherein the cell is present in a cardiac tissue.

18. The method of claim 11, wherein the modified RNA is administered to the tissue by direct injection, contacting the tissue with an implantable device comprising, or coated with the synthetic, modified RNA molecule, or delivering the synthetic, modified RNA molecule via a catheter or an endoscope.

19. (canceled)

20. The method of claim 11, wherein the concentration of synthetic, modified RNA molecule of greater than 100 ng/μl.

21. The method of claim 11, wherein the concentration of synthetic, modified RNA molecule of between 1-25 μg/μl.

22. (canceled)

23. The method of claim 1, wherein the subject has or is at risk for developing a myocardial infarction, congestive heart failure, cardiomyopathy, myocardial infarction, tissue ischemia, cardiac ischemia, tissue repair, and trauma injury.

24. The method of claim 1, wherein the subject has had or is planning to have cardiac surgery, has an ischemia condition, or is in need of a stent placement.

25. A method for expressing a YAP protein in a cell, the method comprising contacting the cell with a synthetic, modified RNA molecule encoding a YAP polypeptide or a composition comprising a synthetic, modified RNA molecule encoding a YAP polypeptide.

26-27. (canceled)


SEQUENCE LISTING (38 sequences)

SEQ ID NO: 1 - protein, 504 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 2 - DNA, 5234 bp, Homo sapiens
gccgccgccagggaaaagaaagggaggaaggaaggaacaagaaaaggaaataaagagaaaggggaggcggggaaaggcaacgagctgtccggcctccgtcaagggagttggagggaaaaagttctcaggcgccgcaggtccgagtgcctcgcagcccctcccgaggcgcagccgccagaccagtggagccggggcgcagggcgggggcggaggcgccggggcgggggatgcggggccgcggcgcagccccccggccctgagagcgaggacagcgccgcccggcccgcagccgtcgccgcttctccacctcggcccgtggagccggggcgtccgggcgtagccctcgctcgcctgggtcagggggtgcgcgtcgggggaggcagaagccatggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcagccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatagagccctcaggcagactgaattctaaatctgtgaaggatctaaggagacacatgcaccggaaatttccataagccagttgcagttttcaggctaatacagaaaaagatgaacaaacgtccagcaagatactttaatcctctattttgctcttccttgtccattgctgctgttaatgtattgctgacctctttcacagttggctctaaagaatcaaaagaaaaaaactttttatttcttttgctattaaaactactgttcattttgggggctgggggaagtgagcctgtttggatgatggatgccattccttttgcccagttaaatgttcaccaatcattttaactaaatactcagacttagaagtcagatgcttcatgtcacagcatttagtttgttcaacagttgtttcttcagcttcctttgtccagtggaaaaacatgatttactggtctgacaagccaaaaatgttatatctgatattaaatacttaatgctgatttgaagagatagctgaaaccaaggctgaagactgttttactttcagtattttcttttcctcctagtgctatcattagtcacataatgaccttgattttattttaggagcttataaggcatgagacaatttccatataaatatattaattattgccacatactctaatatagattttggtggataattttgtgggtgtgcattttgttctgttttgttgggttttttgttttttttgtttttggcagggtcggtgggggggttggttggttggttggttttgtcggaacctaggcaaatgaccatattagtgaatctgttaatagttgtagcttgggatggttattgtagttgttttggtaaaatcttcatttcctggttttttttaccaccttatttaaatctcgattatctgctctctcttttatatacatacacacacccaaacataacatttataatagtgtggtagtggaatgtatccttttttaggtttccctgctttccagttaatttttaaaatggtagcgctttgtatgcatttagaatacatgactagtagtttatatttcactggtagtttaaatctggttggggcagtctgcagatgtttgaagtagtttagtgttctagaaagagctattactgtggatagtgcctaggggagtgctccacgccctctgggcatacggtagatattatctgatgaattggaaaggagcaaaccagaaatggctttattttctcccttggactaatttttaagtctcgattggaattcagtgagtaggttcataatgtgcatgacagaaataagctttatagtggtttaccttcatttagctttggaagttttctttgccttagttttggaagtaaattctagtttgtagttctcatttgtaatgaacacattaacgactagattaaaatattgccttcaagattgttcttacttacaagacttgctcctacttctatgctgaaaattgaccctggatagaatactataaggttttgagttagctggaaaagtgatcagattaataaatgtatattggtagttgaatttagcaaagaaatagagataatcatgattatacctttatttttacaggaagagatgatgtaactagagtatgtgtctacaggagtaataatggtttccaaagagtattttttaaaggaacaaaacgagcatgaattaactcttcaatataagctatgaagtaatagttggttgtgaattaaagtggcaccagctagcacctctgtgttttaagggtctttcaatgtttctagaataagcccttattttcaagggttcataacaggcataaaatctcttctcctggcaaaagctgctatgaaaagcctcagcttgggaagatagatttttttccccccaattacaaaatctaagtattttggcccttcaatttggaggagggcaaaagttggaagtaagaagttttattttaagtactttcagtgctcaaaaaaatgcaatcactgtgttgtatataatagttcataggttgatcactcataataattgactctaaggcttttattaagaaaacagcagaaagattaaatcttgaattaagtctggggggaaatggccactgcagatggagttttagagtagtaatgaaattctacctagaatgcaaaattgggtatatgaattacatagcatgttgttgggattttttttaatgtgcagaagatcaaagctacttggaaggagtgcctataatttgccagtagccacagattaagattatatcttatatatcagcagattagctttagcttagggggagggtgggaaagtttggggggggggttgtgaagatttagggggaccttgatagagaactttataaacttctttctctttaataaagacttgtcttacaccgtgctgccattaaaggcagctgttctagagtttcagtcacctaagtacacccacaaaacaatatgaatatggagatcttcctttacccctcaactttaatttgcccagttatacctcagtgttgtagcagtactgtgatacctggcacagtgctttgatcttacgatgccctctgtactgacctgaaggagacctaagagtcctttccctttttgagtttgaatcatagccttgatgtggtctcttgttttatgtccttgttcctaatgtaaaagtgcttaactgcttcttggttgtattgggtagcattgggataagattttaactgggtattcttgaattgcttttacaataaaccaattttataatctttaaatttatcaactttttacatttgtgttattttcagtcagggcttcttagatctacttatggttgatggagcacattgatttggagtttcagatcttccaaagcactatttgttgtaataacttttctaaatgtagtgcctttaaaggaaaaatgaacacagggaagtgactttgctacaaataatgttgctgtgttaagtattcatattaaatacatgccttctatatggaacatggcagaaagactgaaaaataacagtaattaattgtgtaattcagaattcataccaatcagtgttgaaactcaaacattgcaaaagtgggtggcaatattcagtgcttaacacttttctagcgttggtacatctgagaaatgagtgctcaggtggattttatcctcgcaagcatgttgttataagaattgtgggtgtgcctatcataacaattgttttctgtatcttgaaaaagtattctccacattttaaatgttttatattagagaattctttaatgcacacttgtcaaatatatatatatagtaccaatgttacctttttattttttgttttagatgtaagagcatgctcatatgttaggtacttacataaattgttacattattttttcttatgtaatacctttttgtttgtttatgtggttcaaatatattctttccttaaactcttaaaaaaaaaa

SEQ ID NO: 3 - RNA, 13 nt, Unknown
  Description of Unknown Kozak sequence
gccgccrccaugg

SEQ ID NO: 4 - DNA, 500 bp, Artificial Sequence
  Description of Artificial Sequence Synthetic polynucleotide
  misc_feature (1)..(500): This sequence may encompass 1-500 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - DNA, 150 bp, Artificial Sequence
  Description of Artificial Sequence Synthetic polynucleotide
  misc_feature (1)..(150): This sequence may encompass 1-150 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - DNA, 120 bp, Artificial Sequence
  Description of Artificial Sequence Synthetic polynucleotide
  misc_feature (1)..(120): This sequence may encompass 90-120 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - protein, 15 aa, Mus sp.
EHFRRSLGKNYKEPE

SEQ ID NO: 8 - DNA, 19 bp, Artificial Sequence
  Description of Artificial Sequence Synthetic primer
ccacccgagttaccaatga

SEQ ID NO: 9 - DNA, 19 bp, Artificial Sequence
  Description of Artificial Sequence Synthetic primer
gccttcaccattcatgtgg

SEQ ID NO: 10 - DNA, 19 bp, Artificial Sequence
  Description of Artificial Sequence Synthetic primer
ttcgtgttcgagagcgatt

SEQ ID NO: 11 - DNA, 19 bp, Artificial Sequence
  Description of Artificial Sequence Synthetic primer
gggtggtcggtgcatgctc

SEQ ID NO: 12 - DNA, 19 bp, Artificial Sequence
  Description of Artificial Sequence Synthetic primer
ctctggattggtctcccag

SEQ ID NO: 13 - DNA, 19 bp, Artificial Sequence
  Description of Artificial Sequence Synthetic primer
caggttgtctcctgcgact

SEQ ID NO: 14 - DNA, 19 bp, Artificial Sequence
  Description of Artificial Sequence Synthetic primer
tactgccatccacaacaag

SEQ ID NO: 15 - DNA, 20 bp, Artificial Sequence
  Description of Artificial Sequence Synthetic primer
gacaggcttggcgattttag

SEQ ID NO: 16 - DNA, 21 bp, Artificial Sequence
  Description of Artificial Sequence Synthetic primer
ttgctccgggtaaagagacag

SEQ ID NO: 17 - DNA, 22 bp, Artificial Sequence
  Description of Artificial Sequence Synthetic primer
accttggaactagatttgccag

SEQ ID NO: 18 - DNA, 23 bp, Artificial Sequence
  Description of Artificial Sequence Synthetic primer
gcctcgaaaggaggcatccccac

SEQ ID NO: 19 - DNA, 23 bp, Artificial Sequence
  Description of Artificial Sequence Synthetic primer
gtcattctgtcactcaaactctg

SEQ ID NO: 20 - DNA, 20 bp, Artificial Sequence
  Description of Artificial Sequence Synthetic primer
ggcctctcttgctcagtgtc

SEQ ID NO: 21 - DNA, 20 bp, Artificial Sequence
  Description of Artificial Sequence Synthetic primer
tgctgcacaaagggcttgac

SEQ ID NO: 22 - protein, 16 aa, Caenorhabditis elegans
QEEVAAHFRRSLSGKW

SEQ ID NO: 23 - protein, 16 aa, Caenorhabditis elegans
TLSVADHFRRALLGKG

SEQ ID NO: 24 - protein, 16 aa, Drosophila sp.
ASQVDEHFSRALNYNN

SEQ ID NO: 25 - protein, 16 aa, Drosophila sp.
EASVDDHFAKALGETW

SEQ ID NO: 26 - protein, 16 aa, Drosophila sp.
MCDIDEHFRRSLGENY

SEQ ID NO: 27 - protein, 16 aa, Homo sapiens
GSVVDEHFSRALGQAI

SEQ ID NO: 28 - protein, 16 aa, Homo sapiens
TGSVDDHFAKALGDTW

SEQ ID NO: 29 - protein, 16 aa, Homo sapiens
SSVVDEHFSRALSNIK

SEQ ID NO: 30 - protein, 14 aa, Drosophila sp.
DIDEHFRRSLGENY

SEQ ID NO: 31 - protein, 16 aa, Danio rerio
DPVIEEHFRRSLGKNY

SEQ ID NO: 32 - protein, 16 aa, Xenopus sp.
DPVVEEHFRRSLGKNY

SEQ ID NO: 33 - protein, 16 aa, Gallus gallus
DPVVEEHFRRSLGKNY

SEQ ID NO: 34 - protein, 16 aa, Mus sp.
DPVVEEHFRRSLGKNY

SEQ ID NO: 35 - protein, 16 aa, Bos taurus
DPVVEEHFRRSLGKNY

SEQ ID NO: 36 - protein, 16 aa, Homo sapiens
DPVVEEHFRRSLGKNY

SEQ ID NO: 37 - protein, 68 aa, Artificial Sequence
  Description of Artificial Sequence Synthetic polypeptide
DPVVEEHFRRSLGKNYKEPEPAPNSVSITGSVDDHFAKALGDTWLQIKAAKDGAGKPIPNPLLGLDST

SEQ ID NO: 38 - protein, 68 aa, Artificial Sequence; identical to SEQ ID NO: 37
  Description of Artificial Sequence Synthetic polypeptide
  MOD_RES (14)..(14): Ac-Lys